(19) United States
(12) Patent Application Publication
(10) Pub. No.: US 2026/0014008 A1
(43) Pub. Date: Jan. 15, 2026

(54) DRUG COATED BALLOON FOR MAIN AND SIDE BRANCH VESSELS

(71) Applicant: ADVANCED BIFURCATION SYSTEMS INC., Livermore, CA (US)

(72) Inventors: Ashur Bourang, Turlock, CA (US); Henry Bourang, Turlock, CA (US)

(21) Appl. No.: 19/267,209

(22) Filed: Jul. 11, 2025

Related U.S. Application Data
(60) Provisional application No. 63/669,799, filed on Jul. 11, 2024.

(51) Int. Cl. A61F 2/954; A61F 2/82; A61F 2/852; A61F 2/958; A61L 29/16; A61M 25/01; A61M 25/09; A61M 25/10

(52) U.S. Cl. CPC A61F 2/954; A61F 2/852; A61F 2/958; A61L 29/16; A61M 25/0108; A61M 25/10; A61F 2002/826; A61F 2250/0067; A61M 2025/0175; A61M 2025/0183; A61M 25/09; A61M 2025/1045; A61M 2025/105

(57) ABSTRACT

A system and method for treating a bifurcated vessel may include a mother catheter with a mother expandable member coupled to a distal portion of a mother elongate shaft. A daughter catheter has a daughter expandable member coupled to a distal portion of a daughter elongate shaft. A therapeutic agent is carried on the daughter expandable member. Radial expansion of the daughter expandable member engages the daughter expandable member with a wall of the daughter vessel and the therapeutic agent elutes from the daughter expandable member into the wall.

### CLAIM OF PRIORITY

The present application is a non-provisional of and claims the benefit of priority to U.S. Provisional Patent Application Ser. No. 63/669,799 (Attorney Docket No. 5133.033PRV) filed on Jul. 11, 2024; the entire contents of which are incorporated herein by reference.

### BACKGROUND

In some examples, the present inventive subject matter relates to medical devices, and more particularly to treatment of a bifurcation with a therapeutic agent.

In medical applications, a stent is an implantable scaffold that is typically delivered percutaneously and deployed in a vein, artery, or other tubular body organ for treating an occlusion, stenosis, aneurysm, collapse, dissection, or weakened, diseased, or abnormally dilated vessel or vessel wall. The stent is radially expanded in situ, thereby expanding and/or supporting the vessel wall or body organ wall. In particular, stents are quite commonly implanted in the coronary, cardiac, pulmonary, neurovascular, peripheral vascular, renal, gastrointestinal and reproductive systems, and have been successfully implanted in the urinary tract, the bile duct, the esophagus, the trachea-bronchial tree and the brain, to reinforce these body organs.

Stents are often used for improving angioplasty results by preventing elastic recoil and remodeling of the vessel wall and for treating dissections in blood vessel walls caused by balloon angioplasty of coronary arteries, as well as peripheral arteries, by pressing together the intimal flaps in the lumen at the site of the dissection. Conventional stents have been used for treating more complex vascular problems, such as lesions at or near bifurcation points in the vascular system, where a secondary artery branches out of a typically larger, main artery, with limited success rates.

Conventional stent technology is relatively well developed. Conventional stent designs typically feature a straight tubular, single-type cellular structure, configuration, or pattern that is repetitive through translation along the longitudinal axis. In many stent designs, the repeating structure, configuration, or pattern has strut and connecting balloon catheter portions that can impede blood flow at vessel bifurcations.

Furthermore, the configuration of struts and connecting balloon catheter portions may obstruct the use of post-operative devices to treat a daughter vessel in the region of a vessel bifurcation. For example, deployment of a first stent in the mother lumen may prevent a physician from inserting a daughter stent through the ostium of a daughter vessel of a vessel bifurcation in cases where treatment of the mother vessel is suboptimal because of displaced diseased tissue (for example, due to plaque shifting or “snow plowing”), occlusion, vessel spasm, dissection with or without intimal flaps, thrombosis, embolism, and/or other vascular diseases. A regular stent is designed in view of conflicting considerations of coverage versus access. For example, to promote coverage, the cell structure size of the stent may be minimized for optimally supporting a vessel wall, thereby preventing or reducing tissue prolapse. To promote access, the cell size may be maximized for providing accessibility of blood flow and of a potentially future implanted daughter stent to daughter vessels, thereby preventing “stent jailing,” and minimizing the amount of implanted material. Regular stent design has typically compromised one consideration for the other in an attempt to address both. Problems the present inventors observed involving daughter jailing, fear of plaque shifting, total occlusion, and difficulty of the procedure are continuing to drive the present inventors into the development of novel delivery systems, which are easier, safer, and more reliable to use for treating the above-indicated variety of vascular disorders. Although conventional stents are routinely used in clinical procedures, clinical data shows that these stents are not capable of completely preventing in-stent restenosis (ISR) or restenosis caused by intimal hyperplasia. In-stent restenosis is the reoccurrence of the narrowing or blockage of an artery in the area covered by the stent following stent implantation. Patients treated with coronary stents can suffer from in-stent restenosis.

Many pharmacological attempts have been made to reduce the amount of restenosis caused by intimal hyperplasia. Many of these attempts have dealt with the systemic delivery of drugs via oral or intravascular introduction. However, success with the systemic approach has been limited.

Systemic delivery of drugs is inherently limited since it is difficult to achieve constant drug delivery to the afflicted region and since systemically administered drugs often cycle through concentration peaks and valleys, resulting in time periods of toxicity and ineffectiveness. Therefore, to be effective, anti-restenosis drugs should be delivered in a localized manner. One approach for localized drug delivery utilizes stents as delivery vehicles. For example, stents seeded with transfected endothelial cells expressing bacterial betagalactosidase or human tissue-type plasminogen activator were utilized as therapeutic protein delivery vehicles. See, e.g., Dichek, D. A. et al., “Seeding of Intravascular Stents With Genetically Engineered Endothelial Cells,” Circulation, 80:1347-1353 (1989). U.S. Pat. No. 5,679,400, International Patent Publication No. WO 91/12779, entitled “Intraluminal Drug Eluting Prosthesis,” and International Patent Publication No. WO 90/13332, entitled “Stent With Sustained Drug Delivery,” which disclose stent devices capable of delivering antiplatelet agents, anticoagulant agents, antimigratory agents, antimetabolic agents, and other anti-restenosis drugs. U.S. Pat. Nos. 6,273,913; 6,383,215; 6,258,121; 6,231,600; 5,837,008; 5,824,048; 5,679,400; and 5,609,629 teach stents coated with various pharmaceutical agents such as Rapamycin, 17-beta-estradiol, Taxol and Dexamethasone. These and all other referenced patents are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

Therefore, given the challenges of current stent technology, a need exists for improved stent delivery systems and methods, particularly for treating bifurcated vessels. At least some of these objectives will be met by the present inventive subject matter which may include the use of a therapeutic agent.

### BRIEF DESCRIPTION OF THE DRAWINGS

FIGS. 1A-1B illustrate an example of a system having an over-the-wire mother catheter and a rapid exchange daughter catheter.

FIGS. 2A-2B illustrate an example of a system having an over-the-wire daughter catheter and a rapid exchange mother catheter.

FIGS. 3A-3B illustrate an example of a system having a rapid exchange mother catheter and a rapid exchange daughter catheter.

FIGS. 4A-4B illustrate an example of a system having an over-the-wire mother catheter and an over-the-wire daughter catheter.

FIGS. 5A-5B illustrate another example of a system having a capture tube, an over-the-wire mother catheter, and a rapid exchange daughter catheter.

FIGS. 6A-6B illustrate another example of a system having a capture tube, an over-the-wire daughter catheter, and a rapid exchange mother catheter.

FIGS. 7A-7B illustrate another example of a system having a capture tube, a rapid exchange mother catheter, and a rapid exchange daughter catheter.

FIGS. 8A-8B illustrate another example of a system having a capture tube, an over-the-wire mother catheter, and an over-the-wire daughter catheter.

FIGS. 9A-9B illustrate yet another example of a system having a removable capture tube, an over-the-wire mother catheter, and a rapid exchange daughter catheter.

FIGS. 10A-10B illustrate yet another example of a system having a removable capture tube, an over-the-wire daughter catheter, and a rapid exchange mother catheter.

FIGS. 11A-11B illustrate yet another example of a system having a removable capture tube, a rapid exchange mother catheter, and a rapid exchange daughter catheter.

FIGS. 12A-12B illustrate yet another example of a system having a removable capture tube, an over-the-wire mother catheter, and an over-the-wire daughter catheter.

FIGS. 13A-13C illustrate still another example of a system having a snap fitting, an over-the-wire mother catheter, and a rapid exchange daughter catheter.

FIGS. 14A-14C illustrate still another example of a system having a snap fitting, an over-the-wire daughter catheter, and a rapid exchange mother catheter.

FIGS. 15A-15C illustrate still another example of a system having a snap fitting, a rapid exchange mother catheter, and a rapid exchange daughter catheter.

FIGS. 16A-16C illustrate still another example of a system having a snap fitting, an over-the-wire mother catheter, and an over-the-wire daughter catheter.

FIGS. 17A-17C illustrate another example of a system having a snap fitting, an over-the-wire mother catheter, and a rapid exchange daughter catheter.

FIGS. 18A-18C illustrate another example of a system having a snap fitting, an over-the-wire daughter catheter, and a rapid exchange mother catheter.

FIGS. 19A-19C illustrate another example of a system having a snap fitting, a rapid exchange mother catheter, and a rapid exchange daughter catheter.

FIGS. 20A-20C illustrate another example of a system having a snap fitting, an over-the-wire mother catheter, and an over-the-wire daughter catheter.

FIGS. 21A-21B illustrate yet another example of a system having an over-the-wire mother catheter and a rapid exchange daughter catheter.

FIGS. 22A-22B illustrate yet another example of a system having an over-the-wire daughter catheter and a rapid exchange mother catheter.

FIGS. 23A-23B illustrate yet another example of a system having a rapid exchange mother catheter and a rapid exchange daughter catheter.

FIGS. 24A-24B illustrate yet another example of a system having an over-the-wire mother catheter and an over-the-wire daughter catheter.

FIGS. 25A-25B, 26A-26B, 27A-27B, 28A-28B, 29A-29B, and 30A-30B illustrate an example of a method for treating a bifurcation.

FIG. 31 illustrates an example of a stent.

FIG. 32 illustrates an example of a system having a mother catheter and a daughter catheter.

FIG. 33 highlights the distal portion of the system illustrated in FIG. 32.

FIG. 34 illustrates alignment of the stents in FIGS. 32-33.

FIG. 35 illustrates a cross-section of a stent crimped over a mother catheter and a daughter catheter.

FIG. 36 illustrates a stent disposed over a mother catheter and a daughter catheter.

FIG. 37 illustrates a stent disposed over a mother catheter and a daughter catheter, and a stent disposed over the daughter catheter.

FIGS. 38A-38M illustrate an example of a method for treating a bifurcation.

FIGS. 39A-39M illustrate another example of a method for treating a bifurcation.

FIGS. 40A-40H illustrate various stents that may be used with the systems and methods disclosed herein to treat bifurcations.

FIGS. 41A-41B illustrate examples of balloon configurations.

FIGS. 42A-42C illustrate engagement of a side branch stent with a main branch stent.

FIGS. 43A-43B illustrate other configurations of a side branch stent engaging a main branch stent.

FIGS. 44-46 illustrate still other configurations of engagement of a side branch stent with a main branch stent.

FIGS. 47A-47D illustrate interdigitation of a side branch stent and a main branch stent.

FIG. 48 illustrates another example of a balloon catheter.

FIGS. 49A-49D include schematic views illustrating aspects of twist resolution techniques of dual catheter systems described herein.

FIG. 50 is a pictorial view of an example pocket.

FIG. 51 illustrates example stents installed using example twist resolution techniques of dual catheter systems described herein.

FIG. 52 illustrates regions of an expandable member.

FIGS. 53A-53G illustrate examples of different regions of a balloon carrying a therapeutic agent.

FIGS. 54A-54K illustrate an example of treating a bifurcated vessel with an expandable member carrying a therapeutic agent.

FIGS. 55A-55J illustrate examples of different configurations of therapeutic agents and polymers.

FIGS. 56A-56D illustrate examples of coating the therapeutic agents on balloons and/or stents.

FIGS. 57A-57F illustrate examples of balloons and stents.

### DETAILED DESCRIPTION

The present inventive subject matter relates to delivery systems for delivery of stents and/or a therapeutic agent to vessel bifurcations having a main branch and a side branch, and is generally configured to at least partially deliver a therapeutic agent to a portion of the side branch. However, this is not intended to be limiting, and one of skill in the art will appreciate that the devices and methods described herein may be used for treating other regions of the bifurcation or the body.

The scientific community is slowly moving away from a main branch vs. side branch model and nomenclature. It is now well accepted that a “mother” vessel bifurcates into two “daughter vessels,” the two vessels that are anatomically after the carina. The vessel that appears to be the continuation of the mother vessel is usually less angulated. The other vessel is frequently smaller in diameter and may be commonly referred to as the side branch, or a daughter vessel. Therefore, in this specification, the terms “main branch,” “trunk,” or “mother vessel” may be used interchangeably. Also in this specification, the terms “side branch vessel” and “daughter vessel” may also be used interchangeably. The terms “main branch stent,” “trunk stent,” or “mother stent” are interchangeable, and the term “side branch stent” is also interchangeable with the term “daughter stent.” In the case where a main branch vessel bifurcates into two equally sized branches, one of the branches may still be considered to be the main branch or mother vessel, and the other branch may be considered a side branch or daughter vessel. In some circumstances, the main branch may bifurcate into two branches where one branch is substantially co-linear with the main branch, while the other branch may be angulated relative to the main branch. In this situation, the co-linear branch distal of the bifurcation may still be referred to as the main branch or mother vessel, and the angulated branch may be referred to as the side branch or daughter vessel.

A variety of catheter designs may be employed to deploy and position the mother and daughter stents. Such catheters may be used in connection with multiple guidewires that terminate in the mother and daughter vessels. These guidewires may be used to facilitate introduction of the catheter, any angioplasty balloons, any stents, and/or to properly orient the stent or balloon within the vessel.

In general, the methods disclosed herein may utilize a catheter system comprising a catheter body having a mother vessel guidewire lumen and a daughter vessel balloon that is independently operable and coupled to the catheter body. The daughter balloon catheter portion has a daughter vessel guidewire lumen. The catheter system further includes a mother catheter balloon, and a stent is disposed over the balloon. The daughter catheter portion extends into the proximal opening of the mother stent and exits the mother stent through a side passage of the mother stent.

According to one method, a mother vessel guidewire is inserted into the mother vessel until a distal end of the mother vessel guidewire passes beyond the ostium of the daughter vessel, and a daughter vessel guidewire is inserted into the mother vessel until a distal end of the daughter vessel guidewire passes into the daughter vessel. To prevent the crossing of guidewires, the two vessels are wired through a guidewire catheter with two lumens to keep the guidewires separate and untangled.

The guidewire catheter is then removed and a wire separator is placed on the wires to keep the guidewires unwrapped. The catheter system is then advanced over the mother and daughter vessel guidewires, with the mother and daughter vessel catheters passing over the mother vessel guidewire and the daughter vessel guidewire. The catheter system is advanced on both wires with the daughter vessel balloon catheter portion distal to the mother balloon catheter portion, leading the system. As the catheter system advances over the wires, the daughter vessel balloon will enter the daughter vessel and may be positioned after or simultaneously with placement of the mother vessel balloon. The mother balloon catheter portion of the catheter system is then advanced distally as far as it can be advanced where it is stopped by the carina. It cannot be advanced beyond the bifurcation site because the tension of the daughter catheter on the mother stent will prevent the mother catheter from moving distally. At this time, the distal portion of the mother stent is beyond the carina in the mother vessel and cannot be advanced any further. This method facilitates advancement of the catheter system to the bifurcation, which may be necessary for tortuous or calcified coronaries. Once the catheter system is in place, the daughter vessel balloon catheter portion is then pulled back relative to the mother catheter so that the proximal part of the daughter balloon is partially within the mother stent. Alignment can be performed with radiopaque markers, in that the proximal markers on the two balloons are next to each other. The operator can then gently push the catheter system distal to maximize apposition to the carina. The daughter balloon, which is now partially under the mother stent, is then inflated to ensure proper alignment of the mother stent. The daughter balloon may also have a stent on its distal portion, which would result in the proximal portion of the mother stent and the daughter stent to expand simultaneously. The daughter balloon is then deflated.

The mother balloon is then inflated, which deploys the mother stent. Kissing, or reinflation, of the two balloons is performed if necessary or for shifting plaque. The catheter system may be removed while the wires remain in place. In this example, or any of the other examples disclosed herein, an angioplasty catheter may be used to predilate the vessel and lesion prior to stenting. In some examples, primary stenting is employed where the stent is deployed without the predilation. The two vessels may be angioplastied separately if predilation is indicated on occasion.

In an alternative method, the mother catheter can be mounted on the daughter vessel guidewire and the daughter catheter can be mounted on the mother vessel guidewire. In daughter vessels with a high degree of angularity, for example, when the bifurcation angle is greater than about 60-70°, the friction between catheters is lower when the operator needs to draw the daughter stent proximally along the main branch and into the mother stent, as opposed to the prior configuration where the daughter stent is drawn along the side branch into the mother stent. The catheter system is advanced so the daughter balloon catheter leads the system and passes the ostium of the daughter vessel, while remaining in the mother vessel. As the catheter system is advanced further, the mother balloon catheter will enter the daughter vessel. The catheter system can only be advanced a certain distance toward the bifurcation, until it is stopped by the carina. It cannot be advanced beyond the bifurcation site because the tension of the daughter catheter on the mother stent will prevent the mother catheter from moving distally. At this time the distal portion of the mother stent is beyond the ostium of the daughter vessel and cannot be advanced any further. While the mother catheter is held in place, the daughter catheter is drawn back such that the proximal portion of the daughter balloon is partially in the mother stent. Alignment can be performed with radiopaque markers, in that the proximal markers on the two balloons are next to each other. The operator can then gently push the catheter system distally to maximize apposition to the carina. A stent on the daughter balloon (which is now partially under the mother stent) is aligned so that when the daughter balloon is inflated, the daughter stent and the proximal portion of the mother stent expand simultaneously and give complete coverage of the mother vessel. The daughter vessel balloon is then deflated. The mother vessel balloon is then inflated, and the distal portion of the mother stent is expanded. A kissing procedure can also be performed if required.

The mother vessel can be stented if necessary, with any commercially available stent. A balloon on a wire could be used as an alternative to the daughter catheter. In an alternative example, the catheter system can be arranged with the daughter balloon portion proximal to the mother balloon portion and advanced over the guidewires to the bifurcation. In the case of the mother catheter on the mother guidewire, the alignment of the mother stent with the ostium of the daughter vessel occurs because tension between the daughter guidewire and mother stent on the mother catheter prevents further advancement of the mother catheter. In the alternative case of the mother catheter on the daughter guidewire, the alignment of the mother stent with the ostium of the mother vessel occurs because tension between the mother guidewire and mother stent on the mother catheter (on the daughter guidewire) prevents further advancement of the mother catheter. In both cases the daughter stent is advanced into alignment with the mother stent and expanded. In some examples, the mother catheter is an over-the-wire (OTW) design and the daughter catheter is a rapid-exchange (RX) design with daughter catheter portion optionally distal thereto. The daughter balloon is placed just distal to the tip of the mother catheter; this arrangement minimizes the overall profile of the catheter system and allows maximal tracking of the arteries. The system may additionally have stents crimped over the balloons. The daughter stent may be any length, but in some examples is approximately half the length of the daughter balloon or mother stent. The proximal end of the mother stent may be crimped only slightly to allow the daughter catheter balloon portion to operate independently so that it may be pushed or pulled without dislodging the mother stent.

An example of a method comprises the following steps:

1. Advance the catheter system to bifurcation, daughter balloon catheter portion and mother balloon catheter portion in their respective vessels.

2. The mother catheter is no longer able to advance because of the tension between the mother stent and daughter catheter.

3. The daughter balloon proximal portion is drawn back into the mother stent and aligned with radiopaque markers.

4. While holding both the mother and daughter catheters tightly, the operator pushes forward lightly.

5. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent. Expansion of the proximal portion of the mother stent and the daughter stent may occur simultaneously.

6. Once the daughter stent is fully deployed, then the mother balloon can be fully expanded to deploy the distal portion of the mother stent.

7. A conventional kissing procedure may be utilized to ensure full apposition. In one particular aspect, the daughter balloon catheter portion may be used without a stent. This allows perfect alignment of the mother stent around the ostium of the daughter vessel. The daughter balloon would be used for the alignment as outlined in step three above, and expands the proximal portion of the mother stent.

In an alternative example, the mother catheter is an over-the-wire (OTW) design and the daughter catheter is a rapid-exchange (RX) design with daughter catheter portion distal thereto. The system may additionally have stents crimped over the balloons. The daughter stent may be less than the length of the mother balloon or stent, although this is not intended to be limiting, and the daughter stent may be any length. The proximal end of the mother stent may be partially crimped to allow the daughter catheter balloon portion to operate independently, so that it may be pushed or pulled without restriction and minimum friction, and without dislodging or affecting the mother stent. An example of a method comprises the following steps:

1. Looping the OTW so that one operator can hold both guide wires with one hand and then push both catheters with the other.

2. Advance the catheter system to the bifurcation, daughter balloon catheter portion and mother balloon catheter portion aligned in their respective vessels, as disclosed in steps two through three in the above example.

3. While holding both the mother and daughter catheters tightly, push the catheter system forward until the mother balloon catheter portion is stopped at the carina.

4. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent.

5. Once the daughter stent is fully deployed, then the mother balloon can be fully expanded to deploy the distal portion of the mother stent.

6. A conventional kissing procedure may be utilized to ensure full apposition.

In one particular aspect, the daughter balloon catheter portion may be used without a stent. This would allow perfect alignment of the mother stent around the ostium of the daughter vessel. The daughter balloon would be used for the alignment as outlined in step three above, and expand the proximal portion of the mother stent.

In an alternative example, the mother catheter is an over-the-wire design and the daughter catheter is a rapid-exchange design with daughter catheter portion distal thereto. The system may additionally have stents crimped over the balloons. The daughter stent may be approximately half the length of the mother balloon or stent, but this is not intended to be limiting, and the daughter stent may be any length. The proximal end of the mother stent may be partially crimped to allow the daughter catheter balloon portion to operate independently, so that it may be pushed or pulled without dislodging the mother stent. An example method comprises the following steps:

1. Place the daughter catheter over the guidewire in the daughter vessel and slide the system into the guide catheter without placing the mother balloon over a guidewire at this time. After the leading daughter catheter enters the coronary artery and just before the mother catheter exits the guide catheter, insert the mother guidewire through the mother catheter and into the mother vessel, then push the system out of the guide catheter over the two guidewires. This method mitigates wire wrap.

2. Advance the catheter system to the bifurcation, daughter balloon catheter portion and mother balloon catheter portion aligned in their respective vessels.

3. Advance the catheter system to bifurcation, daughter balloon catheter portion and mother balloon catheter portion aligned in their respective vessels, as disclosed in step two in the above example. Pull the daughter catheter back until the proximal markers on both balloons are aligned.

4. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent.

5. Once the daughter stent is fully deployed, then the mother balloon can be fully expanded to deploy the distal portion of the mother stent.

6. A conventional kissing procedure may be utilized to ensure full apposition. In one particular aspect, the daughter balloon catheter portion may be used without a stent. This would allow perfect alignment of mother stent around the ostium of the daughter vessel. The daughter balloon would be used for the alignment as outlined in step three above, and expand the proximal portion of the mother stent.

In an alternative example the mother and daughter systems' balloons are aligned. This example may include the mother stent and daughter stent or either stent. When there is both a mother stent and a daughter stent, the daughter stent is may be shorter than the mother stent, although it may be any length, and in some examples is approximately half the length of the mother stent so that the daughter stent could be mounted on the distal half of the daughter balloon. Furthermore, the proximal portion of the daughter catheter shaft is positioned under the non-uniformly crimped mother stent. The dual stent arrangement reduces the profile compared to a full length stent that covers the entire length of the daughter balloon.

The methods described herein could alternatively include the step of flushing the catheters and the guidewire port to assist with maneuverability. The methods described herein could alternatively include the step of a couple of snap-on couplers that lock the two catheters together. In another particular aspect, each balloon catheter portion may include at least one radiopaque marker. With such a configuration, separation of the markers may be conveniently observed using fluoroscopy to indicate that the balloon catheter portions have passed beyond the ostium and the daughter balloon catheter portion has passed into the daughter vessel, thus aligning the passage of the stent with the ostium of the daughter vessel. In another particular aspect, the catheter systems design is contemplated to cover combinations of rapid exchange and over the wire; for visualization purposes the hybrid versions may be used because they are easier to distinguish while using fluoroscopy.

In another particular aspect, the proximal balloon may be differentially expandable, such that one end of the balloon may expand prior to the other end. In another particular aspect, the proximal balloon catheter portion may receive a stent that can be crimped under variable pressure to allow the distal balloon catheter portion freedom of movement.

In another particular aspect, a stent may be crimped over the proximal balloon catheter portion and the stent may be designed to deploy with variable profile to better oppose the patient anatomy.

In another particular aspect, the distal balloon catheter portion may be delivered via a pull-away or peel-away capture tube. All of the above examples may utilize mother vessel stents having any diameter, with diameter optionally ranging from about 2.5 to about 5 millimeters, and daughter vessel stent having any diameter, optionally ranging from about 2 to about 5 millimeters. The length of the stents may be any length, optionally in the range of about 4 to about 40 millimeters. The position of a stent on a catheter need not be fixed and may be positioned on either or both catheters.

### Catheter Configurations:

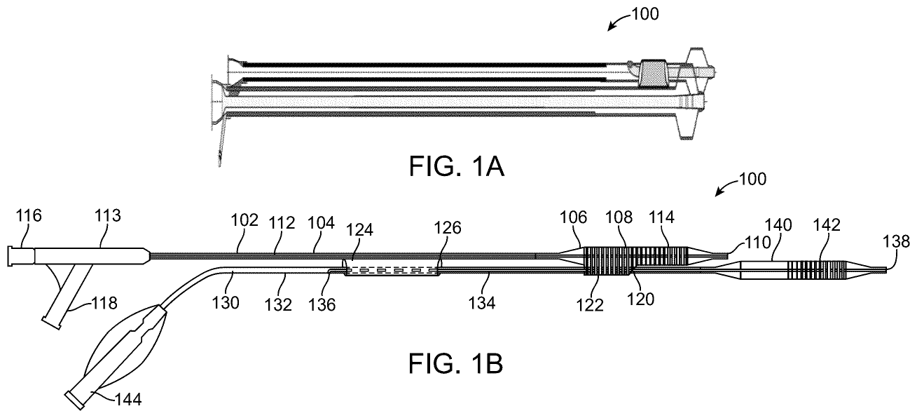

FIG. 1A illustrates an example of the catheter system 100 with a distal daughter balloon catheter portion comprising a balloon with a daughter stent crimped thereon. The daughter stent may be shorter than the mother stent, and it may not be centered on its corresponding balloon in this as well as any other examples disclosed herein. Thus, in some examples, a proximal portion of the daughter balloon remains uncovered by a stent, as will be discussed in greater detail below. In a particular example the daughter stent is optionally about half the length of the mother stent. The distal daughter stent is crimped under standard conditions known in the art. The proximal mother balloon catheter portion comprises a mother balloon and a mother stent. The mother stent is crimped differentially along the longitudinal direction and also differentially circumferentially. In this example, the distal half of the mother stent is crimped under typical conditions to ensure that the mother stent is not dislodged during the alignment with the distal daughter balloon. Further, the proximal portion of the mother stent is crimped under non-standard, relatively loose, conditions to allow the distal daughter balloon catheter portion freedom of movement even though a portion of the daughter balloon catheter portion is circumferentially enclosed. The mother and daughter catheters are slidably attached to each other via a hollow exchange port. The exchange port is embedded in the side of the mother over the wire catheter and has an inner diameter just large enough to allow the insertion of the rapid exchange daughter catheter and balloon. The exchange port may be any length that extends between a proximal portion of the balloons and a distal portion of the catheter connectors, and in this example is about 10 centimeters long, but in some examples varies from about 1 centimeter to about 30 centimeters, and in other examples is about 5 cm to about 10 cm long. The entry for the daughter catheter on the exchange port is proximal and the exit for the daughter catheter is on the distal end of the exchange port. The daughter catheter is loaded through the exchange port and the daughter balloon extends distally from the exit of the exchange port, for example, about 5 centimeters. However, it is possible to have the exchange port any distance from the mother balloon, but in some examples about 1 to about 30 centimeters proximal to the mother balloon. The daughter stent can be crimped on to the balloon after it has been loaded through the exchange port. The exchange port in some examples has a tight fit to reduce catheter profile and may have low friction to allow the operator to easily slide the catheters relative to each other.

FIG. 1B more clearly illustrates the features of the catheter system 100 in FIG. 1A. The catheter system 100 includes a first catheter 102 and a second catheter 130. The first catheter 102 includes an elongate shaft 104 with a radially expandable balloon 106 disposed near a distal end of the elongate shaft 104. A stent 108 having a proximal portion 122, a distal portion 114 and a side hole 120 is disposed over the balloon 106. The distal portion 114 is crimped to the balloon 106 to prevent ejection during delivery, while the proximal portion 122 is partially crimped to the balloon 106 so the second catheter 130 may be slidably advanced or retracted under the proximal portion 122 of stent 108. The first catheter 102 is an over-the-wire (OTW) catheter having a guidewire lumen 112 extending from the distal guidewire port 110 at the distal end of the elongate shaft 104 to the proximal end of the elongate shaft 104 into Y-adapter 113 having a connector 116. The connector 116 is optionally a Luer connector, and this allows easy coupling with a syringe or other device for lumen flushing or injecting contrast media. When unconnected, the guidewire lumen 112 exits via connector 116. A second connector 118, also optionally a Luer connector, allows attachment of an Indeflator or other device to the catheter 102 for inflation of the balloon 106 via an inflation lumen (not shown) in the elongate shaft 104. The first catheter 102 also includes a hollow exchange port tube 124 coupled to the elongate shaft 104. The hollow exchange port tube 124 may be coextruded with the elongate shaft 104, or it may be bonded or otherwise attached thereto using techniques known to those skilled in the art. The hollow exchange port 124 may alternatively be coupled with the elongate shaft 132 of the second catheter 130. The hollow exchange port tube 124 includes a central channel 126 extending therethrough and is sized to slidably receive a portion of the second catheter 130. Radiopaque markers may be placed at different locations along the shaft 104, often near the balloon 106 and/or stent 108, to help mark the proximal and distal ends of the stent or balloon, as well as to facilitate alignment of the two catheters during stent deployment, as discussed elsewhere in this specification.

The second catheter 130 includes an elongate shaft 132 with a radially expandable balloon 140 disposed near a distal end of the elongate shaft 132. A stent 142 is disposed over balloon 140. The stent 142 may have a length that matches the working length of the balloon 140, or the stent length may be shorter than the balloon working length. In some examples, the stent 142 is shorter than the working length of the balloon 140 so that a proximal portion of the balloon 140 is unconstrained by the stent 142, and this unconstrained portion of the balloon 140 may be slidably advanced or retracted through side hole 120 and under proximal portion 122 of stent 108 as will be discussed below. Stent 142 is crimped to balloon 140 to prevent ejection during delivery. At least a portion of balloon 140 and stent 142 are distally offset relative to balloon 106 and stent 108 so as to minimize profile of the device. In this example, the distal stent 142 may be deployed in a main branch of the vessel and the other stent 108 may be deployed in a side branch of the vessel. Alternatively, the distal stent 142 may be deployed in a side branch of a vessel and the other stent 108 may be deployed in the main branch of a vessel.

The second catheter 130 is a rapid exchange catheter (RX) having a guidewire lumen 134 extending from the distal guidewire port 138 at the distal end of the elongate shaft 132 to a proximal guidewire port 136, which is closer to the distal guidewire port 138 than the proximal end of the catheter shaft 132. The proximal guidewire port 136 is also unobstructed by the hollow exchange port tube 124 and optionally proximal thereto. A connector 144, optionally a Luer connector, is connected to the proximal end of the elongate shaft 132 and allows an Indeflator or other device to be coupled with an inflation lumen (not shown) in elongate shaft 132 for inflation of balloon 140. A portion of shaft 132 is disposed in the central channel 126 of the hollow exchange port tube 124, and this helps keep the two catheter shafts 104, 132 parallel and prevents tangling during delivery and as shaft 132 is slidably advanced or retracted relative to shaft 104. Also, another portion of shaft 132 is disposed under proximal portion 122 of stent 108. The second catheter 130 may also be slidably advanced or retracted under the proximal portion 122 of stent 108 so that the shaft 132 passes through the side hole 120 in stent 108. Radiopaque markers may be placed at different locations on the shaft 132, often near the balloon 140 or stent 142, to help mark the proximal and distal ends of the stent 142 or balloon 140, as well as to facilitate alignment of the two catheters during stent deployment, as discussed elsewhere in this specification.

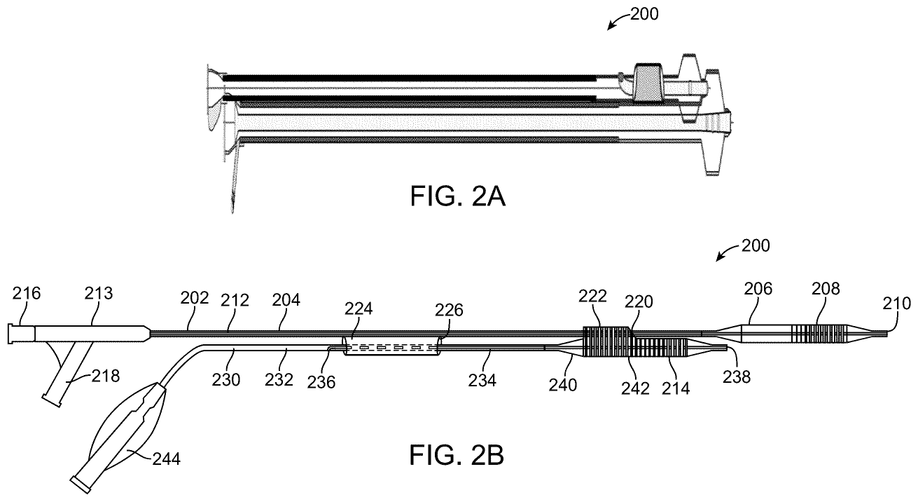

FIG. 2A illustrates a cross-sectional view of one example of a catheter system 200 with the daughter catheter balloon portion distal to the mother balloon portion utilizing the same exchange port as described in FIG. 1A. The mother balloon is may be at least about 5 centimeters distal from the exit of the exchange port. As disclosed above, the mother balloon could be distal from the exchange port from about 1 cm to about 30 centimeters.

FIG. 2B more clearly illustrates the features of the catheter system 200 in FIG. 2A. The catheter system 200 includes a first catheter 202 and a second catheter 230. The first catheter 202 includes an elongate shaft 204 with a radially expandable balloon 206 disposed near a distal end of the elongate shaft 204, and a stent 208 disposed over the balloon 206. The stent 208 may be the same length as the working length of the balloon 206, or it may be shorter. In some examples, the stent 208 is shorter than the working length of balloon 206 such that a proximal portion of balloon 206 remains unconstrained by stent 208. The proximal portion of balloon 206 may be slidably advanced and retracted under stent 242 via side hole 220. Stent 208 is crimped to the balloon 206 to prevent ejection during delivery. The first catheter 202 is an over-the-wire (OTW) catheter having a guidewire lumen 212 extending from the distal guidewire port 210 at the distal end of the elongate shaft 204 to the proximal end of the elongate shaft 204 into Y-adapter 213 having a connector 216. The connector 216 is optionally a Luer connector, and this allows easy coupling with a syringe or other device for lumen flushing or injecting contrast media. When unconnected, the guidewire lumen 212 exits via connector 216. A second connector 218, also optionally a Luer connector, allows attachment of an Indeflator or other device to the first catheter 202 for inflation of the balloon 206 via an inflation lumen (not shown) in the elongate shaft 204. The first catheter 202 also includes a hollow exchange port tube 224 coupled to the elongate shaft 204. The hollow exchange port tube 224 may be coextruded with the first shaft 204, or it may be bonded or otherwise attached thereto using techniques known to those skilled in the art. The hollow exchange port tube 224 may alternatively be coupled with the other shaft 232. The hollow exchange port tube 224 includes a central channel 226 extending therethrough and is sized to slidably receive a portion of the second catheter 230. Radiopaque markers may be placed at different locations along the shaft 204, often near the balloon 206 and/or stent 208, to help mark the proximal and distal ends of the stent 208 or balloon 206, as well as to facilitate alignment of the two catheters during stent deployment, as discussed elsewhere in this specification.

The second catheter 230 includes an elongate shaft 232 with a radially expandable balloon 240 disposed near a distal end of the elongate shaft 232. A stent 242 having a proximal portion 222, a distal portion 214, and a side hole 220 is disposed over balloon 240. The distal portion 214 is crimped to balloon 240 to prevent ejection during delivery, while the proximal portion 222 is partially crimped to balloon 240 so elongate shaft 204 may be slidably advanced or retracted under the proximal portion 222 of stent 242. The stent 242 may optionally have a length that matches the working length of the balloon 240, or the stent length may be shorter than the balloon working length. At least a portion of balloon 206 and stent 208 are distally offset relative to balloon 240 and stent 242 so as to minimize profile of the device. In this example the distal stent 208 may be deployed in a main branch of the vessel and the other stent 242 may be deployed in a side branch of the vessel. Alternatively, the distal stent 208 may be deployed in a side branch of a vessel and the other stent 242 may be deployed in the main branch of a vessel.

The second catheter 230 is a rapid exchange catheter (RX) having a guidewire lumen 234 extending from the distal guidewire port 238 at the distal end of the elongate shaft 232 to a proximal guidewire port 236, which is closer to the distal guidewire port 238 than the proximal end of the catheter shaft 232. The proximal guidewire port 236 is also unobstructed by the hollow exchange port tube 224 and optionally proximal thereto. A connector 244, optionally a Luer connector, is connected to the proximal end of the elongate shaft 232 and allows an Indeflator or other device to be coupled with an inflation lumen (not shown) in elongate shaft 232 for inflation of balloon 240. A portion of shaft 232 is disposed in the central channel 226 of the hollow exchange port tube 224 and this helps keep the two catheter shafts 204, 232 parallel and prevents tangling during delivery and as shaft 232 is slidably advanced or retracted relative to shaft 204. Also, a portion of shaft 204 is disposed under proximal portion 222 of stent 242. The first catheter 202 may be slidably advanced or retracted under the proximal portion 222 of stent 242 so that the shaft 204 passes through the side hole 220 in stent 242. Radiopaque markers may be placed at different locations on the shaft 232, often near the balloon 240 or stent 242, to help mark the proximal and distal ends of the stent 242 or balloon 240, as well as to facilitate alignment of the two catheters during stent deployment, as discussed elsewhere in this specification.

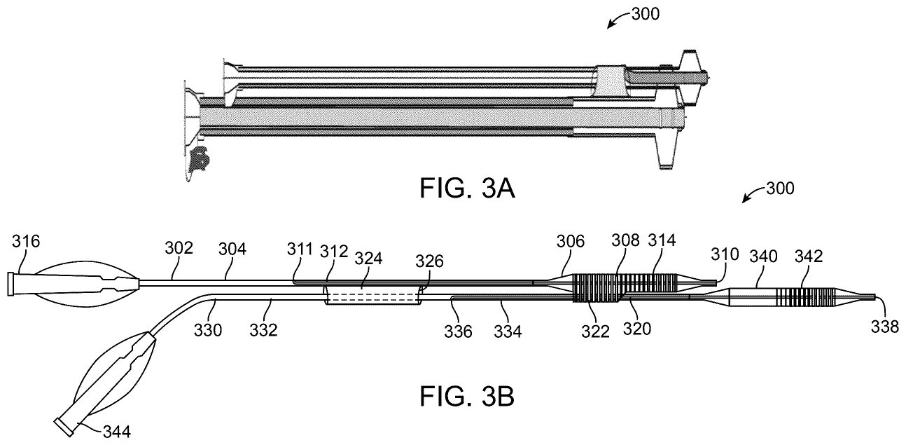

FIG. 3A illustrates a cross-sectional view of one example of a catheter system 300 with the mother and daughter catheters both having a rapid exchange design. In this particular example one of the catheters has a hollow exchange port embedded in its side and the other catheter is loaded through the exchange port. Typically, the catheter is loaded prior to having a stent crimped over the balloon portion.

FIG. 3B more clearly illustrates the features of the catheter system 300 in FIG. 3A. The catheter system 300 includes a first catheter 302 and a second catheter 330. The first catheter 302 includes an elongate shaft 304 with a radially expandable balloon 306 disposed near a distal end of the elongate shaft 304. A stent 308 having a proximal portion 322, a distal portion 314 and a side hole 320 is disposed over the balloon 306. The distal portion 314 is crimped to the balloon 306 to prevent ejection during delivery, while the proximal portion 322 is partially crimped to the balloon 306 so the second catheter 330 may be slidably advanced under the proximal portion 322 of stent 308. The first catheter 302 is a rapid exchange catheter (RX) having a guidewire lumen 312 extending from the distal guidewire port 310 at the distal end of the elongate shaft 304 to a proximal guidewire port 311, which is closer to the distal guidewire port 310 than the proximal end of the catheter shaft 304. A connector 316 is coupled with the proximal end of the elongate shaft 304. The connector 316 is optionally a Luer connector, and this allows easy coupling with an Indeflator or other device for inflation of the balloon 306. The first catheter 302 also includes a hollow exchange port tube 324 coupled to the elongate shaft 304. The hollow exchange port tube 324 may be coextruded with the first shaft 304, or it may be bonded or otherwise attached thereto using techniques known to those skilled in the art. The hollow exchange port tube 324 may alternatively be coupled with the other shaft 332. The hollow exchange port tube 324 includes a central channel 326 extending therethrough and is sized to slidably receive a portion of the second catheter 330. Radiopaque markers may be placed at different locations along the shaft 304, often near the balloon 306 and/or stent 308, to help mark the proximal and distal ends of the stent 308 or balloon 306, as well as to facilitate alignment of the two catheters during stent deployment, as discussed elsewhere in this specification.

The second catheter 330 includes an elongate shaft 332 with a radially expandable balloon 340 disposed near a distal end of the elongate shaft 332. A stent 342 is disposed over balloon 340. The stent 342 may have a length that matches the working length of the balloon 340, or the stent length may be shorter than the balloon working length. In some examples, the stent 342 is shorter than the working length of the balloon 340 so that a proximal portion of the balloon 340 is unconstrained by the stent 342, and this unconstrained portion of the balloon 340 may be slidably advanced or retracted through side hole 320 and under proximal portion 322 of stent 308 as will be discussed below. Stent 342 is crimped to balloon 340 to prevent ejection during delivery. At least a portion of balloon 340 and stent 342 are distally offset relative to balloon 306 and stent 308 so as to minimize profile of the device. In this example the distal stent 342 may be deployed in a main branch of the vessel and the other stent 308 may be deployed in a side branch of the vessel. Alternatively, the distal stent 342 may be deployed in a side branch of a vessel and the other stent 308 may be deployed in the main branch of a vessel.

The second catheter 330 is a rapid exchange catheter (RX) having a guidewire lumen 334 extending from the distal guidewire port 338 at the distal end of the elongate shaft 332 to a proximal guidewire port 336, which is closer to the distal port 338 than the proximal end of the catheter shaft 332. The proximal guidewire port 336 is also unobstructed by the hollow exchange port tube 324 and may be distal thereto. A connector 344, optionally a Luer connector, is connected to the proximal end of the elongate shaft 332 and allows an Indeflator or other device to be coupled with an inflation lumen (not shown) in elongate shaft 332 for inflation of balloon 340. A portion of shaft 332 is disposed in the central channel 326 of the hollow exchange port tube 324, and this helps keep the two catheter shafts 304, 332 parallel and prevents tangling during delivery and as shaft 332 is slidably advanced or retracted relative to shaft 304. Also, another portion of shaft 332 is disposed under proximal portion 322 of stent 308. The second catheter 330 may also be slidably advanced or retracted under the proximal portion 322 of stent 308 so that the shaft 332 passes through the side hole 320 in stent 308. Radiopaque markers may be placed at different locations on the shaft 332, often near the balloon 340 or stent 342, to help mark the proximal and distal ends of the stent 342 or balloon 340, as well as to facilitate alignment of the two catheters during stent deployment, as discussed elsewhere in this specification.

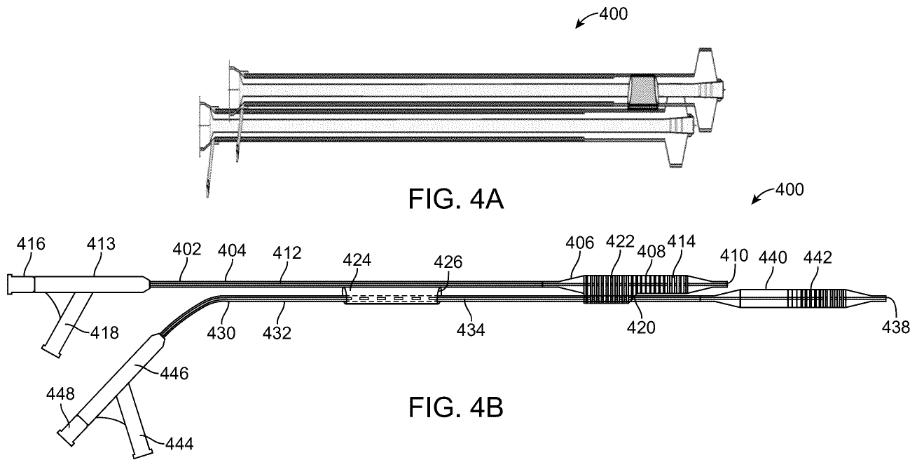

FIG. 4A illustrates a cross-sectional view of one example of a catheter system 400 with the mother and daughter catheters both having an over-the-wire design. In this particular example one of the catheters has a hollow exchange port embedded in its side and the other catheter does not have a hollow exchange port. The catheter without the exchange port is loaded onto the catheter with an exchange port. Typically, the catheter would have to be loaded prior to having a stent crimped over the balloon portion.

FIG. 4B more clearly illustrates the features of the catheter system 400 in FIG. 4A. The catheter system 400 includes a first catheter 402 and a second catheter 430. The first catheter 402 includes an elongate shaft 404 with a radially expandable balloon 406 disposed near a distal end of the elongate shaft 404. A stent 408 having a proximal portion 422, a distal portion 414 and a side hole 420 is disposed over the balloon 406. The distal portion 414 is crimped to the balloon 406 to prevent ejection during delivery, while the proximal portion 422 is partially crimped to the balloon 406 so the second catheter 430 may be slidably advanced under the proximal portion 422 of stent 408. The first catheter 402 is an over-the-wire (OTW) catheter having a guidewire lumen 412 extending from the distal guidewire port 410 at the distal end of the elongate shaft 404 to the proximal end of the elongate shaft 404 into Y-adapter 413 having a connector 416. The connector 416 I optionally a Luer connector, and this allows easy coupling with a syringe or other device for lumen flushing or injecting contrast media. When unconnected, the guidewire lumen 412 exits via connector 416. A second connector 418, also optionally a Luer connector, allows attachment of an Indeflator or other device to the catheter for inflation of the balloon 406 via an inflation lumen (not shown) in the elongate shaft 404. The first catheter 402 also includes a hollow exchange port tube 424 coupled to the elongate shaft 404. The hollow exchange port tube 424 may be coextruded with the first shaft 404, or it may be bonded or otherwise attached thereto using techniques known to those skilled in the art. The hollow exchange port tube 424 may alternatively be coupled with the other shaft 432. The hollow exchange port tube 424 includes a central channel 426 extending therethrough and is sized to slidably receive a portion of the second catheter 430. Radiopaque markers may be placed at different locations along the shaft 404, often near the balloon 406 and/or stent 408, to help mark the proximal and distal ends of the stent 408 or balloon 406, as well as to facilitate alignment of the two catheters during stent deployment, as discussed elsewhere in this specification.

The second catheter 430 includes an elongate shaft 432 with a radially expandable balloon 440 disposed near a distal end of the elongate shaft 432. A stent 442 is disposed over balloon 440. The stent 442 may have a length that matches the working length of the balloon 440, or the stent length may be shorter than the balloon working length. In some examples, the stent 442 is shorter than the working length of the balloon 440 so that a proximal portion of the balloon 440 is unconstrained by the stent 442 and this unconstrained portion of the balloon 440 may be slidably advanced or retracted through side hole 420 and under proximal portion 422 of stent 408 as will be discussed below. Stent 442 is crimped to balloon 440 to prevent ejection during delivery. At least a portion of balloon 440 and stent 442 are distally offset relative to balloon 406 and stent 408 so as to minimize profile of the device. In this example the distal stent 442 may be deployed in a main branch of the vessel and the other stent 408 may be deployed in a side branch of the vessel. Alternatively, the distal stent 442 may be deployed in a side branch of a vessel and the other stent 408 may be deployed in the main branch of a vessel.

The second catheter 430 is an over-the-wire (OTW) catheter having a guidewire lumen 434 extending from the distal guidewire port 438 at the distal end of the elongate shaft 432 to the proximal end of the elongate shaft 432 into Y-adapter 446 having a connector 448. The connector 448 may be a Luer connector, and this allows easy coupling with a syringe or other device for lumen flushing or injecting contrast media. When unconnected, the guidewire lumen 434 exits via connector 448. A second connector 444, also may be a Luer connector, allows attachment of an Indeflator or other device to the catheter for inflation of the balloon 440 via an inflation lumen (not shown) in the elongate shaft 432. A portion of shaft 432 is disposed in the central channel 426 of the hollow exchange port tube 424 and this helps keep the two catheter shafts 404, 432 parallel and prevents tangling during delivery and as shaft 432 is slidably advanced or retracted relative to shaft 404. Also, another portion of shaft 432 is disposed under proximal portion 422 of stent 408. The second catheter 430 may also be slidably advanced or retracted under the proximal portion 422 of stent 408 so that the shaft 432 passes through the side hole 420 in stent 408. Radiopaque markers may be placed at different locations on the shaft 432, often near the balloon 440 or stent 442, to help mark the proximal and distal ends of the stent 442 or balloon 440, as well as to facilitate alignment of the two catheters during stent deployment, as discussed elsewhere in this specification.

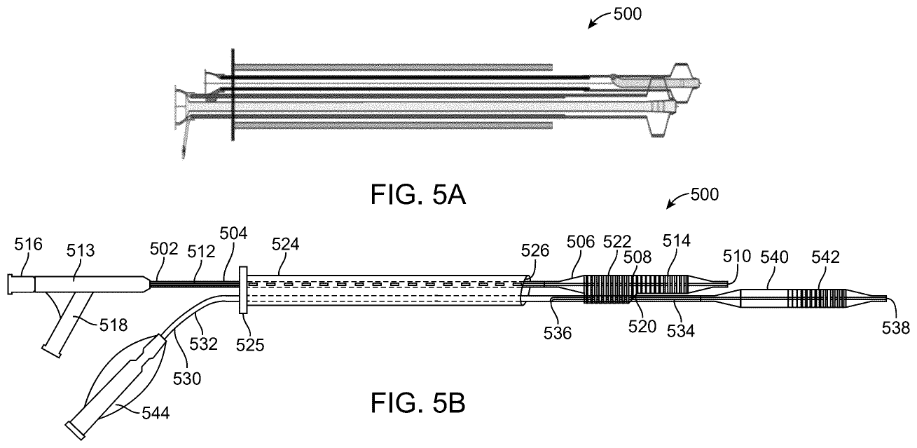

FIGS. 5A, 6A, 7A, and 8A illustrate an end-to-end capture tube that connects the catheters together. The capture tube keeps the catheters from tangling. The capture tube may remain in place during the entire clinical procedure. In these examples, the capture tube is a thin polymer hollow straw that covers the mother and daughter catheters from a point about 10 centimeters distal to the Indeflator attachment to a distal point that is about 10 centimeters proximal from the rapid exchange catheter's proximal rapid exchange port.

FIG. 5A illustrates a catheter system 500 having a distal daughter catheter with a rapid exchange configuration and a proximal mother catheter with an over-the-wire configuration. FIG. 5B more clearly illustrates the features of the catheter system 500 seen in FIG. 5A. The catheter system 500 includes a first catheter 502 and a second catheter 530. The first catheter 502 includes an elongate shaft 504 with a radially expandable balloon 506 disposed near a distal end of the elongate shaft 504. A stent 508 having a proximal portion 522 a distal portion 514 and a side hole 520 is disposed over the balloon 506. The distal portion 514 is crimped to the balloon 506 to prevent ejection during delivery, while the proximal portion 522 is partially crimped to the balloon 506 so the second catheter 530 may be slidably advanced under the proximal portion 522 of stent 508. The first catheter 502 is an over-the-wire (OTW) catheter having a guidewire lumen 512 extending from the distal guidewire port 510 at the distal end of the elongate shaft 504 to the proximal end of the elongate shaft 504 into Y-adapter 513 having a connector 516. The connector 516 may be a Luer connector, and this allows easy coupling with a syringe or other device for lumen flushing or injecting contrast media. When unconnected, the guidewire lumen 512 exits via connector 516. A second connector 518, also may be a Luer connector, allows attachment of an Indeflator or other device to the first catheter 502 for inflation of the balloon 506 via an inflation lumen (not shown) in the elongate shaft 504. The first catheter 502 is disposed in the central channel 526 of a capture tube 524. Central channel 526 is sized to fit both shafts 504, 532 and allow slidable movement thereof. Shaft 504 is slidable in the central channel 526, or it may be locked with a compression fitting such as a locking collar 525 such as a Tuohy-Borst compression fitting. Radiopaque markers may be placed at different locations along the shaft 504, often near the balloon 506 and/or stent 508, to help mark the proximal and distal ends of the stent or balloon, as well to facilitate alignment of the two catheters during stent deployment, as discussed elsewhere in this specification.

The second catheter 530 includes an elongate shaft 532 with a radially expandable balloon 540 disposed near a distal end of the elongate shaft 532. A stent 542 is disposed over balloon 540. The stent 542 may have a length that matches the working length of the balloon 540, or the stent length may be shorter than the balloon working length. In some examples, the stent 542 is shorter than the working length of the balloon 540 so that a proximal portion of the balloon 540 is unconstrained by the stent 542, and this unconstrained portion of the balloon 540 may be slidably advanced or retracted through side hole 520 and under proximal portion 522 of stent 508 as will be discussed below. Stent 542 is crimped to balloon 540 to prevent ejection during delivery. At least a portion of balloon 540 and stent 542 are distally offset relative to balloon 506 and stent 508 so as to minimize profile of the device. In this example the distal stent 542 may be deployed in a main branch of the vessel and the other stent 508 may be deployed in a side branch of the vessel. Alternatively, the distal stent 542 may be deployed in a side branch of a vessel and the other stent 508 may be deployed in the main branch of a vessel.

The second catheter 530 is a rapid exchange catheter (RX) having a guidewire lumen 534 extending from the distal guidewire port 538 at the distal end of the elongate shaft 532 to a proximal guidewire port 536, which is closer to the distal guidewire port 538 than the proximal end of the catheter shaft 532. The proximal guidewire port 536 is also unobstructed by the capture tube 524 and may be distal thereto. A connector 544, optionally a Luer connector, is connected to the proximal end of the elongate shaft 532 and allows an Indeflator or other device to be coupled with an inflation lumen (not shown) in elongate shaft 532 for inflation of balloon 540. A portion of shaft 532 is disposed in the central channel 526 of the capture tube 524, and this helps keep the two catheter shafts 504, 532 parallel and prevents tangling during delivery and as shaft 532 is slidably advanced in the central channel 526. Locking collar 525 may be used to lock elongate shafts 504, 532 in the capture tube 524 to prevent axial movement. The compression fitting may be a Tuohy-Borst fitting. Also, another portion of shaft 532 is disposed under proximal portion 522 of stent 508. The second catheter 530 may also be slidably advanced or retracted under the proximal portion 522 of stent 508 so that the shaft 532 passes through the side hole 520 in stent 508. Radiopaque markers may be placed at different locations on the shaft 532, often near the balloon 540 or stent 542, to help mark the proximal and distal ends of the stent or balloon, as well as to facilitate alignment of the two catheters during stent deployment, as discussed elsewhere in this specification.

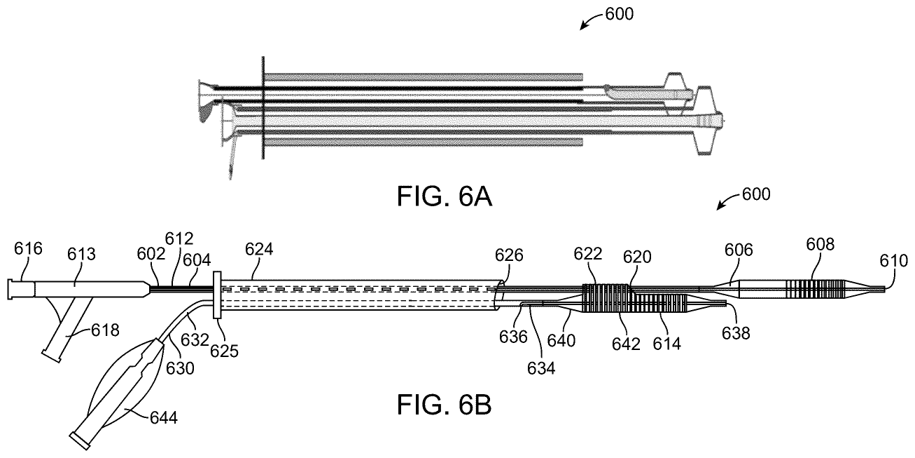

FIG. 6A illustrates a catheter system 600 having a distal daughter catheter with an over-the-wire design and a proximal mother catheter with a rapid exchange design. FIG. 6B more clearly illustrates the features of the catheter system 600 in FIG. 6A. The catheter system 600 includes a first catheter 602 and a second catheter 630. The first catheter 602 includes an elongate shaft 604 with a radially expandable balloon 606 disposed near a distal end of the elongate shaft 604, and a stent 608 disposed over the balloon 606. The stent 608 may be the same length as the working length of the balloon 606, or it may be shorter. In some examples, the stent 608 is shorter than the working length of balloon 606 such that a proximal portion of balloon 606 remains unconstrained by stent 608. The proximal portion of balloon 606 may be slidably advanced and retracted under stent 642 via side hole 620. Stent 608 is crimped to the balloon 606 to prevent ejection during delivery. The first catheter 602 is an over-the-wire (OTW) catheter having a guidewire lumen 612 extending from the distal guidewire port 610 at the distal end of the elongate shaft 604 to the proximal end of the elongate shaft 604 into Y-adapter 613 having a connector 616. The connector 616 is optionally a Luer connector, and this allows easy coupling with a syringe or other device for lumen flushing or injecting contrast media. When unconnected, the guidewire lumen 612 exits via connector 616. A second connector 618, also optionally a Luer connector, allows attachment of an Indeflator or other device to the first catheter 602 for inflation of the balloon 606 via an inflation lumen (not shown) in the elongate shaft 604. The first catheter 602 is disposed in the central channel 626 of a capture tube 624. Central channel 626 is sized to fit both shafts 604, 632 and allow slidable movement thereof. Shaft 604 is slidable in the central channel 626, or it may be locked with a locking collar 625 such as a Tuohy-Borst compression fitting. Radiopaque markers may be placed at different locations along the shaft 604, often near the balloon 606 and/or stent 608, to help mark the proximal and distal ends of the stent 608 or balloon 606, as well to facilitate alignment of the two catheters during stent deployment, as discussed elsewhere in this specification.

The second catheter 630 includes an elongate shaft 632 with a radially expandable balloon 640 disposed near a distal end of the elongate shaft 632. A stent 642 having a proximal portion 622, a distal portion 614, and a side hole 620 is disposed over balloon 640. The distal portion 614 is crimped to balloon 640 to prevent ejection during delivery, while the proximal portion 622 is partially crimped to balloon 640 so elongate shaft 604 may be slidably advanced or retracted under the proximal portion 622 of stent 642. The stent 642 may have a length that matches the working length of the balloon 640, or the stent length may be shorter than the balloon working length. At least a portion of balloon 606 and stent 608 are distally offset relative to balloon 640 and stent 642 so as to minimize the profile of the device. In this example the distal stent 608 may be deployed in a main branch of the vessel and the other stent 642 may be deployed in a side branch of the vessel. Alternatively, the distal stent 608 may be deployed in a side branch of a vessel and the other stent 642 may be deployed in the main branch of a vessel.

The second catheter 630 is a rapid exchange catheter (RX) having a guidewire lumen 634 extending from the distal guidewire port 638 at the distal end of the elongate shaft 632 to a proximal guidewire port 636, which is closer to the distal guidewire port 638 than the proximal end of the catheter shaft 632. The proximal guidewire port 636 is also unobstructed by the capture tube 624 and may be distal thereto. A connector 644, optionally a Luer connector, is connected to the proximal end of the elongate shaft 632 and allows an Indeflator or other device to be coupled with an inflation lumen (not shown) in elongate shaft 632 for inflation of balloon 640. A portion of shaft 632 is disposed in the central channel 626 of the capture tube 624 and this helps keep the two catheter shafts 604, 632 parallel and prevents tangling during delivery and as shaft 604 is slidably advanced in the central channel 626. Locking collar 625 may be used to lock elongate shafts 604, 632 in the capture tube 624 to prevent axial movement. The compression fitting may be a Tuohy-Borst fitting. Also, a portion of shaft 604 is disposed under proximal portion 622 of stent 642. The first catheter 602 may be slidably advanced or retracted under the proximal portion 622 of stent 642 so that the shaft 604 passes through the side hole 620 in stent 642. Radiopaque markers may be placed at different locations on the shaft 632, often near the balloon 640 or stent 642, to help mark the proximal and distal ends of the stent or balloon, as well to facilitate alignment of the two catheters during stent deployment, as discussed elsewhere in this specification.

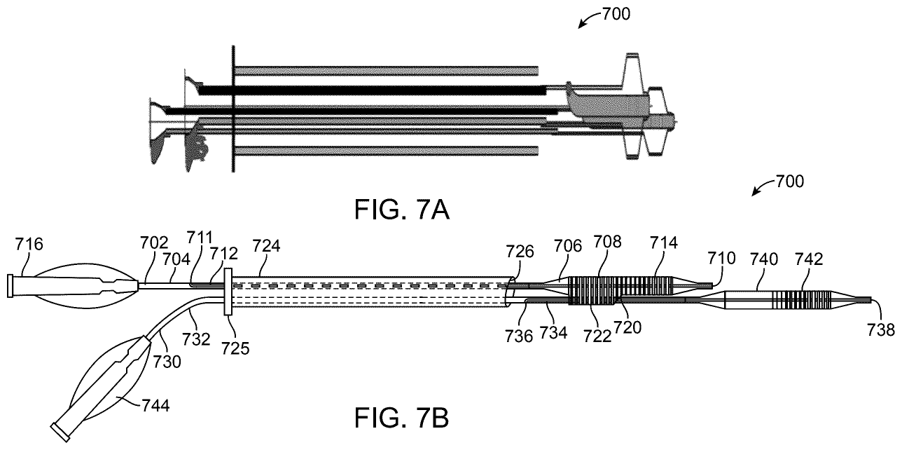

FIG. 7A shows a catheter system 700 having dual rapid exchange mother and daughter catheters so the end point of the capture tube is optionally about 10 centimeters proximal from the rapid exchange port on the distal most catheter. FIG. 7B more clearly illustrates the features of the catheter system 700 in FIG. 7A. The catheter system 700 includes a first catheter 702 and a second catheter 730. The first catheter 702 includes an elongate shaft 704 with a radially expandable balloon 706 disposed near a distal end of the elongate shaft 704. A stent 708 having a proximal portion 722, a distal portion 714 and a side hole 720 is disposed over the balloon 706. The distal portion 714 is crimped to the balloon 706 to prevent ejection during delivery, while the proximal portion 722 is partially crimped to the balloon 706 so the second catheter 730 may be slidably advanced under the proximal portion 722 of stent 708. The first catheter 702 is a rapid exchange catheter (RX) having a guidewire lumen 712 extending from the distal guidewire port 710 at the distal end of the elongate shaft 704 to a proximal guidewire port 711, which is closer to the distal guidewire port 710 than the proximal end of the catheter shaft 704. A connector 716 is coupled with the proximal end of the elongate shaft 704. The connector 716 is optionally a Luer connector, and this allows easy coupling with an Indeflator or other device for inflation of the balloon 706. The first catheter 702 is disposed in the central channel 726 of a capture tube 724. Central channel 726 is sized to fit both shafts 704, 732 and allow slidable movement thereof. Shaft 704 is slidable in the central channel 726, or it may be locked with a locking collar 725 such as a Tuohy-Borst compression fitting. Radiopaque markers may be placed at different locations along the shaft 704, often near the balloon 706 and/or stent 708, to help mark the proximal and distal ends of the stent 708 or balloon 706, as well as to facilitate alignment of the two catheters during stent deployment, as discussed elsewhere in this specification.

The second catheter 730 includes an elongate shaft 732 with a radially expandable balloon 740 disposed near a distal end of the elongate shaft 732. A stent 742 is disposed over balloon 740. The stent 742 may have a length that matches the working length of the balloon 740, or the stent length may be shorter than the balloon working length. In some examples, the stent 742 is shorter than the working length of the balloon 740 so that a proximal portion of the balloon 740 is unconstrained by the stent 742, and this unconstrained portion of the balloon 740 may be slidably advanced or retracted through side hole 720 and under proximal portion 722 of stent 708 as will be discussed below. Stent 742 is crimped to balloon 740 to prevent ejection during delivery. At least a portion of balloon 740 and stent 742 are distally offset relative to balloon 706 and stent 708 so as to minimize profile of the device. In this example the distal stent 742 may be deployed in a main branch of the vessel and the other stent 708 may be deployed in a side branch of the vessel. Alternatively, the distal stent 742 may be deployed in a side branch of a vessel and the other stent 708 may be deployed in the main branch of a vessel.

The second catheter 730 is a rapid exchange catheter (RX) having a guidewire lumen 734 extending from the distal guidewire port 738 at the distal end of the elongate shaft 732 to a proximal guidewire port 736, which is closer to the distal guidewire port 738 than the proximal end of the catheter shaft 732. The proximal guidewire port 736 is also unobstructed by the capture tube 724 and may be distal thereto. A connector 744, optionally a Luer connector, is connected to the proximal end of the elongate shaft 732 and allows an Indeflator or other device to be coupled with an inflation lumen (not shown) in elongate shaft 732 for inflation of balloon 740. A portion of shaft 732 is disposed in the central channel 726 of the capture tube 724 and this helps keep the two catheter shafts 704, 732 parallel and prevents tangling during delivery and as shaft 732 is slidably advanced in the central channel 726. Locking collar 725 may be used to lock elongate shafts 704, 732 in the capture tube 724 to prevent axial movement. The compression fitting may be a Tuohy-Borst fitting. Also, another portion of shaft 732 is disposed under proximal portion 722 of stent 708. The second catheter 730 may also be slidably advanced or retracted under the proximal portion 722 of stent 708 so that the shaft 732 passes through the side hole 720 in stent 708. Radiopaque markers may be placed at different locations on the shaft 732, often near the balloon 740 or stent 742, to help mark the proximal and distal ends of the stent 742 or balloon 740, as well as to facilitate alignment of the two catheters during stent deployment, as discussed elsewhere in this specification.

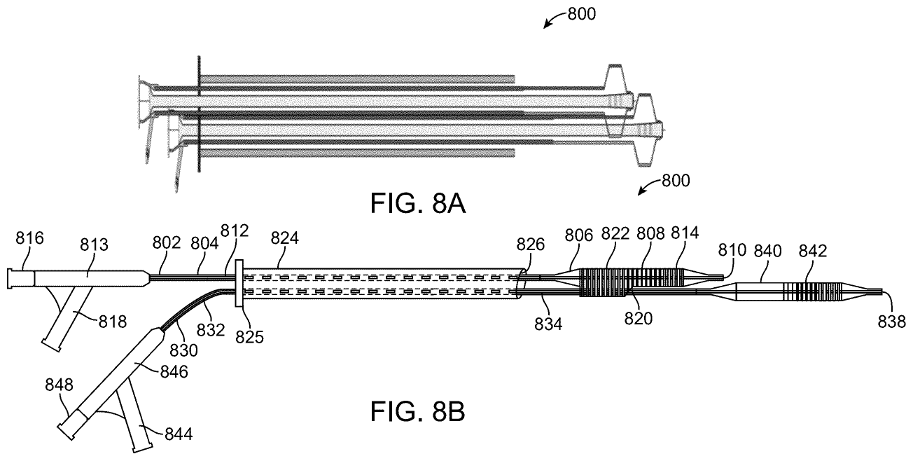

FIG. 8A illustrates a catheter system 800 with dual over-the-wire designs; therefore, the capture tube ending point ends optionally about 30 centimeters proximal from the balloon portion of the most distal catheter. FIG. 8B more clearly illustrates the features of the catheter system 800 in FIG. 8A. The catheter system 800 includes a first catheter 802 and a second catheter 830. The first catheter 802 includes an elongate shaft 804 with a radially expandable balloon 806 disposed near a distal end of the elongate shaft 804. A stent 808 having a proximal portion 822, a distal portion 814 and a side hole 820 is disposed over the balloon 806. The distal portion 814 is crimped to the balloon 806 to prevent ejection during delivery, while the proximal portion 822 is partially crimped to the balloon 806 so the second catheter 830 may be slidably advanced under the proximal portion 822 of stent 808. The first catheter 802 is an over-the-wire (OTW) catheter having a guidewire lumen 812 extending from the distal guidewire port 810 at the distal end of the elongate shaft 804 to the proximal end of the elongate shaft 804 into Y-adapter 813 having a connector 816. The connector 816 is optionally a Luer connector, and this allows easy coupling with a syringe or other device for lumen flushing or injecting contrast media. When unconnected, the guidewire lumen 812 exits via connector 816. A second connector 818, also optionally a Luer connector, allows attachment of an Indeflator or other device to the first catheter 802 for inflation of the balloon 806 via an inflation lumen (not shown) in the elongate shaft 804. The first catheter 802 is disposed in the central channel 826 of a capture tube 824. Central channel 826 is sized to fit both shafts 804, 832 and allow slidable movement thereof. Shaft 804 is slidable in the central channel 826, or it may be locked with a locking collar 825 such as a Tuohy-Borst compression fitting. Radiopaque markers may be placed at different locations along the shaft 804, often near the balloon 806 and/or stent 808, to help mark the proximal and distal ends of the stent 808 or balloon 806, as well to facilitate alignment of the two catheters during stent deployment, as discussed elsewhere in this specification.

The second catheter 830 includes an elongate shaft 832 with a radially expandable balloon 840 disposed near a distal end of the elongate shaft 832. A stent 842 is disposed over balloon 840. The stent 842 may have a length that matches the working length of the balloon 840, or the stent length may be shorter than the balloon working length. In some examples, the stent 842 is shorter than the working length of the balloon 840 so that a proximal portion of the balloon 840 is unconstrained by the stent 842, and this unconstrained portion of the balloon 840 may be slidably advanced or retracted through side hole 820 and under proximal portion 822 of stent 808 as will be discussed below. Stent 842 is crimped to balloon 840 to prevent ejection during delivery. At least a portion of balloon 840 and stent 842 are distally offset relative to balloon 806 and stent 808 so as to minimize profile of the device. In this example the distal stent 842 may be deployed in a main branch of the vessel and the other stent 808 may be deployed in a side branch of the vessel. Alternatively, the distal stent 842 may be deployed in a side branch of a vessel and the other stent 808 may be deployed in the main branch of a vessel.

The second catheter 830 is an over-the-wire (OTW) catheter having a guidewire lumen 834 extending from the distal guidewire port 838 at the distal end of the elongate shaft 832 to the proximal end of the elongate shaft 832 into Y-adapter 846 having a connector 848. The connector 848 is optionally a Luer connector, and this allows easy coupling with a syringe or other device for lumen flushing or injecting contrast media. When unconnected, the guidewire lumen 834 exits via connector 848. A second connector 844, also optionally a Luer connector, allows attachment of an Indeflator or other device to the catheter for inflation of the balloon 840 via an inflation lumen (not shown) in the elongate shaft 832. A portion of shaft 832 is disposed in the central channel 826 of the capture tube 824, and this helps keep the two catheter shafts 804, 832 parallel and prevents tangling during delivery and as shaft 832 is slidably advanced in the central channel 826. Locking collar 825 may be used to lock elongate shafts 804, 832 in the capture tube 824 to prevent axial movement. The compression fitting may be a Tuohy-Borst fitting. Also, another portion of shaft 832 is disposed under proximal portion 822 of stent 808. The second catheter 830 may also be slidably advanced or retracted under the proximal portion 822 of stent 808 so that the shaft 832 passes through the side hole 820 in stent 808. Radiopaque markers may be placed at different locations on the shaft 832, often near the balloon 840 or stent 842, to help mark the proximal and distal ends of the stent 842 or balloon 840, as well as to facilitate alignment of the two catheters during stent deployment, as discussed elsewhere in this specification.

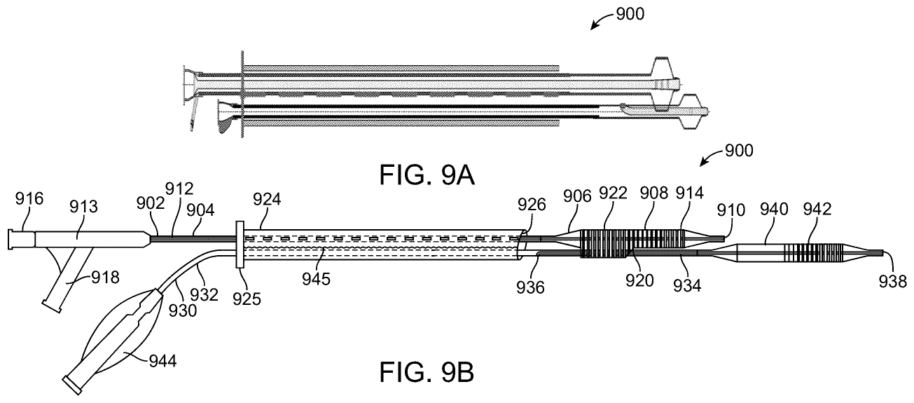

FIGS. 9A, 10A, 11A, and 12A illustrate a removable capture tube that is fitted over the dual catheters as described above, but the capture tube has a polymer appendage. Once the operator has the catheter system placed near the bifurcation, the operator can grab hold of the polymer appendage and pull the capture tube off of the catheters.

FIG. 9A illustrates a catheter system 900 having a distal daughter catheter with a rapid exchange configuration and a proximal mother catheter with an over-the-wire configuration. FIG. 9B more clearly illustrates the features of the catheter system 900 seen in FIG. 9A. The catheter system 900 includes a first catheter 902 and a second catheter 930. The first catheter 902 includes an elongate shaft 904 with a radially expandable balloon 906 disposed near a distal end of the elongate shaft 904. A stent 908 having a proximal portion 922, a distal portion 914 and a side hole 920 is disposed over the balloon 906. The distal portion 914 is crimped to the balloon 906 to prevent ejection during delivery, while the proximal portion 922 is partially crimped to the balloon 906 so the second catheter 930 may be slidably advanced under the proximal portion 922 of stent 908. The first catheter is an over-the-wire (OTW) catheter having a guidewire lumen 912 extending from the distal guidewire port 910 at the distal end of the elongate shaft 904 to the proximal end of the elongate shaft 904 into Y-adapter 913 having a connector 916. The connector 916 is optionally a Luer connector, and this allows easy coupling with a syringe or other device for lumen flushing or injecting contrast media. When unconnected, the guidewire lumen 912 exits via connector 916. A second connector 918, also optionally a Luer connector, allows attachment of an Indeflator or other device to the catheter for inflation of the balloon 906 via an inflation lumen (not shown) in the elongate shaft 904. The first catheter 902 is disposed in the central channel 926 of a capture tube 924 having a perforated region 945 along its longitudinal length. Central channel 926 is sized to fit both shafts 904, 932 and allow slidable movement thereof. Shaft 904 is slidable in the central channel 926, or it may be locked with a locking collar 925 such as a Tuohy-Borst compression fitting. Radiopaque markers may be placed at different locations along the shaft 904, often near the balloon 906 and/or stent 908, to help mark the proximal and distal ends of the stent 908 or balloon 906, as well to facilitate alignment of the two catheters during stent deployment, as discussed elsewhere in this specification. The perforated region 945 along the capture tube 924 allows the capture tube 924 to be easily peeled away from both catheter shafts 904, 932 once the catheters have been properly positioned and when no longer needed.

The second catheter 930 includes an elongate shaft 932 with a radially expandable balloon 940 disposed near a distal end of the elongate shaft 932. A stent 942 is disposed over balloon 940. The stent 942 may have a length that matches the working length of the balloon 940, or the stent length may be shorter than the balloon working length. In some examples, the stent 942 is shorter than the working length of the balloon 940 so that a proximal portion of the balloon 940 is unconstrained by the stent 942, and this unconstrained portion of the balloon 940 may be slidably advanced or retracted through side hole 920 and under proximal portion 922 of stent 908 as will be discussed below. Stent 942 is crimped to balloon 940 to prevent ejection during delivery. At least a portion of balloon 940 and stent 942 are distally offset relative to balloon 906 and stent 908 so as to minimize profile of the device. In this example the distal stent 942 may be deployed in a main branch of the vessel and the other stent 908 may be deployed in a side branch of the vessel. Alternatively, the distal stent 942 may be deployed in a side branch of a vessel and the other stent 908 may be deployed in the main branch of a vessel.

The second catheter 930 is a rapid exchange catheter (RX) having a guidewire lumen 934 extending from the distal guidewire port 938 at the distal end of the elongate shaft 932 to a proximal guidewire port 936, which is closer to the distal guidewire port 938 than the proximal end of the catheter shaft 932. The proximal guidewire port 936 is also unobstructed by the capture tube 924 and may be distal thereto. A connector 944, optionally a Luer connector, is connected to the proximal end of the elongate shaft 932 and allows an Indeflator or other device to be coupled with an inflation lumen (not shown) in elongate shaft 932 for inflation of balloon 940. A portion of shaft 932 is disposed in the central channel 926 of the capture tube 924 and this helps keep the two catheter shafts 904, 932 parallel and prevents tangling during delivery and as shaft 932 is slidably advanced in the central channel 926. Locking collar 925 may be used to lock elongate shafts 904, 932 in the capture tube 924 to prevent axial movement. The compression fitting may be a Tuohy-Borst fitting. Also, another portion of shaft 932 is disposed under proximal portion 922 of stent 908. The second catheter 930 may also be slidably advanced or retracted under the proximal portion 922 of stent 908 so that the shaft 932 passes through the side hole 920 in stent 908. Capture tube 924 may be peeled away from shaft 932 by severing the perforated region 945. Radiopaque markers may be placed at different locations on the shaft 932, often near the balloon 940 or stent 942, to help mark the proximal and distal ends of the stent 942 or balloon 940, as well to facilitate alignment of the two catheters during stent deployment, as discussed elsewhere in this specification.

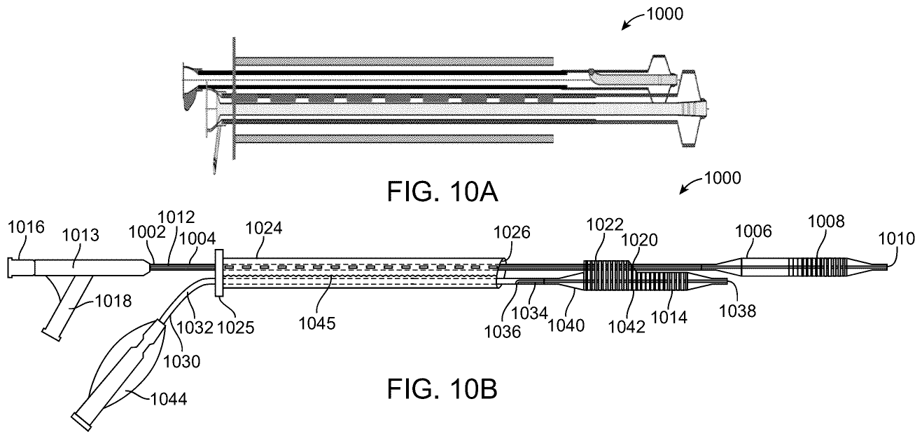

FIG. 10A illustrates a catheter system 1000 having a distal daughter catheter with an over-the-wire design and a proximal mother catheter with a rapid exchange design. FIG. 10B more clearly illustrates the features of the catheter system 1000 in FIG. 10A. The catheter system 1000 includes a first catheter 1002 and a second catheter 1030. The first catheter 1002 includes an elongate shaft 1004 with a radially expandable balloon 1006 disposed near a distal end of the elongate shaft 1004, and a stent 1008 disposed over the balloon 1006. The stent 1008 may be the same length as the working length of the balloon 1006, or it may be shorter. In some examples, the stent 1008 is shorter than the working length of balloon 1006 such that a proximal portion of balloon 1006 remains unconstrained by stent 1008. The proximal portion of balloon 1006 may be slidably advanced and retracted under stent 1042 via side hole 1020. Stent 1008 is crimped to the balloon 1006 to prevent ejection during delivery. The first catheter 1002 is an over-the-wire (OTW) catheter having a guidewire lumen 1012 extending from the distal guidewire port 1010 at the distal end of the elongate shaft 1004 to the proximal end of the elongate shaft 1004 into Y-adapter 1013 having a connector 1016. The connector 1016 is optionally a Luer connector, and this allows easy coupling with a syringe or other device for lumen flushing or injecting contrast media. When unconnected, the guidewire lumen 1012 exits via connector 1016. A second connector 1018, also optionally a Luer connector, allows attachment of an Indeflator or other device to the catheter for inflation of the balloon 1006 via an inflation lumen (not shown) in the elongate shaft 1004. The first catheter 1002 is disposed in the central channel 1026 of a capture tube 1024 having perforated region 1045. Central channel 1026 is sized to fit both shafts 1004, 1032 and allow slidable movement thereof. Shaft 1004 is slidable in the central channel 1026, or it may be locked with a locking collar 1025 such as a Tuohy-Borst compression fitting. Radiopaque markers may be placed at different locations along the shaft 1004, often near the balloon 1006 and/or stent 1008, to help mark the proximal and distal ends of the stent 1008 or balloon 1006, as well to facilitate alignment of the two catheters during stent deployment, as discussed elsewhere in this specification. The perforated region 1045 along the capture tube 1024 allows the capture tube 1024 to be easily peeled away from both catheter shafts 1004, 1032 once the catheters have been properly positioned and when no longer needed.

The second catheter 1030 includes an elongate shaft 1032 with a radially expandable balloon 1040 disposed near a distal end of the elongate shaft 1032. A stent 1042 having a proximal portion 1022, a distal portion 1014, and a side hole 1020 is disposed over balloon 1040. The distal portion 1014 is crimped to balloon 1040 to prevent ejection during delivery, while the proximal portion 1022 is partially crimped to balloon 1040 so elongate shaft 1004 may be slidably advanced or retracted under the proximal portion 1022 of stent 1042. The stent 1042 may optionally have a length that matches the working length of the balloon 1040, or the stent length may be shorter than the balloon working length. At least a portion of balloon 1006 and stent 1008 are distally offset relative to balloon 1040 and stent 1042 so as to minimize profile of the device. In this example the distal stent 1008 may be deployed in a main branch of the vessel and the other stent 1042 may be deployed in a side branch of the vessel. Alternatively, the distal stent 1008 may be deployed in a side branch of a vessel and the other stent 1042 may be deployed in the main branch of a vessel.

The second catheter 1030 is a rapid exchange catheter (RX) having a guidewire lumen 1034 extending from the distal guidewire port 1038 at the distal end of the elongate shaft 1032 to a proximal guidewire port 1036, which is closer to the distal guidewire port 1038 than the proximal end of the catheter shaft 1032. The proximal guidewire port 1036 is also unobstructed by the capture tube 1024 and may be distal thereto. A connector 1044, optionally a Luer connector, is connected to the proximal end of the elongate shaft 1032 and allows an Indeflator or other device to be coupled with an inflation lumen (not shown) in elongate shaft 1032 for inflation of balloon 1040. A portion of shaft 1032 is disposed in the central channel 1026 of the capture tube 1024 and this helps keep the two catheter shafts 1004, 1032 parallel and prevents tangling during delivery and as shaft 1032 is slidably advanced in the central channel 1026. Locking collar 1025 may be used to lock elongate shafts 1004, 1032 in the capture tube 1024 to prevent axial movement. The compression fitting may be a Tuohy-Borst fitting. Also, a portion of shaft 1004 is disposed under proximal portion 1022 of stent 1042. The first catheter 1002 may be slidably advanced or retracted under the proximal portion 1022 of stent 1042 so that the shaft 1004 passes through the side hole 1020 in stent 1042. Capture tube 1024 may be peeled away from shaft 1032 by severing the perforated region 1045. Radiopaque markers may be placed at different locations on the shaft 1032, often near the balloon 1040 or stent 1042, to help mark the proximal and distal ends of the stent 1042 or balloon 1040, as well as to facilitate alignment of the two catheters during stent deployment, as discussed elsewhere in this specification.

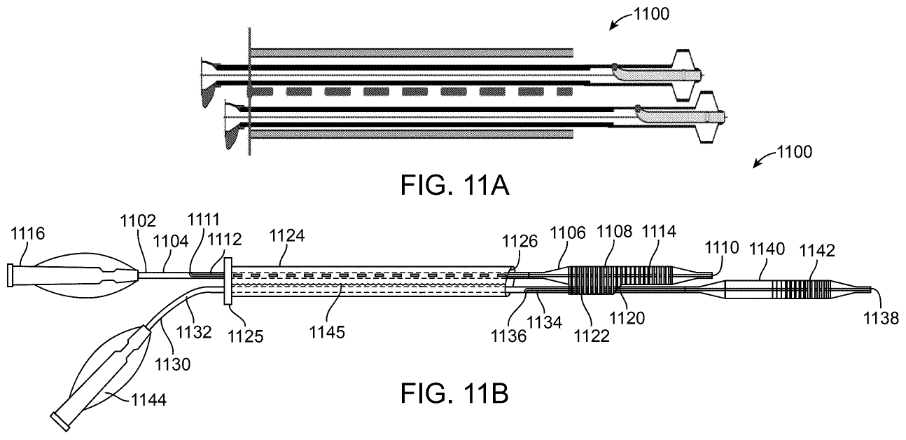

FIG. 11A illustrates a catheter system 1100 having dual rapid exchange design with a removable capture tube. FIG. 11B more clearly illustrates the features of the catheter system 1100 in FIG. 11A. The catheter system 1100 includes a first catheter 1102 and a second catheter 1130. The first catheter 1102 includes an elongate shaft 1104 with a radially expandable balloon 1106 disposed near a distal end of the elongate shaft 1104. A stent 1108 having a proximal portion 1122, a distal portion 1114 and a side hole 1120 is disposed over the balloon 1106. The distal portion 1114 is crimped to the balloon 1106 to prevent ejection during delivery, while the proximal portion 1122 is partially crimped to the balloon 1106 so the second catheter 1130 may be slidably advanced under the proximal portion 1122 of stent 1108. The first catheter 1102 is a rapid exchange catheter (RX) having a guidewire lumen 1112 extending from the distal guidewire port 1110 at the distal end of the elongate shaft 1104 to a proximal guidewire port 1111, which is closer to the distal guidewire port 1110 than the proximal end of the catheter shaft 1104. A connector 1116 is coupled with the proximal end of the elongate shaft 1104. The connector 1116 may be a Luer connector, and this allows easy coupling with an Indeflator or other device for inflation of the balloon 1106. The first catheter 1102 is disposed in the central channel 1126 of a capture tube 1124 having a perforated region 1145. Central channel 1126 is sized to fit both shafts 1104, 1132 and allow slidable movement thereof. Shaft 1104 is slidable in the central channel 1126, or it may be locked with a locking collar 1125 such as a Tuohy-Borst compression fitting. Radiopaque markers may be placed at different locations along the shaft 1104, often near the balloon 1106 and/or stent 1108, to help mark the proximal and distal ends of the stent 1108 or balloon 1106, as well to facilitate alignment of the two catheters during stent deployment, as discussed elsewhere in this specification. The perforated region 1145 along the capture tube 1124 allows the capture tube 1124 to be easily peeled away from both catheter shafts 1104, 1132 once the catheters have been properly positioned and when no longer needed.

The second catheter 1130 includes an elongate shaft 1132 with a radially expandable balloon 1140 disposed near a distal end of the elongate shaft 1132. A stent 1142 is disposed over balloon 1140. The stent 1142 may have a length that matches the working length of the balloon 1140, or the stent length may be shorter than the balloon working length. In some examples, the stent 1142 is shorter than the working length of the balloon 1140 so that a proximal portion of the balloon 1140 is unconstrained by the stent 1142, and this unconstrained portion of the balloon 1140 may be slidably advanced or retracted through side hole 1120 and under proximal portion 1122 of stent 1108 as will be discussed below. Stent 1142 is crimped to balloon 1140 to prevent ejection during delivery. At least a portion of balloon 1140 and stent 1142 are distally offset relative to balloon 1106 and stent 1108 so as to minimize profile of the device. In this example the distal stent 1142 may be deployed in a main branch of the vessel and the other stent 1108 may be deployed in a side branch of the vessel. Alternatively, the distal stent 1142 may be deployed in a side branch of a vessel and the other stent 1108 may be deployed in the main branch of a vessel.

The second catheter 1130 is a rapid exchange catheter (RX) having a guidewire lumen 1134 extending from the distal guidewire port 1138 at the distal end of the elongate shaft 1132 to a proximal guidewire port 1136, which is closer to the distal guidewire port 1138 than the proximal end of the catheter shaft 1132. The proximal guidewire port 1136 is also unobstructed by the capture tube 1124 and may be distal thereto. A connector 1144, optionally a Luer connector, is connected to the proximal end of the elongate shaft 1132 and allows an Indeflator or other device to be coupled with an inflation lumen (not shown) in elongate shaft 1132 for inflation of balloon 1140. A portion of shaft 1132 is disposed in the central channel 1126 of the capture tube 1124 and this helps keep the two catheter shafts 1104, 1132 parallel and prevents tangling during delivery and as shaft 1132 is slidably advanced in the central channel 1126. Locking collar 1125 may be used to lock elongate shafts 1104, 1132 in the capture tube 1124 to prevent axial movement. The compression fitting may be a Tuohy-Borst fitting. Also, another portion of shaft 1132 is disposed under proximal portion 1122 of stent 1108. The second catheter 1130 may also be slidably advanced or retracted under the proximal portion 1122 of stent 1108 so that the shaft 1132 passes through the side hole 1120 in stent 1108. Capture tube 1124 may be peeled away from shaft 1132 by severing the perforated region 1145. Radiopaque markers may be placed at different locations on the shaft 1132, often near the balloon 1140 or stent 1142, to help mark the proximal and distal ends of the stent 1142 or balloon 1140, as well to facilitate alignment of the two catheters during stent deployment, as discussed elsewhere in this specification.

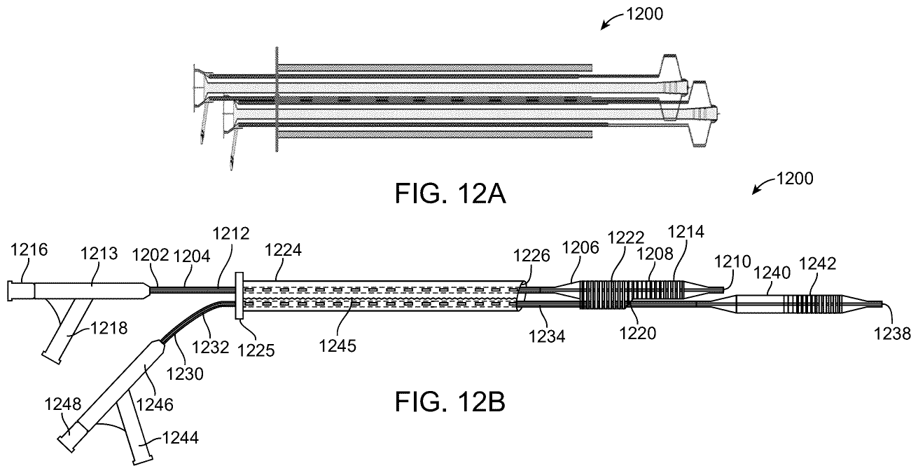

FIG. 12A illustrates a catheter system 1200 having dual over-the-wire design with a removable capture tube. FIG. 12B more clearly illustrates the features of the catheter system 1200 in FIG. 12A. The catheter system 1200 includes a first catheter 1202 and a second catheter 1230. The first catheter 1202 includes an elongate shaft 1204 with a radially expandable balloon 1206 disposed near a distal end of the elongate shaft 1204. A stent 1208 having a proximal portion 1222, a distal portion 1214 and a side hole 1220 is disposed over the balloon 1206. The distal portion 1214 is crimped to the balloon 1206 to prevent ejection during delivery, while the proximal portion 1222 is partially crimped to the balloon 1206 so the second catheter 1230 may be slidably advanced under the proximal portion 1222 of stent 1208. The first catheter 1202 is an over-the-wire (OTW) catheter having a guidewire lumen 1212 extending from the distal guidewire port 1210 at the distal end of the elongate shaft 1204 to the proximal end of the elongate shaft 1204 into Y-adapter 1213 having a connector 1216. The connector 1216 is optionally a Luer connector, and this allows easy coupling with a syringe or other device for lumen flushing or injecting contrast media. When unconnected, the guidewire lumen 1212 exits via connector 1216. A second connector 1218, also optionally a Luer connector, allows attachment of an Indeflator or other device to the first catheter 1202 for inflation of the balloon 1206 via an inflation lumen (not shown) in the elongate shaft 1204. The first catheter 1202 is disposed in the central channel 1226 of a capture tube 1224 having a perforated region 1245. Central channel 1226 is sized to fit both shafts 1204, 1232 and allow slidable movement thereof. Shaft 1204 is slidable in the central channel 1226, or it may be locked with a locking collar 1225 such as a Tuohy-Borst compression fitting. Radiopaque markers may be placed at different locations along the shaft 1204, often near the balloon 1206 and/or stent 1208, to help mark the proximal and distal ends of the stent 1208 or balloon 1206, as well to facilitate alignment of the two catheters during stent deployment, as discussed elsewhere in this specification. The perforated region 1245 along the capture tube 1224 allows the capture tube 1224 to be easily peeled away from both catheter shafts 1204, 1232 once the catheters have been properly positioned and when no longer needed.

The second catheter 1230 includes an elongate shaft 1232 with a radially expandable balloon 1240 disposed near a distal end of the elongate shaft 1232. A stent 1242 is disposed over balloon 1240. The stent 1242 may have a length that matches the working length of the balloon 1240, or the stent length may be shorter than the balloon working length. In some examples, the stent 1242 is shorter than the working length of the balloon 1240 so that a proximal portion of the balloon 1240 is unconstrained by the stent 1242, and this unconstrained portion of the balloon 1240 may be slidably advanced or retracted through side hole 1220 and under proximal portion 1222 of stent 1208 as will be discussed below. Stent 1242 is crimped to balloon 1240 to prevent ejection during delivery. At least a portion of balloon 1240 and stent 1242 are distally offset relative to balloon 1206 and stent 1208 so as to minimize profile of the device. In this example the distal stent 1242 may be deployed in a main branch of the vessel and the other stent 1208 may be deployed in a side branch of the vessel. Alternatively, the distal stent 1242 may be deployed in a side branch of a vessel and the other stent 1208 may be deployed in the main branch of a vessel.

The second catheter 1230 is an over-the-wire (OTW) catheter having a guidewire lumen 1234 extending from the distal guidewire port 1238 at the distal end of the elongate shaft 1232 to the proximal end of the elongate shaft 1232 into Y-adapter 1246 having a connector 1248. The connector 1248 is optionally a Luer connector, and this allows easy coupling with a syringe or other device for lumen flushing or injecting contrast media. When unconnected, the guidewire lumen 1234 exits via connector 1248. A second connector 1244, also optionally a Luer connector, allows attachment of an Indeflator or other device to the second catheter 1230 for inflation of the balloon 1240 via an inflation lumen (not shown) in the elongate shaft 1232. A portion of shaft 1232 is disposed in the central channel 1226 of the capture tube 1224 and this helps keep the two catheter shafts 1204, 1232 parallel and prevents tangling during delivery and as shaft 1232 is slidably advanced in the central channel 1226. Locking collar 1225 may be used to lock elongate shafts 1204, 1232 in the capture tube 1224 to prevent axial movement. The compression fitting may be a Tuohy-Borst fitting. Also, another portion of shaft 1232 is disposed under proximal portion 1222 of stent 1208. The second catheter 1230 may also be slidably advanced or retracted under the proximal portion 1222 of stent 1208 so that the shaft 1232 passes through the side hole 1220 in stent 1208. Capture tube 1224 may be peeled away from shaft 1232 by severing the perforated region 1245. Radiopaque markers may be placed at different locations on the shaft 1232, often near the balloon 1240 or stent 1242, to help mark the proximal and distal ends of the stent 1242 or balloon 1240, as well to facilitate alignment of the two catheters during stent deployment, as discussed elsewhere in this specification.

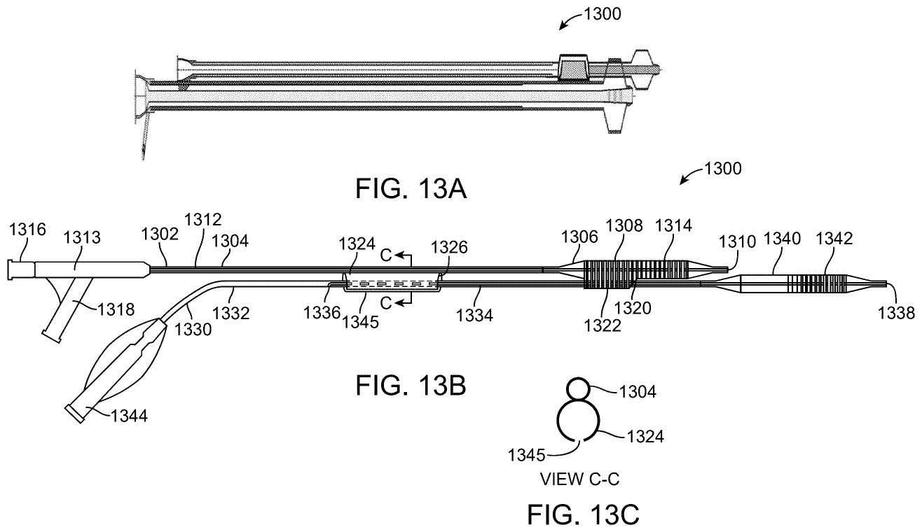

FIGS. 13A, 14A, 15A, and 16A illustrate a zipper that allows one catheter to snap into the other catheter. The zipper is essentially a groove that forms a concave receiving cross-section and is carved into a catheter's outer surface in a straight line. The groove can be a single groove over a certain portion of a catheter or it can run from end to end. Alternatively, the catheter can have a series of short grooves of 1 to 10 centimeters in length that run the length of the catheter or only a certain portion. Full length end-to-end zippers will have reduced profile and reduced friction with the vessel. The resulting groove can receive another catheter and prevent the catheters from dislodging while the operator is advancing the catheters to the bifurcation. Once at the site, the operator can still slidably move the catheters forward and back relative to each other. Mother catheters that utilize the groove can have fully crimped stents as described in several of the examples above; however, it is possible to allow operators to choose any commercially available catheter with or without a stent and mount the commercially available catheter via the zipper. The mother catheters with an empty zipper would have a mother stent fully crimped on the distal balloon portion. After loading the commercially available catheter, the operator would have to crimp the proximal portion of the mother stent in situ prior to beginning the clinical procedure. This option may be extremely valuable to operators who can reduce their total inventory of catheters but have more options for treating bifurcated lesions.

FIG. 13A illustrates a catheter system 1300 having a distal daughter catheter with an over-the-wire design and a proximal mother catheter with a rapid exchange design and a short zipper. FIG. 13B more clearly illustrates the features of the catheter system 1300 in FIG. 13A. The catheter system 1300 includes a first catheter 1302 and a second catheter 1330. The first catheter 1302 includes an elongate shaft 1304 with a radially expandable balloon 1306 disposed near a distal end of the elongate shaft 1304. A stent 1308 having a proximal portion 1322, a distal portion 1314 and a side hole 1320 is disposed over the balloon 1306. The distal portion 1314 is crimped to the balloon 1306 to prevent ejection during delivery, while the proximal portion 1322 is partially crimped to the balloon 1306 so the second catheter 1330 may be slidably advanced under the proximal portion 1322 of stent 1308. The first catheter is an over-the-wire (OTW) catheter having a guidewire lumen 1312 extending from the distal guidewire port 1310 at the distal end of the elongate shaft 1304 to the proximal end of the elongate shaft 1304 into Y-adapter 1313 having a connector 1316. The connector 1316 is optionally a Luer connector, and this allows easy coupling with a syringe or other device for lumen flushing or injecting contrast media. When unconnected, the guidewire lumen 1312 exits via connector 1316. A second connector 1318, also optionally a Luer connector, allows attachment of an Indeflator or other device to the first catheter 1302 for inflation of the balloon 1306 via an inflation lumen (not shown) in the elongate shaft 1304. The first catheter 1302 also includes a zipper or snap fitting 1324 coupled to the elongate shaft 1304. The snap fitting 1324 may be coextruded with the first shaft 1304, or it may be bonded or otherwise attached thereto using techniques known to those skilled in the art. The snap fitting 1324 may alternatively be coupled with the other shaft 1332. The snap fitting 1324 includes a central channel 1326 extending therethrough and is sized to slidably receive a portion of the second catheter 1330. An elongate slot 1345 extends along the entire length of the snap fitting 1324 and is sized so that shaft 1332 may snapped into the central channel 1326. FIG. 13C illustrates a partial cross-section of FIG. 13B taken along the line C-C and shows shaft 1304 with the snap fitting 1324. Radiopaque markers may be placed at different locations along the shaft 1304, often near the balloon 1306 and/or stent 1308, to help mark the proximal and distal ends of the stent 1308 or balloon 1306, as well as to facilitate alignment of the two catheters during stent deployment, as discussed elsewhere in this specification.

The second catheter 1330 includes an elongate shaft 1332 with a radially expandable balloon 1340 disposed near a distal end of the elongate shaft 1332. A stent 1342 is disposed over balloon 1340. The stent 1342 may have a length that matches the working length of the balloon 1340, or the stent length may be shorter than the balloon working length. In some examples, the stent 1342 is shorter than the working length of the balloon 1340 so that a proximal portion of the balloon 1340 is unconstrained by the stent 1342, and this unconstrained portion of the balloon 1340 may be slidably advanced or retracted through side hole 1320 and under proximal portion 1322 of stent 1308 as will be discussed below. Stent 1342 is crimped to balloon 1340 to prevent ejection during delivery. At least a portion of balloon 1340 and stent 1342 are distally offset relative to balloon 1306 and stent 1308 so as to minimize profile of the device. In this example the distal stent 1342 may be deployed in a main branch of the vessel and the other stent 1308 may be deployed in a side branch of the vessel. Alternatively, the distal stent 1342 may be deployed in a side branch of a vessel and the other stent 1308 may be deployed in the main branch of a vessel.

The second catheter 1330 is a rapid exchange catheter (RX) having a guidewire lumen 1334 extending from the distal guidewire port 1338 at the distal end of the elongate shaft 1332 to a proximal guidewire port 1336, which is closer to the distal guidewire port 1338 than the proximal end of the catheter shaft 1332. The proximal guidewire port 1336 is also unobstructed by the snap fitting 1324 and optionally proximal thereto. A connector 1344, optionally a Luer connector, is connected to the proximal end of the elongate shaft 1332 and allows an Indeflator or other device to be coupled with an inflation lumen (not shown) in elongate shaft 1332 for inflation of balloon 1340. A portion of shaft 1332 is snapped into the central channel 1326 of the snap fitting 1324 via slot 1345, and thus shaft 1332 may slide in channel 1326. This helps keep the two catheter shafts 1304, 1332 parallel and prevents tangling during delivery and as shaft 1332 is slidably advanced or retracted relative to shaft 1304. Also, another portion of shaft 1332 is disposed under proximal portion 1322 of stent 1308. The second catheter 1330 may also be slidably advanced or retracted under the proximal portion 1322 of stent 1308 so that the shaft 1332 passes through the side hole 1320 in stent 1308. Radiopaque markers may be placed at different locations on the shaft 1332, often near the balloon 1340 or stent 1342, to help mark the proximal and distal ends of the stent 1342 or balloon 1340, as well as to facilitate alignment of the two catheters during stent deployment, as discussed elsewhere in this specification.

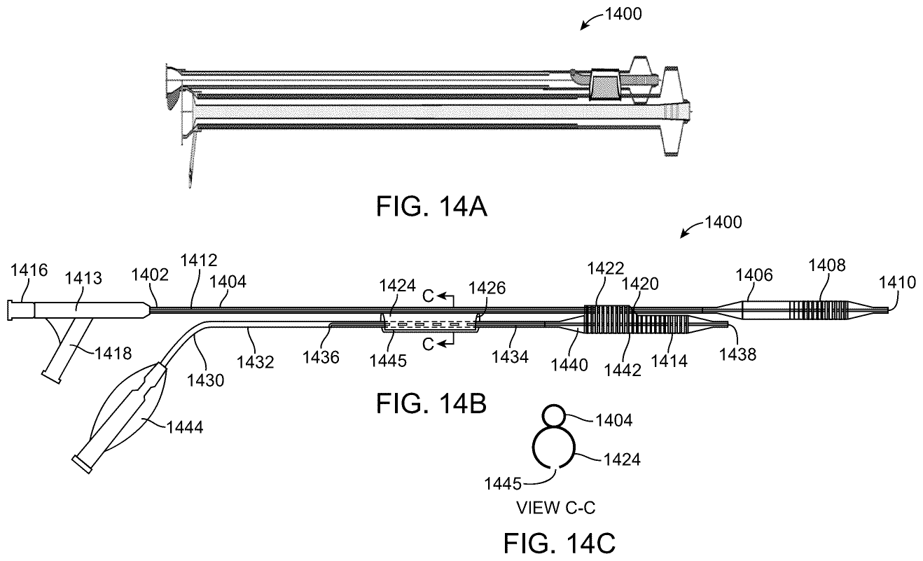

FIG. 14A illustrates a catheter system 1400 having a proximal mother catheter with a rapid exchange configuration and a distal daughter catheter having an over-the-wire configuration and a short zipper or snap fitting. FIG. 14B more clearly illustrates the features of the catheter system 1400 in FIG. 14A. The catheter system 1400 includes a first catheter 1402 and a second catheter 1430. The first catheter 1402 includes an elongate shaft 1404 with a radially expandable balloon 1406 disposed near a distal end of the elongate shaft 1404, and a stent 1408 disposed over the balloon 1406. The stent 1408 may be the same length as the working length of the balloon 1406, or it may be shorter. In some examples, the stent 1408 is shorter than the working length of balloon 1406 such that a proximal portion of balloon 1406 remains unconstrained by stent 1408. The proximal portion of balloon 1406 may be slidably advanced and retracted under stent 1442 via side hole 1420. Stent 1408 is crimped to the balloon 1406 to prevent ejection during delivery. The first catheter 1402 is an over-the-wire (OTW) catheter having a guidewire lumen 1412 extending from the distal guidewire port 1410 at the distal end of the elongate shaft 1404 to the proximal end of the elongate shaft 1404 into Y-adapter 1413 having a connector 1416. The connector 1416 is optionally a Luer connector, and this allows easy coupling with a syringe or other device for lumen flushing or injecting contrast media. When unconnected, the guidewire lumen 1412 exits via connector 1416. A second connector 1418, also optionally a Luer connector, allows attachment of an Indeflator or other device to the first catheter 1402 for inflation of the balloon 1406 via an inflation lumen (not shown) in the elongate shaft 1404. The first catheter 1402 also includes a zipper or snap fitting 1424 coupled to the elongate shaft 1404. The snap fitting 1424 may be coextruded with the first shaft 1404, or it may be bonded or otherwise attached thereto using techniques known to those skilled in the art. The snap fitting 1424 may alternatively be coupled with the other shaft 1432. The snap fitting 1424 includes a central channel 1426 extending therethrough and is sized to slidably receive a portion of the second catheter 1430. An elongate slot 1445 extends along the entire length of the snap fitting 1424 and is sized so that shaft 1432 may be snapped into the central channel 1426. FIG. 14C illustrates a partial cross-section of FIG. 14B taken along the line C-C and shows shaft 1404 with the snap fitting 1424. Radiopaque markers may be placed at different locations along the shaft 1404, often near the balloon 1406 and/or stent 1408, to help mark the proximal and distal ends of the stent 1408 or balloon 1406, as well as to facilitate alignment of the two catheters during stent deployment, as discussed elsewhere in this specification.

The second catheter 1430 includes an elongate shaft 1432 with a radially expandable balloon 1440 disposed near a distal end of the elongate shaft 1432. A stent 1442 having a proximal portion 1422, a distal portion 1414, and a side hole 1420 is disposed over balloon 1440. The distal portion 1414 is crimped to balloon 1440 to prevent ejection during delivery, while the proximal portion 1422 is partially crimped to balloon 1440 so elongate shaft 1404 may be slidably advanced or retracted under the proximal portion 1422 of stent 1442. The stent 1442 may optionally have a length that matches the working length of the balloon 1440, or the stent length may be shorter than the balloon working length. At least a portion of balloon 1406 and stent 1408 are distally offset relative to balloon 1440 and stent 1442 so as to minimize profile of the device. In this example the distal stent 1408 may be deployed in a main branch of the vessel and the other stent 1442 may be deployed in a side branch of the vessel. Alternatively, the distal stent 1408 may be deployed in a side branch of a vessel and the other stent 1442 may be deployed in the main branch of a vessel.

The second catheter 1430 is a rapid exchange catheter (RX) having a guidewire lumen 1434 extending from the distal guidewire port 1438 at the distal end of the elongate shaft 1432 to a proximal guidewire port 1436, which is closer to the distal guidewire port 1438 than the proximal end of the catheter shaft 1432. The proximal guidewire port 1436 is also unobstructed by the snap fitting 1424 and optionally proximal thereto. A connector 1444, optionally a Luer connector, is connected to the proximal end of the elongate shaft 1432 and allows an Indeflator or other device to be coupled with an inflation lumen (not shown) in elongate shaft 1432 for inflation of balloon 1440. A portion of shaft 1432 is snapped into the central channel 1426 of the snap fitting 1424 via slot 1445, and thus shaft 1432 may slide in channel 1426. This helps keep the two catheter shafts 1404, 1432 parallel and prevents tangling during delivery and as shaft 1432 is slidably advanced or retracted relative to shaft 1404. Also, a portion of shaft 1404 is disposed under proximal portion 1422 of stent 1442. The first catheter 1402 may be slidably advanced or retracted under the proximal portion 1422 of stent 1442 so that the shaft 1404 passes through the side hole 1420 in stent 1442. Radiopaque markers may be placed at different locations on the shaft 1432, often near the balloon 1440 or stent 1442, to help mark the proximal and distal ends of the stent 1442 or balloon 1440, as well to facilitate alignment of the two catheters during stent deployment, as discussed elsewhere in this specification.

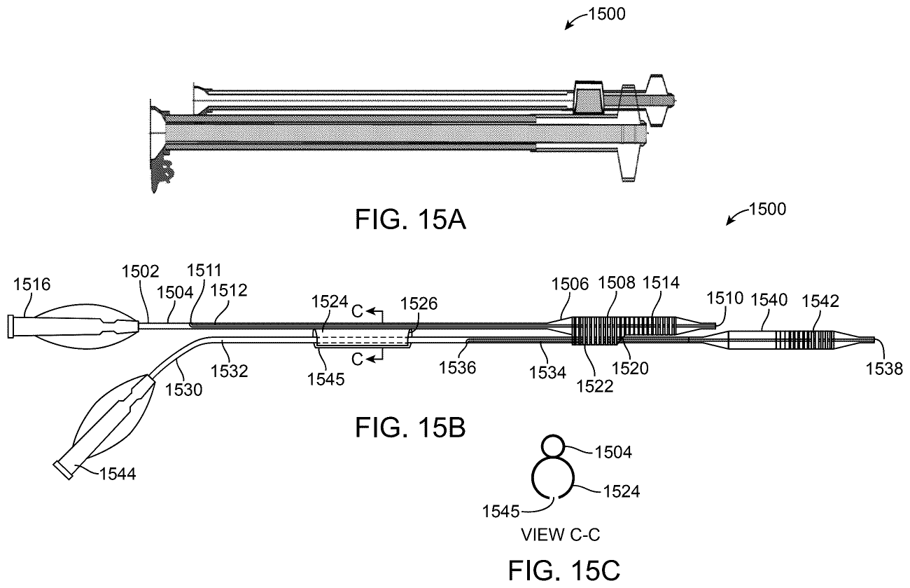

FIG. 15A illustrates a catheter system 1500 having dual rapid exchange design with a short zipper or snap fitting. FIG. 15B more clearly illustrates the features of the catheter system 1500 in FIG. 15A. The catheter system 1500 includes a first catheter 1502 and a second catheter 1530. The first catheter 1502 includes an elongate shaft 1504 with a radially expandable balloon 1506 disposed near a distal end of the elongate shaft 1504. A stent 1508 having a proximal portion 1522, a distal portion 1514 and a side hole 1520 is disposed over the balloon 1506. The distal portion 1514 is crimped to the balloon 1506 to prevent ejection during delivery, while the proximal portion 1522 is partially crimped to the balloon 1506 so the second catheter 1530 may be slidably advanced under the proximal portion 1522 of stent 1508. The first catheter 1502 is a rapid exchange catheter (RX) having a guidewire lumen 1512 extending from the distal guidewire port 1510 at the distal end of the elongate shaft 1504 to a proximal guidewire port 1511, which is closer to the distal guidewire port 1510 than the proximal end of the catheter shaft 1504. A connector 1516 is coupled with the proximal end of the elongate shaft 1504. The connector 1516 is optionally a Luer connector, and this allows easy coupling with an Indeflator or other device for inflation of the balloon 1506. The first catheter 1502 also includes a zipper or snap fitting 1524 coupled to the elongate shaft 1504. The snap fitting 1524 may be coextruded with the first shaft 1504, or it may be bonded or otherwise attached thereto using techniques known to those skilled in the art. The snap fitting 1524 may alternatively be coupled with the other shaft 1532. The snap fitting 1524 includes a central channel 1526 extending therethrough and is sized to slidably receive a portion of the second catheter 1530. An elongate slot 1545 extends along the entire length of the snap fitting 1524 and is sized so that shaft 1532 may snapped into the central channel 1526. FIG. 15C illustrates a partial cross-section of FIG. 15B taken along the line C-C and shows shaft 1504 with the snap fitting 1524. Radiopaque markers may be placed at different locations along the shaft 1504, often near the balloon 1506 and/or stent 1508, to help mark the proximal and distal ends of the stent 1508 or balloon 1506, as well as to facilitate alignment of the two catheters during stent deployment, as discussed elsewhere in this specification.

The second catheter 1530 includes an elongate shaft 1532 with a radially expandable balloon 1540 disposed near a distal end of the elongate shaft 1532. A stent 1542 is disposed over balloon 1540. The stent 1542 may have a length that matches the working length of the balloon 1540, or the stent length may be shorter than the balloon working length. In some examples, the stent 1542 is shorter than the working length of the balloon 1540 so that a proximal portion of the balloon 1540 is unconstrained by the stent 1542, and this unconstrained portion of the balloon 1540 may be slidably advanced or retracted through side hole 1520 and under proximal portion 1522 of stent 1508 as will be discussed below. Stent 1542 is crimped to balloon 1540 to prevent ejection during delivery. At least a portion of balloon 1540 and stent 1542 are distally offset relative to balloon 1506 and stent 1508 so as to minimize profile of the device. In this example the distal stent 1542 may be deployed in a main branch of the vessel and the other stent 1508 may be deployed in a side branch of the vessel. Alternatively, the distal stent 1542 may be deployed in a side branch of a vessel and the other stent 1508 may be deployed in the main branch of a vessel.

The second catheter 1530 is a rapid exchange catheter (RX) having a guidewire lumen 1534 extending from the distal guidewire port 1538 at the distal end of the elongate shaft 1532 to a proximal guidewire port 1536, which is closer to the distal guidewire port 1538 than the proximal end of the catheter shaft 1532. The proximal guidewire port 1536 is also unobstructed by the snap fitting 1524 and may be distal thereto. A connector 1544, optionally a Luer connector, is connected to the proximal end of the elongate shaft 1532 and allows an Indeflator or other device to be coupled with an inflation lumen (not shown) in elongate shaft 1532 for inflation of balloon 1540. A portion of shaft 1532 is snapped into the central channel 1526 of the snap fitting 1524 via slot 1545, and thus shaft 1532 may slide in channel 1526. This helps keep the two catheter shafts 1504, 1532 parallel and prevents tangling during delivery and as shaft 1532 is slidably advanced or retracted relative to shaft 1504. Also, another portion of shaft 1532 is disposed under proximal portion 1522 of stent 1508. The second catheter 1530 may also be slidably advanced or retracted under the proximal portion 1522 of stent 1508 so that the shaft 1532 passes through the side hole 1520 in stent 1508. Radiopaque markers may be placed at different locations on the shaft 1532, often near the balloon 1540 or stent 1542, to help mark the proximal and distal ends of the stent 1542 or balloon 1540, as well as to facilitate alignment of the two catheters during stent deployment, as discussed elsewhere in this specification.

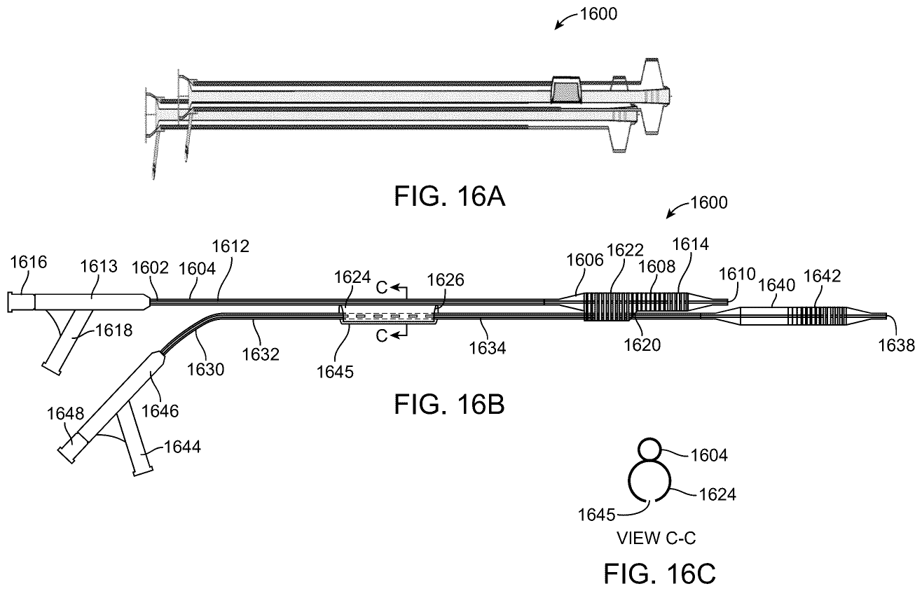

FIG. 16A illustrates a catheter system 1600 having a dual over-the-wire design with a short zipper or snap fitting. FIG. 16B more clearly illustrates the features of the catheter system 1600 in FIG. 16A. The catheter system 1600 includes a first catheter 1602 and a second catheter 1630. The first catheter 1602 includes an elongate shaft 1604 with a radially expandable balloon 1606 disposed near a distal end of the elongate shaft 1604. A stent 1608 having a proximal portion 1622, a distal portion 1614 and a side hole 1620 is disposed over the balloon 1606. The distal portion 1614 is crimped to the balloon 1606 to prevent ejection during delivery, while the proximal portion 1622 is partially crimped to the balloon 1606 so the second catheter 1630 may be slidably advanced under the proximal portion 1622 of stent 1608. The first catheter 1602 is an over-the-wire (OTW) catheter having a guidewire lumen 1612 extending from the distal guidewire port 1610 at the distal end of the elongate shaft 1604 to the proximal end of the elongate shaft 1604 into Y-adapter 1613 having a connector 1616. The connector 1616 is optionally a Luer connector, and this allows easy coupling with a syringe or other device for lumen flushing or injecting contrast media. When unconnected, the guidewire lumen 1612 exits via connector 1616. A second connector 1618, also optionally a Luer connector, allows attachment of an Indeflator or other device to the first catheter 1602 for inflation of the balloon 1606 via an inflation lumen (not shown) in the elongate shaft 1604. The first catheter 1602 also includes a zipper or snap fitting 1624 coupled to the elongate shaft 1604. The snap fitting 1624 may be coextruded with the first shaft 1604, or it may be bonded or otherwise attached thereto using techniques known to those skilled in the art. The snap fitting 1624 may alternatively be coupled with the other shaft 1632. The snap fitting 1624 includes a central channel 1626 extending therethrough and is sized to slidably receive a portion of the second catheter 1630. An elongate slot 1645 extends along the entire length of the snap fitting 1624 and is sized so that shaft 1636 may snapped into the central channel 1626. FIG. 16C illustrates a partial cross-section of FIG. 16B taken along the line C-C and shows shaft 1604 with the snap fitting 1624. Radiopaque markers may be placed at different locations along the shaft 1604, often near the balloon 1606 and/or stent 1608, to help mark the proximal and distal ends of the stent or balloon, as well as to facilitate alignment of the two catheters during stent deployment, as discussed elsewhere in this specification.

The second catheter 1630 includes an elongate shaft 1632 with a radially expandable balloon 1640 disposed near a distal end of the elongate shaft 1632. A stent 1642 is disposed over balloon 1640. The stent 1642 may have a length that matches the working length of the balloon 1640, or the stent length may be shorter than the balloon working length. In some examples, the stent 1642 is shorter than the working length of the balloon 1640 so that a proximal portion of the balloon 1640 is unconstrained by the stent 1642, and this unconstrained portion of the balloon 1640 may be slidably advanced or retracted through side hole 1620 and under proximal portion 1622 of stent 1608 as will be discussed below. Stent 1642 is crimped to balloon 1640 to prevent ejection during delivery. At least a portion of balloon 1640 and stent 1642 are distally offset relative to balloon 1606 and stent 1608 so as to minimize profile of the device. In this example the distal stent 1642 may be deployed in a main branch of the vessel and the other stent 1608 may be deployed in a side branch of the vessel. Alternatively, the distal stent 1642 may be deployed in a side branch of a vessel and the other stent 1608 may be deployed in the main branch of a vessel.

The second catheter 1630 is an over-the-wire (OTW) catheter having a guidewire lumen 1634 extending from the distal guidewire port 1638 at the distal end of the elongate shaft 1632 to the proximal end of the elongate shaft 1632 into Y-adapter 1646 having a connector 1648. The connector 1648 is optionally a Luer connector, and this allows easy coupling with a syringe or other device for lumen flushing or injecting contrast media. When unconnected, the guidewire lumen 1634 exits via connector 1648. A second connector 1644, also optionally a Luer connector, allows attachment of an Indeflator or other device to the second catheter 1630 for inflation of the balloon 1640 via an inflation lumen (not shown) in the elongate shaft 1632. A portion of shaft 1632 is snapped into the central channel 1626 of the snap fitting 1624 via slot 1645, and thus shaft 1632 may slide in channel 1626. This helps keep the two catheter shafts 1604, 1632 parallel and prevents tangling during delivery and as shaft 1632 is slidably advanced or retracted relative to shaft 1604. Also, another portion of shaft 1632 is disposed under proximal portion 1622 of stent 1608. The second catheter 1630 may also be slidably advanced or retracted under the proximal portion 1622 of stent 1608 so that the shaft 1632 passes through the side hole 1620 in stent 1608. Radiopaque markers may be placed at different locations on the shaft 1632, often near the balloon 1640 or stent 1642, to help mark the proximal and distal ends of the stent 1642 or balloon 1640, as well to facilitate alignment of the two catheters during stent deployment, as discussed elsewhere in this specification.

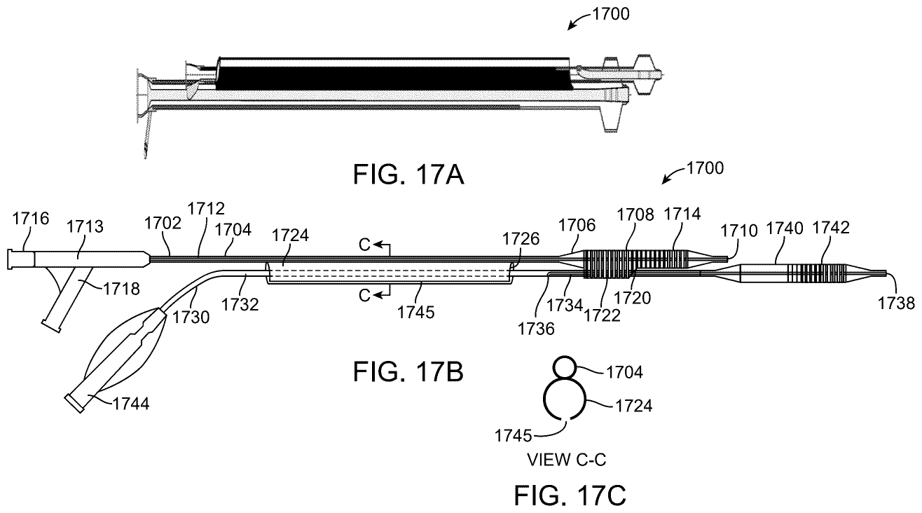

FIG. 17A illustrates a catheter system 1700 having a distal daughter catheter with a rapid-exchange configuration and a proximal mother catheter with an over-the-wire configuration and an end-to-end zipper, or snap fitting. This example is similar to that shown in FIGS. 13A-13B, with the major difference being the length of the snap fitting and the location of one of the guidewire ports.

FIG. 17B more clearly illustrates the features of the catheter system 1700 in FIG. 17A. The catheter system 1700 includes a first catheter 1702 and a second catheter 1730. The first catheter 1702 includes an elongate shaft 1704 with a radially expandable balloon 1706 disposed near a distal end of the elongate shaft 1704. A stent 1708 having a proximal portion 1722, a distal portion 1714 and a side hole 1720 is disposed over the balloon 1706. The distal portion 1714 is crimped to the balloon 1706 to prevent ejection during delivery, while the proximal portion 1722 is partially crimped to the balloon 1706 so the second catheter 1730 may be slidably advanced under the proximal portion 1722 of stent 1708. The first catheter 1702 is an over-the-wire (OTW) catheter having a guidewire lumen 1712 extending from the distal guidewire port 1710 at the distal end of the elongate shaft 1704 to the proximal end of the elongate shaft 1704 into Y-adapter 1713 having a connector 1716. The connector 1716 is optionally a Luer connector, and this allows easy coupling with a syringe or other device for lumen flushing or injecting contrast media. When unconnected, the guidewire lumen 1712 exits via connector 1716. A second connector 1718, also optionally a Luer connector, allows attachment of an Indeflator or other device to the first catheter 1702 for inflation of the balloon 1706 via an inflation lumen (not shown) in the elongate shaft 1704. The first catheter 1702 also includes a zipper or snap fitting 1724 coupled to the elongate shaft 1704. The snap fitting 1724 may be coextruded with the first shaft 1704, or it may be bonded or otherwise attached thereto using techniques known to those skilled in the art. The snap fitting 1724 may alternatively be coupled with the other shaft 1732. The snap fitting 1724 includes a central channel 1726 extending therethrough and is sized to slidably receive a portion of the second catheter 1730. An elongate slot 1745 extends along the entire length of the snap fitting 1724 and is sized so that shaft 1732 may be snapped into the central channel 1726. The snap fitting 1724 may extend from the distal end of connectors 1714, 1744 to the proximal end of balloon 1706, or it may be shorter, extending only partially between the connectors 1714, 1744 and the balloon 1706.

FIG. 17C illustrates a partial cross-section of FIG. 17B taken along the line C-C and shows shaft 1704 with the snap fitting 1724. Radiopaque markers may be placed at different locations along the shaft 1704, often near the balloon 1706 and/or stent 1708, to help mark the proximal and distal ends of the stent 1708 or balloon 1706, as well to facilitate alignment of the two catheters during stent deployment, as discussed elsewhere in this specification.

The second catheter 1730 includes an elongate shaft 1732 with a radially expandable balloon 1740 disposed near a distal end of the elongate shaft 1732. A stent 1742 is disposed over balloon 1740. The stent 1742 may have a length that matches the working length of the balloon 1740, or the stent length may be shorter than the balloon working length. In some examples, the stent 1742 is shorter than the working length of the balloon 1740 so that a proximal portion of the balloon 1740 is unconstrained by the stent 1742, and this unconstrained portion of the balloon 1740 may be slidably advanced or retracted through side hole 1720 and under proximal portion 1722 of stent 1708 as will be discussed below. Stent 1742 is crimped to balloon 1740 to prevent ejection during delivery. At least a portion of balloon 1740 and stent 1742 are distally offset relative to balloon 1706 and stent 1708 so as to minimize profile of the device. In this example the distal stent 1742 may be deployed in a main branch of the vessel and the other stent 1708 may be deployed in a side branch of the vessel. Alternatively, the distal stent 1742 may be deployed in a side branch of a vessel and the other stent 1708 may be deployed in the main branch of a vessel.

The second catheter 1730 is a rapid-exchange catheter (RX) having a guidewire lumen 1734 extending from the distal guidewire port 1738 at the distal end of the elongate shaft 1732 to a proximal guidewire port 1736, which is closer to the distal guidewire port 1738 than the proximal end of the catheter shaft 1732. The proximal guidewire port 1736 is also unobstructed by the snap fitting 1724 and optionally distal thereto. A connector 1744, optionally a Luer connector, is connected to the proximal end of the elongate shaft 1732 and allows an Indeflator or other device to be coupled with an inflation lumen (not shown) in elongate shaft 1732 for inflation of balloon 1740. A portion of shaft 1732 is snapped into the central channel 1726 of the snap fitting 1724 via slot 1745, and thus shaft 1732 may slide in channel 1726. This helps keep the two catheter shafts 1704, 1732 parallel and prevents tangling during delivery and as shaft 1732 is slidably advanced or retracted relative to shaft 1704. Also, another portion of shaft 1732 is disposed under proximal portion 1722 of stent 1708. The second catheter 1730 may also be slidably advanced or retracted under the proximal portion 1722 of stent 1708 so that the shaft 1732 passes through the side hole 1720 in stent 1708. Radiopaque markers may be placed at different locations on the shaft 1732, often near the balloon 1740 or stent 1742, to help mark the proximal and distal ends of the stent 1742 or balloon 1740, as well to facilitate alignment of the two catheters during stent deployment, as discussed elsewhere in this specification.

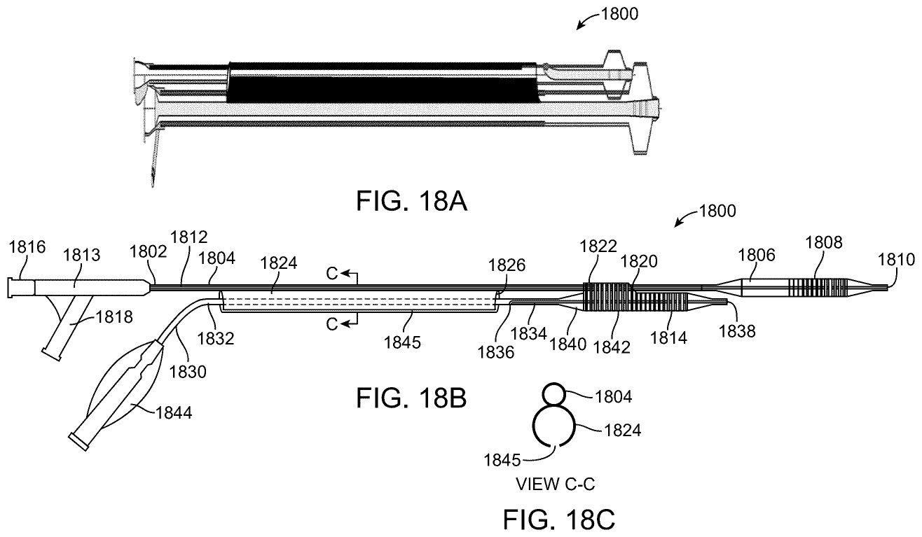

FIG. 18A illustrates a catheter system 1800 having a proximal mother catheter with a rapid-exchange configuration and a distal daughter catheter with an end-to-end zipper or snap fitting. FIG. 18A is similar to the example of FIGS. 14A-14B, with the major difference being the length of the snap fitting and the location of one of the guidewire ports.

FIG. 18B more clearly illustrates the features of the catheter system 1800 in FIG. 18A. The catheter system 1800 includes a first catheter 1802 and a second catheter 1830. The first catheter 1802 includes an elongate shaft 1804 with a radially expandable balloon 1806 disposed near a distal end of the elongate shaft 1804, and a stent 1808 disposed over the balloon 1806. The stent 1808 may be the same length as the working length of the balloon 1806, or it may be shorter. In some examples, the stent 1808 is shorter than the working length of balloon 1806 such that a proximal portion of balloon 1806 remains unconstrained by stent 1808. The proximal portion of balloon 1806 may be slidably advanced and retracted under stent 1842 via side hole 1820. Stent 1808 is crimped to the balloon 1806 to prevent ejection during delivery. The first catheter 1802 is an over-the-wire (OTW) catheter having a guidewire lumen 1812 extending from the distal guidewire port 1810 at the distal end of the elongate shaft 1804 to the proximal end of the elongate shaft 1804 into Y-adapter 1813 having a connector 1816. The connector 1816 is optionally a Luer connector, and this allows easy coupling with a syringe or other device for lumen flushing or injecting contrast media. When unconnected, the guidewire lumen 1812 exits via connector 1816. A second connector 1818, also optionally a Luer connector, allows attachment of an Indeflator or other device to the first catheter 1802 for inflation of the balloon 1806 via an inflation lumen (not shown) in the elongate shaft 1804. The first catheter 1802 also includes a zipper or snap fitting 1824 coupled to the elongate shaft 1804. The snap fitting 1824 may be coextruded with the first shaft 1804, or it may be bonded or otherwise attached thereto using techniques known to those skilled in the art. The snap fitting 1824 may alternatively be coupled with the other shaft 1832. The snap fitting 1824 includes a central channel 1826 extending therethrough and is sized to slidably receive a portion of the second catheter 1830. An elongate slot 1845 extends along the entire length of the snap fitting 1824 and is sized so that shaft 1832 may be snapped into the central channel 1826. FIG. 18C illustrates a partial cross-section of FIG. 18B taken along the line C-C and shows shaft 1804 with the snap fitting 1824. The snap fitting 1824 may extend from the distal end of connectors 1816, 1844 to the proximal end of balloon 1840, or it may be shorter, extending only partially between the connectors 1816, 1844 and the balloon 1806. Radiopaque markers may be placed at different locations along the shaft 1804, often near the balloon 1806 and/or stent 1808, to help mark the proximal and distal ends of the stent 1808 or balloon 1806, as well as to facilitate alignment of the two catheters during stent deployment, as discussed elsewhere in this specification.

The second catheter 1830 includes an elongate shaft 1832 with a radially expandable balloon 1840 disposed near a distal end of the elongate shaft 1832. A stent 1842 having a proximal portion 1822, a distal portion 1814, and a side hole 1820 is disposed over balloon 1840. The distal portion 1814 is crimped to balloon 1840 to prevent ejection during delivery, while the proximal portion 1822 is partially crimped to balloon 1840 so elongate shaft 1804 may be slidably advanced or retracted under the proximal portion 1822 of stent 1842. The stent 1842 may optionally have a length that matches the working length of the balloon 1840, or the stent length may be shorter than the balloon working length. At least a portion of balloon 1806 and stent 1808 are distally offset relative to balloon 1840 and stent 1842 so as to minimize profile of the device. In this example the distal stent 1808 may be deployed in a main branch of the vessel and the other stent 1842 may be deployed in a side branch of the vessel. Alternatively, the distal stent 1808 may be deployed in a side branch of a vessel and the other stent 1842 may be deployed in the main branch of a vessel. The second catheter 1830 is a rapid exchange catheter (RX) having a guidewire lumen 1834 extending from the distal guidewire port 1838 at the distal end of the elongate shaft 1832 to a proximal guidewire port 1836, which is closer to the distal guidewire port 1838 than the proximal end of the catheter shaft 1832. The proximal guidewire port 1836 is also unobstructed by the snap fitting 1824 and optionally distal thereto. A connector 1844, optionally a Luer connector, is connected to the proximal end of the elongate shaft 1832 and allows an Indeflator or other device to be coupled with an inflation lumen (not shown) in elongate shaft 1832 for inflation of balloon 1840. A portion of shaft 1832 is snapped into the central channel 1826 of the snap fitting 1824 via slot 1845, and thus shaft 1832 may slide in channel 1826. This helps keep the two catheter shafts 1804, 1832 parallel and prevents tangling during delivery and as shaft 1832 is slidably advanced or retracted relative to shaft 1804. Also, a portion of shaft 1804 is disposed under proximal portion 1822 of stent 1842. The first catheter 1802 may be slidably advanced or retracted under the proximal portion 1822 of stent 1842 so that the shaft 1804 passes through the side hole 1820 in stent 1842. Radiopaque markers may be placed at different locations on the shaft 1832, often near the balloon 1840 or stent 1842, to help mark the proximal and distal ends of the stent 1842 or balloon 1840, as well as to facilitate alignment of the two catheters during stent deployment, as discussed elsewhere in this specification.

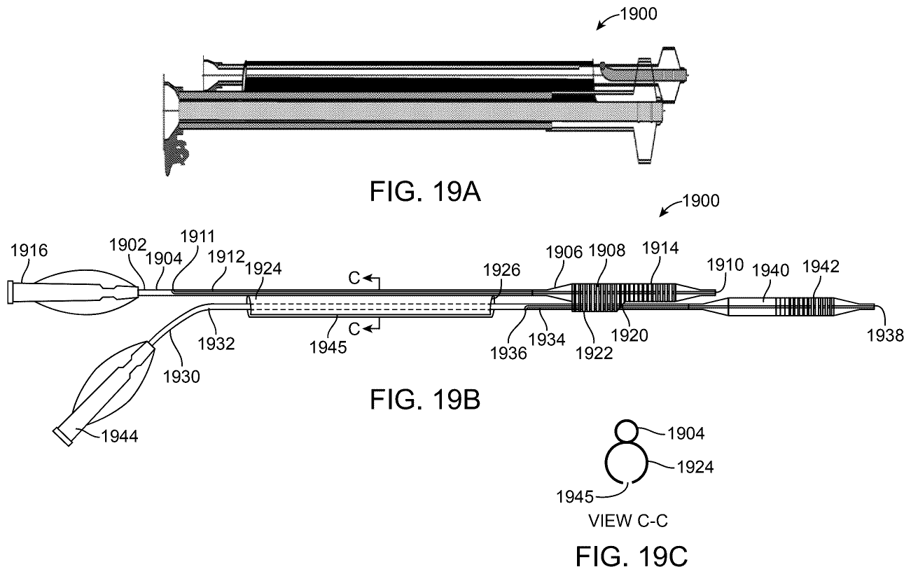

FIG. 19A illustrates a catheter system 1900 having a dual rapid-exchange design with an end-to-end zipper or snap fitting. FIG. 19A is similar to the example of FIGS. 15A-15B, with the major difference being the length of the snap fitting.

FIG. 19B more clearly illustrates the features of the catheter system 1900 in FIG. 19A. The catheter system 1900 includes a first catheter 1902 and a second catheter 1930. The first catheter 1902 includes an elongate shaft 1904 with a radially expandable balloon 1906 disposed near a distal end of the elongate shaft 1904. A stent 1908 having a proximal portion 1922, a distal portion 1914 and a side hole 1920 is disposed over the balloon 1906. The distal portion 1914 is crimped to the balloon 1906 to prevent ejection during delivery, while the proximal portion 1922 is partially crimped to the balloon 1906 so the second catheter 1930 may be slidably advanced under the proximal portion 1922 of stent 1908. The first catheter 1902 is a rapid exchange catheter (RX) having a guidewire lumen 1912 extending from the distal guidewire port 1910 at the distal end of the elongate shaft 1904 to a proximal guidewire port 1911, which is closer to the distal guidewire port 1910 than the proximal end of the catheter shaft 1904. A connector 1916 is coupled with the proximal end of the elongate shaft 1904. The connector 1916 is optionally a Luer connector, and this allows easy coupling with an Indeflator or other device for inflation of the balloon 1906. The first catheter 1902 also includes a zipper or snap fitting 1924 coupled to the elongate shaft 1904. The snap fitting 1924 may be coextruded with the first shaft 1904, or it may be bonded or otherwise attached thereto using techniques known to those skilled in the art. The snap fitting 1924 may alternatively be coupled with the other shaft 1932. The snap fitting 1924 includes a central channel 1926 extending therethrough and is sized to slidably receive a portion of the second catheter 1930. An elongate slot 1945 extends along the entire length of the snap fitting 1924 and is sized so that shaft 1932 may snapped into the central channel 1926. FIG. 19C illustrates a partial cross-section of FIG. 19B taken along the line C-C and shows shaft 1904 with the snap fitting 1924. Radiopaque markers may be placed at different locations along the shaft 1904, often near the balloon 1906 and/or stent 1908, to help mark the proximal and distal ends of the stent 1908 or balloon 1906, as well as to facilitate alignment of the two catheters during stent deployment, as discussed elsewhere in this specification.

The second catheter 1930 includes an elongate shaft 1932 with a radially expandable balloon 1940 disposed near a distal end of the elongate shaft 1932. A stent 1942 is disposed over balloon 1940. The stent 1942 may have a length that matches the working length of the balloon 1940, or the stent length may be shorter than the balloon working length. In some examples, the stent 1942 is shorter than the working length of the balloon 1940 so that a proximal portion of the balloon 1940 is unconstrained by the stent 1942 and this unconstrained portion of the balloon 1940 may be slidably advanced or retracted through side hole 1920 and under proximal portion 1922 of stent 1908 as will be discussed below. Stent 1942 is crimped to balloon 1940 to prevent ejection during delivery. At least a portion of balloon 1940 and stent 1942 are distally offset relative to balloon 1906 and stent 1908 so as to minimize profile of the device. In this example the distal stent 1942 may be deployed in a main branch of the vessel and the other stent 1908 may be deployed in a side branch of the vessel. Alternatively, the distal stent 1942 may be deployed in a side branch of a vessel and the other stent 1908 may be deployed in the main branch of a vessel.

The second catheter 1930 is a rapid exchange catheter (RX) having a guidewire lumen 1934 extending from the distal guidewire port 1938 at the distal end of the elongate shaft 1932 to a proximal guidewire port 1936, which is closer to the distal guidewire port 1938 than the proximal end of the catheter shaft 1932. The proximal guidewire port 1936 is also unobstructed by the snap fitting 1924 and may be distal thereto. A connector 1944, optionally a Luer connector, is connected to the proximal end of the elongate shaft 1932 and allows an Indeflator or other device to be coupled with an inflation lumen (not shown) in elongate shaft 1932 for inflation of balloon 1940. A portion of shaft 1932 is snapped into the central channel 1926 of the snap fitting 1924 via slot 1945, and thus shaft 1932 may slide in channel 1926. This helps keep the two catheter shafts 1904, 1932 parallel and prevents tangling during delivery and as shaft 1932 is slidably advanced or retracted relative to shaft 1904. Also, another portion of shaft 1932 is disposed under proximal portion 1922 of stent 1908. The second catheter 1930 may also be slidably advanced or retracted under the proximal portion 1922 of stent 1908 so that the shaft 1932 passes through the side hole 1920 in stent 1908. Radiopaque markers may be placed at different locations on the shaft 1932, often near the balloon 1940 or stent 1942, to help mark the proximal and distal ends of the stent 1942 or balloon 1940, as well as to facilitate alignment of the two catheters during stent deployment, as discussed elsewhere in this specification.

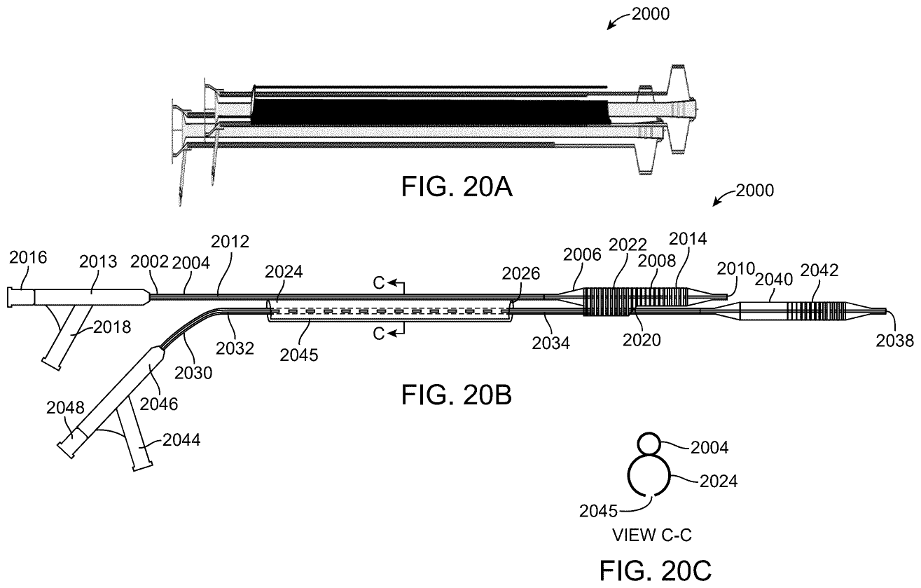

FIG. 20A illustrates a catheter system 2000 having a dual over-the-wire design with an end-to-end zipper or snap fitting. FIG. 20A is similar to the example of FIGS. 16A-16B, with the major difference being the length of the snap fitting.

FIG. 20B more clearly illustrates the features of the catheter system 2000 in FIG. 20A. The catheter system 2000 includes a first catheter 2002 and a second catheter 2030. The first catheter 2002 includes an elongate shaft 2004 with a radially expandable balloon 2006 disposed near a distal end of the elongate shaft 2004. A stent 2008 having a proximal portion 2022, a distal portion 2014 and a side hole 2020 is disposed over the balloon 2006. The distal portion 2014 is crimped to the balloon 2006 to prevent ejection during delivery, while the proximal portion 2022 is partially crimped to the balloon 2006 so the second catheter 2030 may be slidably advanced under the proximal portion 2022 of stent 2008. The first catheter 2002 is an over-the-wire (OTW) catheter having a guidewire lumen 2012 extending from the distal guidewire port 2010 at the distal end of the elongate shaft 2004 to the proximal end of the elongate shaft 2004 into Y-adapter 2013 having a connector 2016. The connector 2016 is optionally a Luer connector, and this allows easy coupling with a syringe or other device for lumen flushing or injecting contrast media. When unconnected, the guidewire lumen 2012 exits via connector 2016. A second connector 2018, also optionally a Luer connector, allows attachment of an Indeflator or other device to the first catheter 2002 for inflation of the balloon 2006 via an inflation lumen (not shown) in the elongate shaft 2004. The first catheter 2002 also includes a zipper or snap fitting 2024 coupled to the elongate shaft 2004. The snap fitting 2024 may be coextruded with the first shaft 2004, or it may be bonded or otherwise attached thereto using techniques known to those skilled in the art. The snap fitting 2024 may alternatively be coupled with the other shaft 2032. The snap fitting 2024 includes a central channel 2026 extending therethrough and is sized to slidably receive a portion of the second catheter 2030. An elongate slot 2045 extends along the entire length of the snap fitting 2024 and is sized so that shaft 2036 may snapped into the central channel 2026. FIG. 20C illustrates a partial cross-section of FIG. 20B taken along the line C-C and shows shaft 2004 with the snap fitting 2024. Radiopaque markers may be placed at different locations along the shaft 2004, often near the balloon 2006 and/or stent 2008, to help mark the proximal and distal ends of the stent 2008 or balloon 2006, as well as to facilitate alignment of the two catheters during stent deployment, as discussed elsewhere in this specification.

The second catheter 2030 includes an elongate shaft 2032 with a radially expandable balloon 2040 disposed near a distal end of the elongate shaft 2032. A stent 2042 is disposed over balloon 2040. The stent 2042 may have a length that matches the working length of the balloon 2040, or the stent length may be shorter than the balloon working length. In some examples, the stent 2042 is shorter than the working length of the balloon 2040 so that a proximal portion of the balloon 2040 is unconstrained by the stent 2042, and this unconstrained portion of the balloon 2040 may be slidably advanced or retracted through side hole 2020 and under proximal portion 2022 of stent 2008 as will be discussed below. Stent 2042 is crimped to balloon 2040 to prevent ejection during delivery. At least a portion of balloon 2040 and stent 2042 are distally offset relative to balloon 2006 and stent 2008 so as to minimize profile of the device. In this example the distal stent 2042 may be deployed in a main branch of the vessel and the other stent 2008 may be deployed in a side branch of the vessel. Alternatively, the distal stent 2042 may be deployed in a side branch of a vessel and the other stent 2008 may be deployed in the main branch of a vessel.

The second catheter 2030 is an over-the-wire (OTW) catheter having a guidewire lumen 2034 extending from the distal guidewire port 2038 at the distal end of the elongate shaft 2032 to the proximal end of the elongate shaft 2032 into Y-adapter 2046 having a connector 2048. The connector 2048 is optionally a Luer connector, and this allows easy coupling with a syringe or other device for lumen flushing or injecting contrast media. When unconnected, the guidewire lumen 2034 exits via connector 2048. A second connector 2044, also optionally a Luer connector, allows attachment of an Indeflator or other device to the second catheter 2030 for inflation of the balloon 2040 via an inflation lumen (not shown) in the elongate shaft 2032. A portion of shaft 2032 is snapped into the central channel 2026 of the snap fitting 2024 via slot 2045, and thus shaft 2032 may slide in channel 2026. This helps keep the two catheter shafts 2004, 2032 parallel and prevents tangling during delivery and as shaft 2032 is slidably advanced or retracted relative to shaft 2004. Also, another portion of shaft 2032 is disposed under proximal portion 2022 of stent 2008. The second catheter 2030 may also be slidably advanced or retracted under the proximal portion 2022 of stent 2008 so that the shaft 2032 passes through the side hole 2020 in stent 2008. Radiopaque markers may be placed at different locations on the shaft 2032, often near the balloon 2040 or stent 2042, to help mark the proximal and distal ends of the stent 2042 or balloon 2040, as well to facilitate alignment of the two catheters during stent deployment, as discussed elsewhere in this specification.

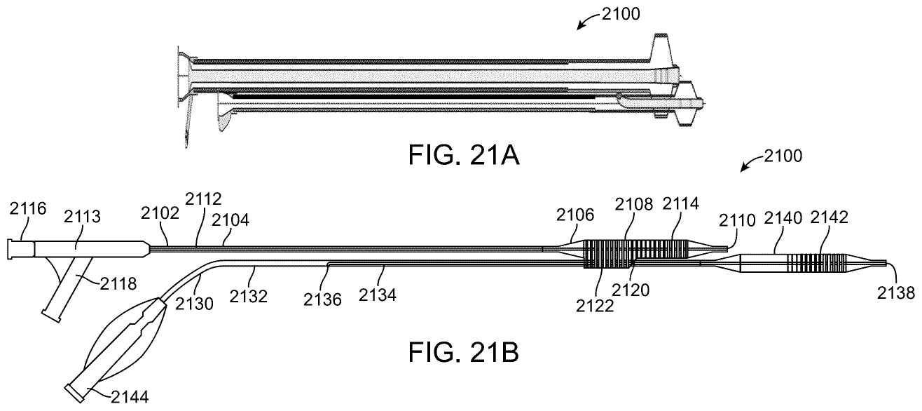

FIGS. 21A, 22A, 23A, and 24A illustrate catheters that can be used with an alternative example where the mother catheter is provided to the operator with a mother stent that is crimped on the distal portion of the mother catheter balloon. The proximal portion of the mother stent is uncrimped or partially crimped. The operator can mount any commercially available catheter or balloon on a wire through the mother stent proximal end and exit out the side hole of the mother stent. The operator can align the catheters to suit the patient's anatomy and crimp the proximal portion of the mother stent. The operator can crimp the stent tightly so that the catheters do not move relative to each other. It is possible for the operator to place the catheters at the bifurcation and, if necessary, pull back on the commercially available catheter to adjust the alignment. Then the operator can gently push the system distally to ensure complete apposition.

FIG. 21A illustrates a catheter system 2100 having a distal daughter catheter with a rapid exchange configuration and a proximal mother catheter with an over-the-wire configuration.

FIG. 21B more clearly illustrates the features of the catheter system 2100 in FIG. 21A. The catheter system 2100 includes a first catheter 2102 and a second catheter 2130. The first catheter 2102 includes an elongate shaft 2104 with a radially expandable balloon 2106 disposed near a distal end of the elongate shaft 2104. A stent 2108 having a proximal portion 2122, a distal portion 2114 and a side hole 2120 is disposed over the balloon 2106. The distal portion 2114 is crimped to the balloon 2106 to prevent ejection during delivery, while the proximal portion 2122 is partially crimped to the balloon 2106 so the second catheter 2130 may be slidably advanced under the proximal portion 2122 of stent 2108. The first catheter 2102 is an over-the-wire (OTW) catheter having a guidewire lumen 2112 extending from the distal guidewire port 2110 at the distal end of the elongate shaft 2104 to the proximal end of the elongate shaft 2104 into Y-adapter 2113 having a connector 2116. The connector 2116 is optionally a Luer connector, and this allows easy coupling with a syringe or other device for lumen flushing or injecting contrast media. When unconnected, the guidewire lumen 2112 exits via connector 2116. A second connector 2118, also optionally a Luer connector, allows attachment of an Indeflator or other device to the catheter for inflation of the balloon 2106 via an inflation lumen (not shown) in the elongate shaft 2104. Radiopaque markers may be placed at different locations along the shaft 2104, often near the balloon 2106 and/or stent 2108, to help mark the proximal and distal ends of the stent 2108 or balloon 2106, as well as to facilitate alignment of the two catheters during stent deployment, as discussed elsewhere in this specification.

The second catheter 2130 includes an elongate shaft 2132 with a radially expandable balloon 2140 disposed near a distal end of the elongate shaft 2132. A stent 2142 is disposed over balloon 2140. The stent 2142 may have a length that matches the working length of the balloon 2140, or the stent length may be shorter than the balloon working length. In some examples, the stent 2142 is shorter than the working length of the balloon 2140 so that a proximal portion of the balloon 2140 is unconstrained by the stent 2142, and this unconstrained portion of the balloon 2140 may be slidably advanced or retracted through side hole 2120 and under proximal portion 2122 of stent 2108 as will be discussed below. Stent 2142 is crimped to balloon 2140 to prevent ejection during delivery. At least a portion of balloon 2140 and stent 2142 are distally offset relative to balloon 2106 and stent 2108 so as to minimize profile of the device. In this example the distal stent 2142 may be deployed in a main branch of the vessel and the other stent 2108 may be deployed in a side branch of the vessel. Alternatively, the distal stent 2142 may be deployed in a side branch of a vessel and the other stent 2108 may be deployed in the main branch of a vessel.

The second catheter 2130 is a rapid exchange catheter (RX) having a guidewire lumen 2134 extending from the distal guidewire port 2138 at the distal end of the elongate shaft 2132 to a proximal guidewire port 2136, which is closer to the distal guidewire port 2138 than the proximal end of the catheter shaft 2132. A connector 2144, optionally a Luer connector, is connected to the proximal end of the elongate shaft 2132 and allows an Indeflator or other device to be coupled with an inflation lumen (not shown) in elongate shaft 2132 for inflation of balloon 2140. Having a portion of shaft 2132 disposed under proximal portion 2122 of stent 2108 helps keep catheter shafts 2104, 2132 parallel and prevents tangling during delivery and as shaft 2132 is slidably advanced or retracted relative to shaft 2104. Also, another portion of shaft 2132 is disposed under proximal portion 2122 of stent 2108. The second catheter 2130 may also be slidably advanced or retracted under the proximal portion 2122 of stent 2108 so that the shaft 2132 passes through the side hole 2120 in stent 2108. Radiopaque markers may be placed at different locations on the shaft 2132, often near the balloon 2140 or stent 2142, to help mark the proximal and distal ends of the stent 2142 or balloon 2140, as well as to facilitate alignment of the two catheters during stent deployment, as discussed elsewhere in this specification.

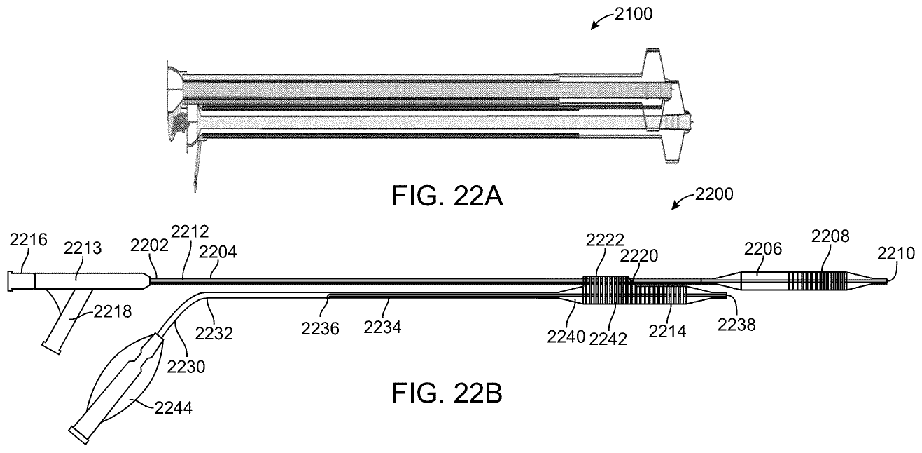

FIG. 22A illustrates a catheter system 2200 having a proximal mother catheter with an over-the-wire design and a distal daughter catheter with an over-the-wire configuration.

FIG. 22B more clearly illustrates the features of the catheter system 2200 in FIG. 22A. The catheter system 2200 includes a first catheter 2202 and a second catheter 2230. The first catheter 2202 includes an elongate shaft 2204 with a radially expandable balloon 2206 disposed near a distal end of the elongate shaft 2204, and a stent 2208 disposed over the balloon 2206. The stent 2208 may be the same length as the working length of the balloon 2206, or it may be shorter. In some examples, the stent 2208 is shorter than the working length of balloon 2206 such that a proximal portion of balloon 2206 remains unconstrained by stent 2208. The proximal portion of balloon 2206 may be slidably advanced and retracted under stent 2242 via side hole 2220. Stent 2208 is crimped to the balloon 2206 to prevent ejection during delivery. The first catheter 2202 is an over-the-wire (OTW) catheter having a guidewire lumen 2212 extending from the distal guidewire port 2210 at the distal end of the elongate shaft 2204 to the proximal end of the elongate shaft 2204 into Y-adapter 2213 having a connector 2216. The connector 2216 is optionally a Luer connector, and this allows easy coupling with a syringe or other device for lumen flushing or injecting contrast media. When unconnected, the guidewire lumen 2212 exits via connector 2216. A second connector 2218, also optionally a Luer connector, allows attachment of an Indeflator or other device to the catheter for inflation of the balloon 2206 via an inflation lumen (not shown) in the elongate shaft 2204. Radiopaque markers may be placed at different locations along the shaft 2204, often near the balloon 2206 and/or stent 2208, to help mark the proximal and distal ends of the stent 2208 or balloon 2206, as well as to facilitate alignment of the two catheters during stent deployment, as discussed elsewhere in this specification.

The second catheter 2230 includes an elongate shaft 2232 with a radially expandable balloon 2240 disposed near a distal end of the elongate shaft 2232. A stent 2242 having a proximal portion 2222, a distal portion 2214, and a side hole 2220 is disposed over balloon 2240. The distal portion 2214 is crimped to balloon 2240 to prevent ejection during delivery, while the proximal portion 2222 is partially crimped to balloon 2240 so elongate shaft 2204 may be slidably advanced or retracted under the proximal portion 2222 of stent 2242. The stent 2242 may optionally have a length that matches the working length of the balloon 2240, or the stent length may be shorter than the balloon working length. At least a portion of balloon 2206, and stent 2208 are distally offset relative to balloon 2240 and stent 2242 so as to minimize profile of the device. In this example the distal stent 2208 may be deployed in a main branch of the vessel and the other stent 2242 may be deployed in a side branch of the vessel. Alternatively, the distal stent 2208 may be deployed in a side branch of a vessel and the other stent 2242 may be deployed in the main branch of a vessel.

The second catheter 2230 is a rapid-exchange catheter (RX) having a guidewire lumen 2234 extending from the distal guidewire port 2238 at the distal end of the elongate shaft 2232 to a proximal guidewire port 2236, which is closer to the distal guidewire port 2238 than the proximal end of the catheter shaft 2232. A connector 2244, optionally a Luer connector, is connected to the proximal end of the elongate shaft 2232 and allows an Indeflator or other device to be coupled with an inflation lumen (not shown) in elongate shaft 2232 for inflation of balloon 2240. Having a portion of shaft 2204 disposed under proximal portion 2222 of stent 2208 helps keep catheters 2202, 2232 parallel and prevents tangling during delivery and as shaft 2204 is slidably advanced or retracted relative to shaft 2232. The first catheter 2202 may be slidably advanced or retracted under the proximal portion 2222 of stent 2242 so that the shaft 2204 passes through the side hole 2220 in stent 2242. Radiopaque markers may be placed at different locations on the shaft 2232, often near the balloon 2240 or stent 2242, to help mark the proximal and distal ends of the stent 2242 or balloon 2240, as well to facilitate alignment of the two catheters during stent deployment, as discussed elsewhere in this specification.

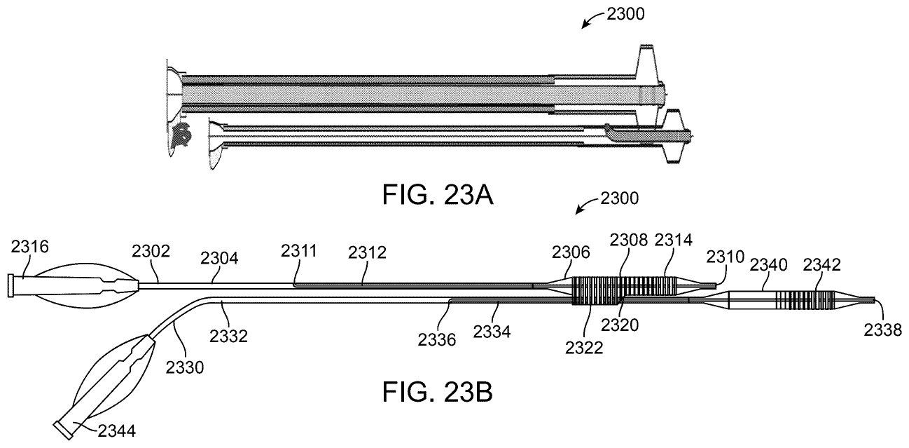

FIG. 23A illustrates a catheter system 2300 having a dual rapid exchange design. FIG. 23B more clearly illustrates the features of the catheter system 2300 in FIG. 23A. The catheter system 2300 includes a first catheter 2302 and a second catheter 2330. The first catheter 2302 includes an elongate shaft 2304 with a radially expandable balloon 2306 disposed near a distal end of the elongate shaft 2304. A stent 2308 having a proximal portion 2322, a distal portion 2314 and a side hole 2320 is disposed over the balloon 2306. The distal portion 2314 is crimped to the balloon 2306 to prevent ejection during delivery, while the proximal portion 2322 is partially crimped to the balloon 2306 so the second catheter 2330 may be slidably advanced under the proximal portion 2322 of stent 2308. The first catheter 2302 is a rapid exchange catheter (RX) having a guidewire lumen 2312 extending from the distal guidewire port 2310 at the distal end of the elongate shaft 2304 to a proximal guidewire port 2311, which is closer to the distal guidewire port 2310 than the proximal end of the catheter shaft 2304. A connector 2316 is coupled with the proximal end of the elongate shaft 2304. The connector 2116 is optionally a Luer connector, and this allows easy coupling with an Indeflator or other device for inflation of the balloon 2306. Radiopaque markers may be placed at different locations along the shaft 2304, often near the balloon 2306 and/or stent 2308, to help mark the proximal and distal ends of the stent 2308 or balloon 2306, as well as to facilitate alignment of the two catheters during stent deployment, as discussed elsewhere in this specification.

The second catheter 2330 includes an elongate shaft 2332 with a radially expandable balloon 2340 disposed near a distal end of the elongate shaft 2332. A stent 2342 is disposed over balloon 2340. The stent 2342 may have a length that matches the working length of the balloon 2340, or the stent length may be shorter than the balloon working length. In some examples, the stent 2342 is shorter than the working length of the balloon 2340 so that a proximal portion of the balloon 2340 is unconstrained by the stent 2342, and this unconstrained portion of the balloon 2340 may be slidably advanced or retracted through side hole 2320 and under proximal portion 2322 of stent 2308 as will be discussed below. Stent 2342 is crimped to balloon 2340 to prevent ejection during delivery. At least a portion of balloon 2340 and stent 2342 are distally offset relative to balloon 2306 and stent 2308 so as to minimize profile of the device. In this example the distal stent 2342 may be deployed in a main branch of the vessel and the other stent 2308 may be deployed in a side branch of the vessel. Alternatively, the distal stent 2342 may be deployed in a side branch of a vessel and the other stent 2308 may be deployed in the main branch of a vessel.

The second catheter 2330 is a rapid exchange catheter (RX) having a guidewire lumen 2334 extending from the distal guidewire port 2338 at the distal end of the elongate shaft 2332 to a proximal guidewire port 2336, which is closer to the distal guidewire port 2338 than the proximal end of the catheter shaft 2332. A connector 2344, optionally a Luer connector, is connected to the proximal end of the elongate shaft 2332 and allows an Indeflator or other device to be coupled with an inflation lumen (not shown) in elongate shaft 2332 for inflation of balloon 2340. Having a portion of shaft 2332 disposed under proximal portion 2322 of stent 2208 helps keep catheters 2302, 2330 parallel and prevents tangling during delivery and as shaft 2332 is slidably advanced or retracted relative to shaft 2304. The second catheter 2330 may also be slidably advanced or retracted under the proximal portion 2322 of stent 2308 so that the shaft 2332 passes through the side hole 2320 in stent 2308. Radiopaque markers may be placed at different locations on the shaft 2332, often near the balloon 2340 or stent 2342, to help mark the proximal and distal ends of the stent 2342 or balloon 2340, as well as to facilitate alignment of the two catheters during stent deployment, as discussed elsewhere in this specification.

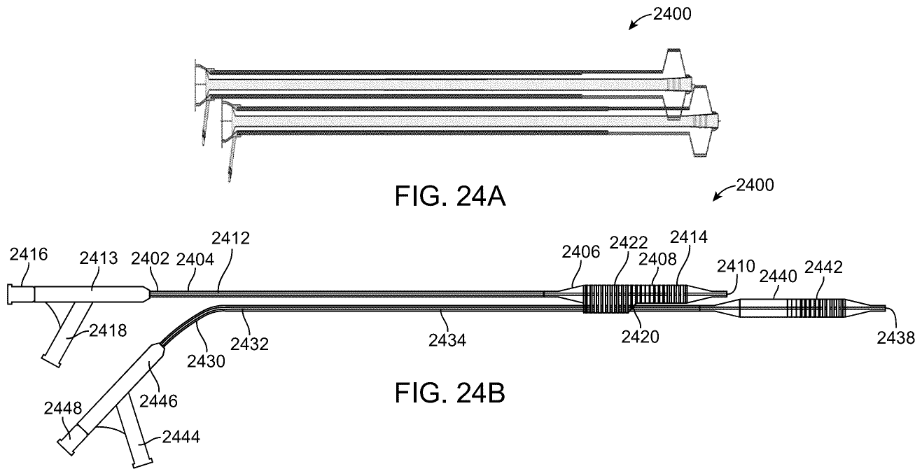

FIG. 24A illustrates a catheter system 2400 having a dual over-the-wire design. FIG. 24B more clearly illustrates the features of the catheter system 2400 in FIG. 24A. The catheter system 2400 includes a first catheter 2402 and a second catheter 2430. The first catheter 2402 includes an elongate shaft 2404 with a radially expandable balloon 2406 disposed near a distal end of the elongate shaft 2404. A stent 2408 having a proximal portion 2422, a distal portion 2414 and a side hole 2420 is disposed over the balloon 2406. The distal portion 2414 is crimped to the balloon 2406 to prevent ejection during delivery, while the proximal portion 2422 is partially crimped to the balloon 2406 so the second catheter 2430 may be slidably advanced under the proximal portion 2422 of stent 2408. The first catheter 2402 is an over-the-wire (OTW) catheter having a guidewire lumen 2412 extending from the distal guidewire port 2410 at the distal end of the elongate shaft 2404 to the proximal end of the elongate shaft 2404 into Y-adapter 2413 having a connector 2416. The connector 2416 is optionally a Luer connector, and this allows easy coupling with a syringe or other device for lumen flushing or injecting contrast media. When unconnected, the guidewire lumen 2412 exits via connector 2416. A second connector 2418, also optionally a Luer connector, allows attachment of an Indeflator or other device to the catheter for inflation of the balloon 2406 via an inflation lumen (not shown) in the elongate shaft 2404. Radiopaque markers may be placed at different locations along the shaft 2404, often near the balloon 2406 and/or stent 2408, to help mark the proximal and distal ends of the stent 2408 or balloon 2406, as well as to facilitate alignment of the two catheters during stent deployment, as discussed elsewhere in this specification.

The second catheter 2430 includes an elongate shaft 2432 with a radially expandable balloon 2440 disposed near a distal end of the elongate shaft 2432. A stent 2442 is disposed over balloon 2440. The stent 2442 may have a length that matches the working length of the balloon 2440, or the stent length may be shorter than the balloon working length. In some examples, the stent 2442 is shorter than the working length of the balloon 2440 so that a proximal portion of the balloon 2440 is unconstrained by the stent 2442, and this unconstrained portion of the balloon 2440 may be slidably advanced or retracted through side hole 2420 and under proximal portion 2422 of stent 2408 as will be discussed below. Stent 2442 is crimped to balloon 2440 to prevent ejection during delivery. At least a portion of balloon 2440 and stent 2442 are distally offset relative to balloon 2406 and stent 2408 so as to minimize profile of the device. In this example the distal stent 2442 may be deployed in a main branch of the vessel and the other stent 2408 may be deployed in a side branch of the vessel. Alternatively, the distal stent 2442 may be deployed in a side branch of a vessel and the other stent 2408 may be deployed in the main branch of a vessel.

The second catheter 2430 is an over-the-wire (OTW) catheter having a guidewire lumen 2434 extending from the distal guidewire port 2438 at the distal end of the elongate shaft 2432 to the proximal end of the elongate shaft 2432 into Y-adapter 2446 having a connector 2448. The connector 2448 is optionally a Luer connector, and this allows easy coupling with a syringe or other device for lumen flushing or injecting contrast media. When unconnected, the guidewire lumen 2434 exits via connector 2448. A second connector 2444, also optionally a Luer connector, allows attachment of an Indeflator or other device to the catheter for inflation of the balloon 2440 via an inflation lumen (not shown) in the elongate shaft 2432. Having a portion of shaft 2432 disposed under proximal portion 2422 of stent 2408 helps keep catheters 2402, 2430 parallel and prevents tangling during delivery and as shaft 2432 is slidably advanced or retracted relative to shaft 2404. The second catheter 2430 may also be slidably advanced or retracted under the proximal portion 2422 of stent 2408 so that the shaft 2432 passes through the side hole 2420 in stent 2408. Radiopaque markers may be placed at different locations on the shaft 2432, often near the balloon 2440 or stent 2442, to help mark the proximal and distal ends of the stent 2442 or balloon 2440, as well as to facilitate alignment of the two catheters during stent deployment, as discussed elsewhere in this specification.

In any of the examples disclosed herein, commercially available catheters and commercially available stents may be matched up to form the systems illustrated. In still other examples, commercially available catheters that are single-use devices for treating a single vessel may be mated together in various combinations and coupled together with a polymer sleeve. The operator chooses the two catheters for the patient's anatomy then slides a sized polymer sleeve over both catheters from the distal ends. Once the operator has the catheters aligned, the polymer sleeve can be treated with a heat or light source to shrink and bond the two catheters together with friction. The polymer sleeve is made of typical polymers that can act as shrink wrap when treated with a heat or light source. The polymer of the polymer sleeve for example could be manufactured with polyolefin, a chemical used in manufacturing shrink wrap. The polymer sleeve would not crosslink or covalently attach to the catheters; several types of polymers are commercially available and have the requisite properties-thin, strong, not adhesive- and reaction times to their source of ten minutes or less. The polymer sleeves are typically 15 centimeters in length and have various diameters to suit typical catheter diameters 4 French to 20 French. The operator can test that the bond is holding by applying slight pressure prior to the procedure. If the polymer sleeve does not hold tightly, the operator may elect to use a smaller-diameter polymer sleeve or use more than one polymer sleeve by placing the polymer sleeves adjacent to each other. Alternatively, several smaller sleeves from 1 to 10 centimeters in length could be placed over several different portions of the catheters.

In any of the examples discussed herein, a therapeutic agent may be disposed on the stent or balloon and eluted therefrom in a controlled manner into the target treatment area such as a stenotic lesion. Some therapeutic agents may help inhibit restenosis, hyperplasia or have other therapeutic benefits. Some anti-hyperplasia agents include anti-neoplastic drugs, such as paclitaxel, methotrexate, and batimastal; antibiotics such as doxycycline, tetracycline, rapamycin, everolimus, biolimus A9, novolimus, myolimus, zotarolimus, and other analogs and derivatives of rapamycin and actinomycin; immuno-suppressants such as dexamethasone and methyl prednisolone; nitric oxide sources such as nitroprussides; estrogen; estradiols; and the like. Methods for applying the therapeutic agent to the stent or balloon are well known to those skilled in the art, and have been described in the patent and scientific literature. Further disclosure related to the use of a therapeutic agent may be found elsewhere in this specification.

### Stent Delivery:

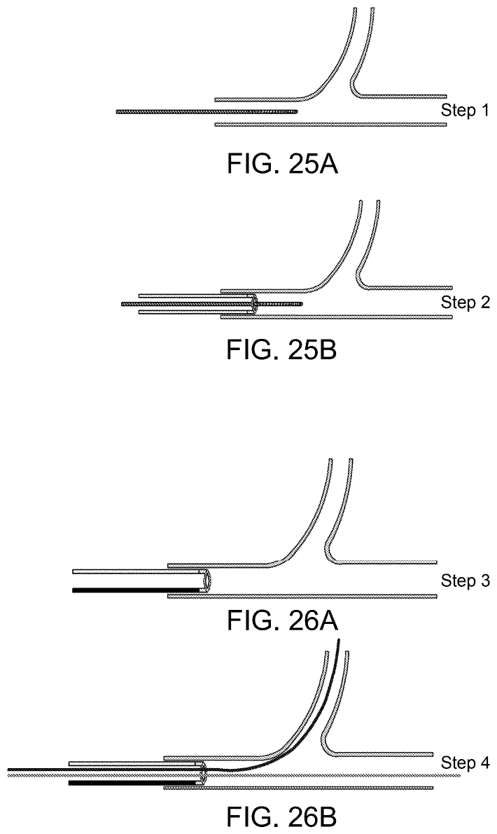

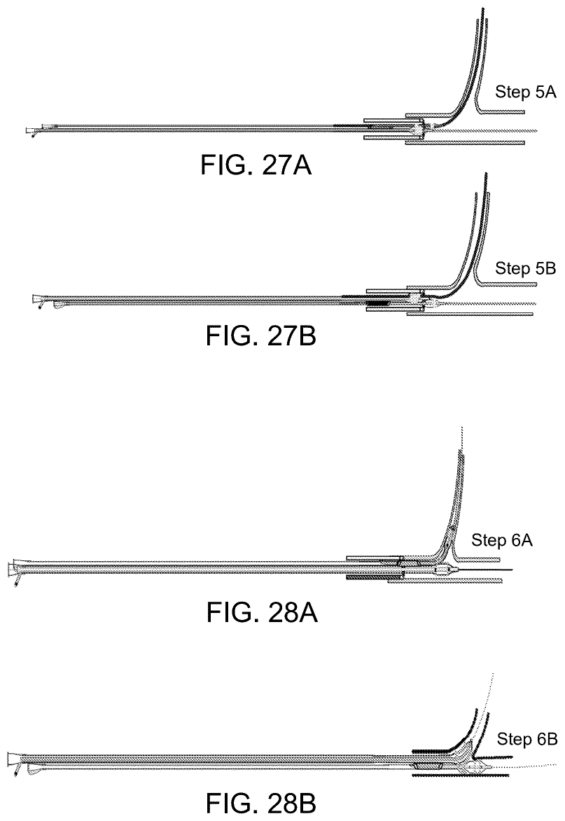

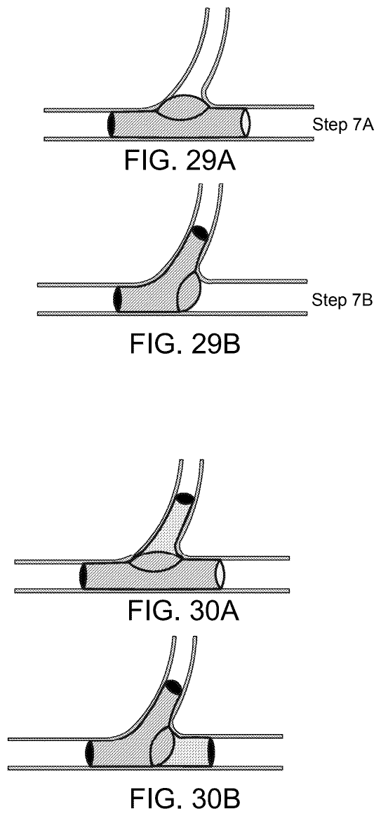

FIGS. 25A-30B illustrate an example of a delivery sequence in eight steps. Step 1 illustrates the introduction of a 0.035 inch guidewire up to the bifurcation. Step 2 illustrates the tracking of a guide catheter over the guidewire. Step 3 illustrates the removal of the guidewire and placement position of the guide catheter. Step 4 illustrates the tracking and placement of a rapid-exchange compatible wire in the daughter vessel and an over-the-wire compatible wire in the mother vessel. Steps 5A & 5B illustrate tracking of the catheter system distally over both the guidewires. Step 6A illustrates the inflation of the daughter balloon and placement of the daughter stent and partial deployment of the mother stent. Step 6B illustrates the inflation of the mother balloon to place the distal portion of the mother stent in the mother vessel. Step 7A illustrates the mother stent in the main branch with side hole facing the daughter vessel. Step 7B illustrates the bifurcated stent partially in the daughter vessel and daughter ostium completely opened and continuing on to the mother vessel.

In an alternative example, the delivery catheter mother balloons have tapered ends to accommodate balloons and stents with non-uniform profiles. For example, the proximal end of the daughter vessel stent may be designed to have a larger circumference than the distal end to compensate for the natural bifurcation anatomy. The daughter vessel balloon would likewise have a taper to properly expand the stent and ensure complete apposition. Additionally, it is possible to design the mother stent to expand differentially along its profile to compensate for a larger arterial diameter at the carina or ostium. In other words, the proximal and distal ends of the mother vessel balloon and mother vessel stent would be smaller in circumference while the center portion of the mother vessel stent would have a larger circumference. In an alternative example, the mother vessel balloon has tapered ends to accommodate the distal balloon catheter portion and guidewire lumen. Further, the mother vessel balloon may be designed for differential expansion to accommodate natural vessel anatomy.

In an example, the distal (daughter) balloon catheter portion is crimped with a half stent on a rapid-exchange catheter. The daughter vessel stent is about 4-20 millimeters long and the daughter vessel balloon is approximately twice as long in length. The mother vessel stent is about 10-30 millimeters long, and is differentially crimped to allow independent operation of the daughter balloon catheter portion. The distal portion of the mother vessel stent is crimped tightly enough to keep the entire stent from unintentionally dislodging during the procedure. The proximal portion of the mother vessel stent is crimped just tightly enough to reduce the crossing profile and to allow the daughter balloon catheter portion to be moved distal or proximal relative to the mother balloon catheter portion. The proximal (mother) balloon catheter portion is an over-the-wire type design with the mother vessel balloon optionally about 3 centimeters proximal to the daughter vessel balloon. In an alternative example, a stent is designed to allow differential expansion of the middle portion of the stent relative to the proximal and distal ends. In particular, the design facilitates the placement of the stent across a bifurcation lesion in the mother vessel because it has a larger circumference in the middle portion relative to the ends than a stent with a constant profile. Further, the profile can be adjusted so that the largest circumference can be placed proximal or distal to the midpoint of the stent. In the particular example, the largest circumference is distal to the midpoint of the stent, but could be easily reversed for variable patient anatomy. Partial crimping has the following features that make it possible to maintain sufficient stent retention during delivery and placement and still allows the secondary system adjustability and deliverability.

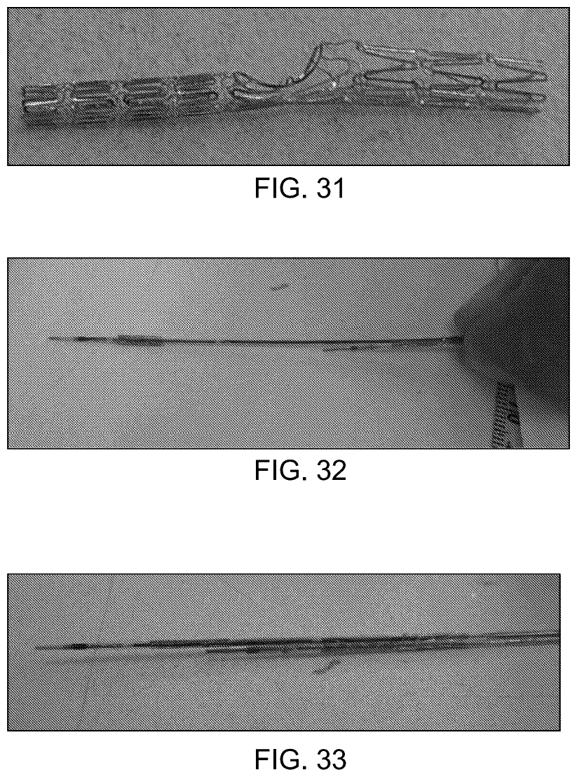

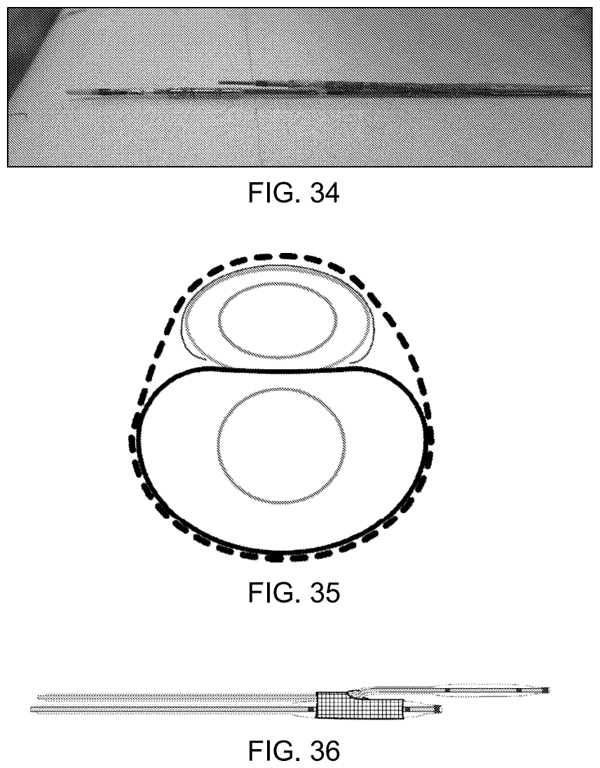

FIG. 31 shows a partially crimped bifurcation stent prior to placement on any balloon catheter. FIGS. 32-34 illustrate an example of the present inventive subject matter in three steps. First, the bifurcation stent is partially crimped over approximately one-third of its distal portion onto the mother catheter balloon, and the daughter catheter is loaded through the mother catheter and mother stent where the daughter stent can be crimped separately. Second, the daughter stent is crimped and pulled back proximally to align the daughter stent proximal end near the mother stent distal end. Third and final, the proximal portion of the mother stent can be crimped to reduce the outer diameter yet still allow independent movement of the two catheters relative to each other.

FIG. 35 illustrates a cross-section of a mother and daughter balloon catheter system without a daughter stent. The daughter catheter is on top of the mother catheter. The mother stent is differentially crimped around the mother catheter balloon and daughter catheter because the daughter catheter profile is smaller than the mother catheter. The differential crimping is non-uniform and can create various cross-sectional shapes to accommodate different catheter designs, balloon designs, and stent designs. For example, pear-shaped or a figure-eight are possible configurations. The current example is designed to reduce the profile as much as possible. In one example of a method of manufacturing, a protective sheet is placed between the two catheters. The protective sheet only needs to cover the portions that will come in contact during the crimping process; then the protective sheet can be removed.

FIG. 36 illustrates a side view of the mother stent mounted on the mother catheter balloon and the daughter catheter mounted on the mother catheter through the mother stent. The distal portion of the mother stent will be crimped under standard conditions to hold the stent firmly to the mother balloon and mother catheter. The proximal portion of the mother stent is partially crimped to reduce the profile but still allows the daughter catheter freedom to move proximal or distal relative to the mother catheter. This example illustrates that the stent is differentially crimped in both the circumferential and longitudinal directions. The amount of crimping will be determined by the stent design and size, catheter dimensions, and balloon dimensions; thus the crimping is differential along the longitudinal axis.

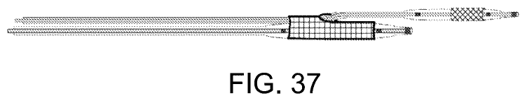

FIG. 37 illustrates a side view of the mother stent mounted on the mother catheter balloon and the daughter catheter mounted on the mother catheter through the mother stent. The daughter catheter also includes a stent that can be crimped under standard conditions. The distal portion of the mother stent will be crimped under standard conditions to hold the stent firmly to the mother balloon and mother catheter. In one experiment, this arrangement was tested to determine the strength of the distal crimping of the mother stent by pulling the daughter catheter and stent proximally; the results were that the daughter catheter successfully passed through the crimped mother stent and still retained the daughter stent as well. Additional features may be utilized during the crimping process such as adding a slight positive internal pressure to the balloon so that the final balloon surface pillows about 0.002 inch beyond the outer diameter of the stent. This process can yield a design that protects the stent from engaging with the vessel, thus reducing friction and improving stent retention at the same time.

Further, this process improves safety and reduces trauma to the vessel. While the above example discloses a bifurcation stent that is crimped at or about its distal half, this is not a limitation. The stent could be differentially crimped along its axis depending upon stent design, such as, for example, if a hole in the side of a stent was not centered along the axis. It may be desirable to have the distal crimped portion of the bifurcation stent extend just distal of the hole that the daughter catheter passes through. Alternatively, the distal crimped portion could extend partially or entirely over the hole that the daughter catheter passes through.

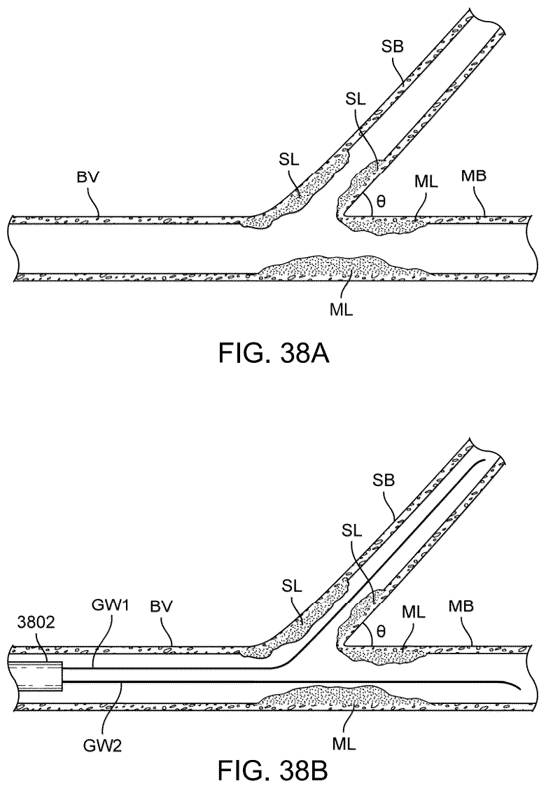

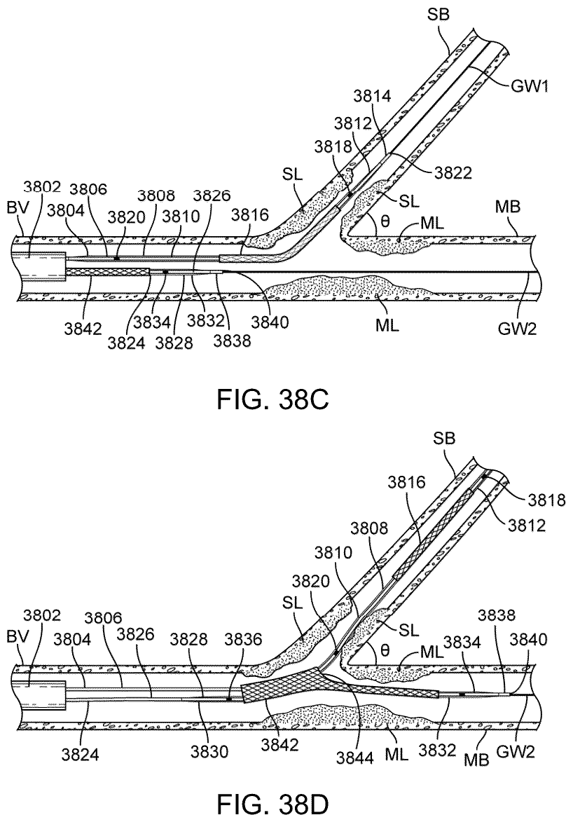

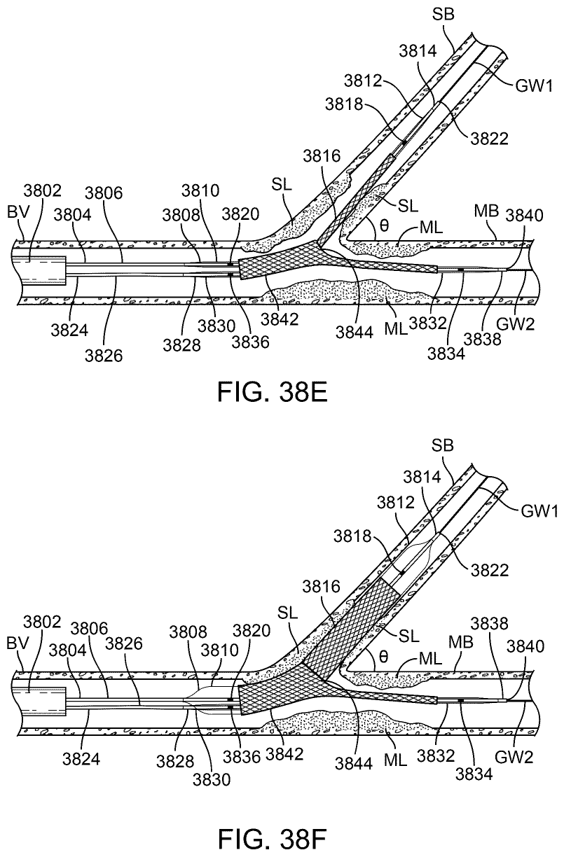

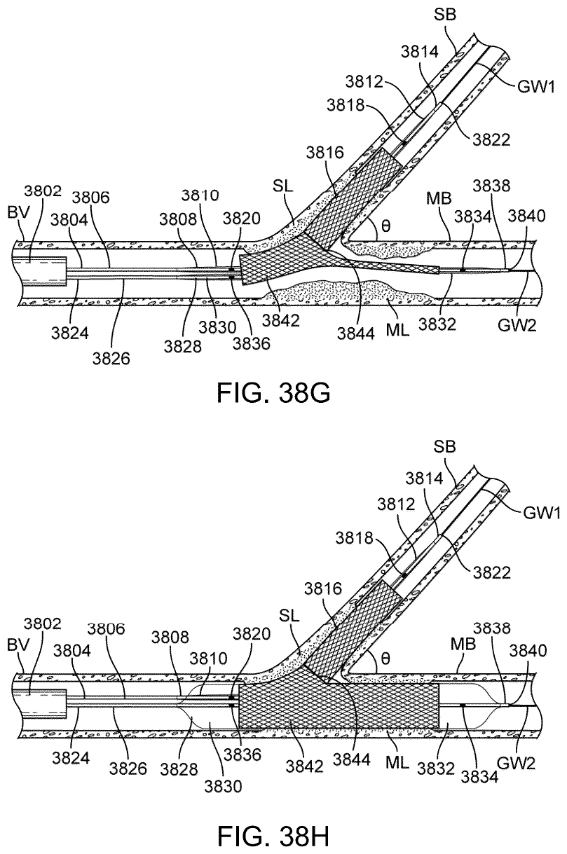

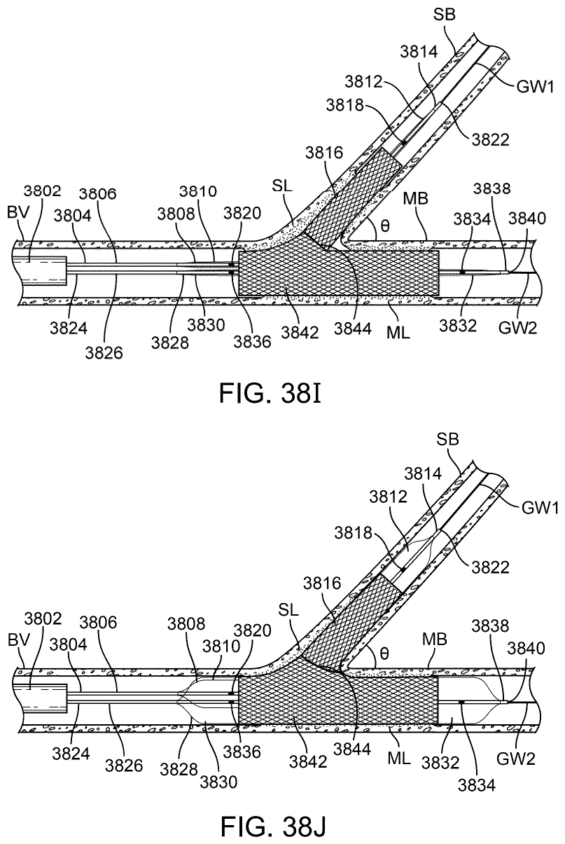

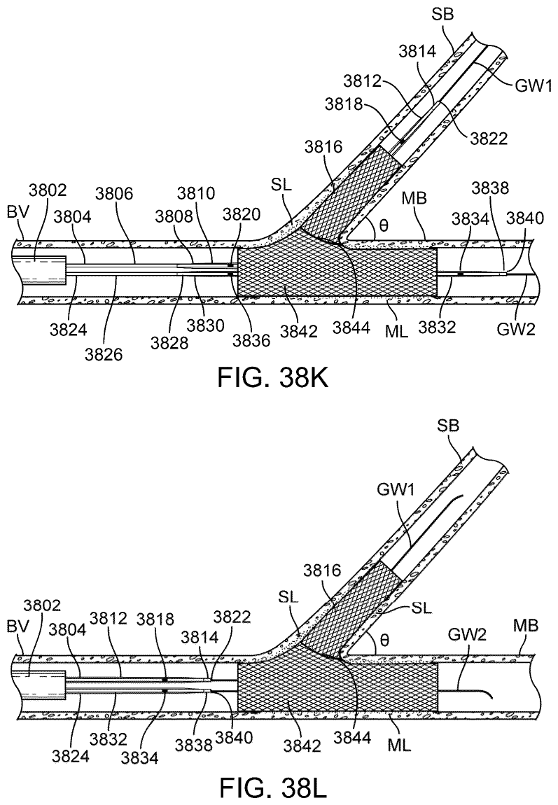

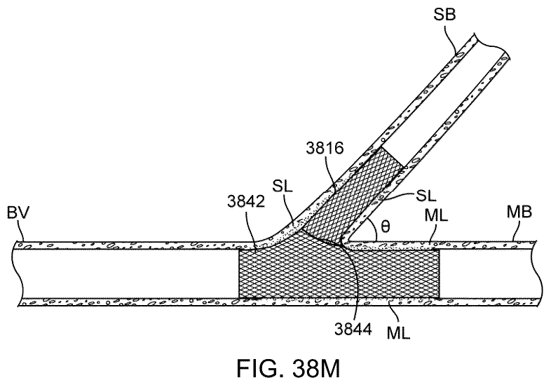

FIGS. 38A-38M more clearly illustrate an example of a method of treating a bifurcated vessel such as a bifurcated coronary artery. In FIG. 38A the bifurcated vessel BV includes a side branch vessel SB and a main branch vessel MB. The main branch has a main branch lesion ML, and the side branch has a side branch lesion SL. The angle between the side branch and the main branch is referred to as the bifurcation angle, and is indicated by 0. When the bifurcation angle θ is less than about 60 to 70 degrees, the distal-most stent of the system can be effectively positioned in the side branch. However, when the bifurcation angle is greater than or equal to about 60 to 70 degrees, it becomes more challenging to position the distal-most stent in the side branch. Moreover, when the distal stent is retracted proximally toward the stent having the side hole (discussed below), the catheter shaft may bind against the side hole, resulting in damage to the catheter shaft and/or stent. Therefore, in some examples, when the bifurcation angle is less than about 60 to 70 degrees, the distal-most stent may be positioned in the side branch and the proximal-most stent is advanced into the main branch. When the bifurcation angle is greater than or equal to about 60 to 70 degrees, the distal-most stent may be positioned in the main branch and the other stent is positioned partially in the main branch and partially in the side branch. This is not intended to limit the use of the catheter system, and either stent may be placed in either side branch or main branch depending on operator

In FIG. 38B, a guide catheter 3802 is advanced distally until its distal end is adjacent the bifurcation. A pair of guidewires GW1, GW2 are then advanced from the guide catheter 3802 distally toward the bifurcation such that the first guidewire GW1 is advanced into the side branch SB and so that the distal tip of the first guidewire GW1 is distal of the side branch lesion SL. Similarly, the second guidewire GW2 is also advanced distally in the main branch MB until the distal tip of the second guidewire GW2 is distal of the main branch lesion ML.

In FIG. 38C, a stent delivery system having a first catheter 3804 and a second catheter 3824 are advanced distally from the guide catheter 3802 toward the bifurcation. The first catheter 3804 includes an elongate catheter shaft 3806 and a radially expandable balloon 3808 disposed over a distal portion of elongate shaft 3806. A balloon-expandable stent 3816 is disposed over the balloon 3808. In this example, the stent 3816 is shorter than the working length of the balloon 3808; therefore, a proximal portion 3810 of the balloon 3808 and a distal portion 3812 are unconstrained by the stent 3816. The proximal portion 3810 may be retracted under a portion of the second stent 3842 and thus when balloon 3808 is inflated, it will radially expand stent 3816 and a portion of stent 3842. However, this is not intended to be limiting, and the stent length may be substantially equal to the working length of the balloon 3808, or it may have shorter length as previously discussed. Proximal radiopaque marker 3820 and distal radiopaque marker 3818 help define proximal and distal ends of the stent 3816 as well as proximal and distal ends of the balloon 3808. The radiopaque markers 3820, 3818 will also be used to help align the two catheters during treatment of the bifurcation, as will be discussed below. The distal tip 3814 may be a soft durometer polymer thereby minimizing trauma to the vessel during delivery. A distal guidewire port 3822 extends from the distal tip 3814 and allows guidewire GW1 to exit or enter a guidewire lumen (not shown) in the elongate shaft 3806. The first catheter 3804 may be a rapid exchange catheter or an over-the-wire catheter, examples of which have been disclosed above. The second catheter 3824 (best seen in FIG. 38D) includes an elongate catheter shaft 3826 with a radially expandable balloon 3828 disposed over a distal region of the elongate shaft 3826. A stent 3842 having a side hole 3844 is disposed over the balloon 3828. The length of the stent 3842 may be substantially the same as the working length of the balloon 3828 or it may be less than the working length. In this example, the stent 3842 has a length shorter than the working length of the balloon 3828, thus a proximal portion 3830 and a distal portion 3832 remain unconstrained by the stent 3842. Proximal radiopaque marker 3836 and distal radiopaque marker 3834 help define the proximal and distal ends of the stent 3842 as well as the proximal and distal ends of the balloon 3828. The radiopaque markers 3836, 3834 will also be used to help align the two catheters during treatment of the bifurcation, as will be discussed below. The distal tip 3838 may be a soft durometer polymer thereby minimizing trauma to the vessel during delivery. A distal guidewire port 3840 extends from the distal tip 3838 and allows guidewire GW2 to exit or enter a guidewire lumen (not shown) in the elongate shaft 3826. The second catheter 3824 may be a rapid exchange catheter or an over-the-wire catheter, examples of which have previously been disclosed above.

Referring back to FIG. 38C, the bifurcation angle is less than about 60 to 70 degrees, and the first catheter 3804 and the second catheter 3824 are further advanced distally so that the first catheter 3804 tracks over the first guidewire GW1 into the side branch SB while the second catheter 3824 tracks over the second guidewire GW2 in the main branch MB toward the main branch lesion ML. Because the first catheter 3804 is coupled with the second catheter 3824 via stent 3842, both catheters are advanced distally simultaneously thereby reducing procedure time, although this is not meant to be limiting, as each catheter may be advanced independently of the other. In this example, the first balloon 3808 and first stent 3816 are distal to the second balloon 3828 and second stent 3842. This axial offset minimizes the system profile.

In FIG. 38D, both catheters 3804, 3824 are advanced further distally toward the bifurcation until the first stent 3816 is distal to the side branch lesion SL and the second stent 3842 traverses the main branch lesion ML and the side hole 3844 is adjacent the ostium of the side branch SB. Advancement of both catheters 3804, 3824 is again performed simultaneously, although they could also be advanced independently of one another. The operator will feel resistance against further advancement of the catheters 3804, 3824 because as the catheters are advanced further distally, the two catheter shafts 3806, 3826 will spread apart relative to one another as they are forced against the carina of the bifurcation. However, a portion of the first elongate shaft 3806 is disposed under a portion of the second stent 3842, therefore the two shafts 3806, 3826 can only spread apart so far. Thus, when an operator feels resistance against further advancement of the catheter shafts, the operator knows that both catheters 3804, 3824 and their associated stents and balloons are properly positioned relative to the bifurcation.

In FIG. 38E, the first catheter 3804 is retracted proximally relative to the second catheter 3824. Because a portion of the first catheter shaft 3806 is disposed under a portion of the second stent 3842, the first shaft 3806 is slidably retracted into side hole 3844 and the first shaft 3806 and proximal portion 3810 of balloon 3808 are slidably retracted under a portion of second stent 3842. The first shaft 3806 is proximally retracted until proximal radiopaque marker 3820 lines up with proximal radiopaque marker 3836 so that a proximal end of the first stent 3816 will be aligned with the side hole 3844 in the second stent 3842. An operator may feel resistance during retraction of the first elongate shaft 3806 relative to the second elongate shaft 3826 when the ends of the stents 3816, 3842 engage one another. Stent 3842 has a distal portion crimped to balloon 3828 to prevent ejection during delivery, and a proximal portion is partially crimped thereto or uncrimped to allow catheter 3804 to slide thereunder. Crimping of the stent is disclosed in greater detail in U.S. patent applications previously incorporated by reference above. The ends of the stents may butt up against one another, overlap with one another, interleave with one another, or combinations thereof. Additional details related to the engagement of the stents is disclosed in U.S. patent applications previously incorporated by reference above. Both stents 3816, 3842 are disposed adjacent their respective lesions SL, ML, and the side hole 3844 is in rough alignment with the ostium to the side branch SB and the side branch stent 3816.

In FIG. 38F, the balloon 3808 is radially expanded, often with contrast medium, saline, or a combination thereof, thereby radially expanding the first stent 3816 into engagement with the side branch lesion SL and the walls of the side branch. A proximal portion 3810 and a distal portion 3812 of the balloon 3808 will also expand; thus a proximal portion of the second stent 3842 will also be radially expanded. Expansion of the stents occurs simultaneously. Since a portion of balloon 3808 also passes through side hole 3844, expansion of balloon 3808 also partially expands the side hole 3844 and also aligns the side hole 3844 with the ostium of the side branch.

In FIG. 38G the balloon 3808 is contracted, and then in FIG. 38H the other balloon 3828 is radially expanded, with contrast medium, saline, or a combination thereof, thereby further radially expanding the second stent 3842. Expansion of balloon 3828 expands the proximal portion of the stent 3842 into engagement with the main branch vessel wall and main branch lesion ML, and the distal portion of the stent 3842 is also radially expanded into the main branch vessel wall as well as the main branch lesion ML. The side hole 3844 is also further aligned with the ostium of the side branch SB.

Referring now to FIG. 38I, balloon 3828 is contracted and then both balloons are simultaneously inflated in a “kissing balloon” technique as seen in FIG. 38J. Both balloons 3808, 3828 are inflated with contrast medium, saline, or combinations thereof until they engage one another and are fully expanded in the main branch MB and side branch SB. The kissing balloon technique ensures that both stents 3816, 3842 are fully expanded and in full apposition with their respective vessel wall and lesion. Additionally, the kissing balloon technique lines up the proximal end of the first stent 3816 with the side hole 3844 in the second stent 3842, thereby ensuring that continuous and smooth scaffolding from the main branch MB into the side branch SB. Also, the kissing balloons technique ensures that the side hole 3844 does not block the ostium to the side branch thereby avoiding “stent jailing,” or disrupting blood flow into the side branch.

In FIG. 38K, both balloons 3808, 3828 are contracted, and in FIG. 38L both catheters 3804, 3824 are retracted proximally. The catheters 3804, 3824 may be retracted simultaneously or independently of one another. The first catheter 3804 is retracted through both stents 3816, 3842 and also passes through the side hole 3844. The second catheter 3824 is retracted through the second stent 3842. In FIG. 38M, both catheters 3804, 3824 have been removed, as well as the guide catheter 3802 and both guidewires GW1, GW2. Stents 3816, 3842 remain implanted at the bifurcation. Optionally, the stents or balloons may contain therapeutic agents such as those previously discussed, and these may elute out into the lesion at a controlled rate in order to help prevent restenosis.

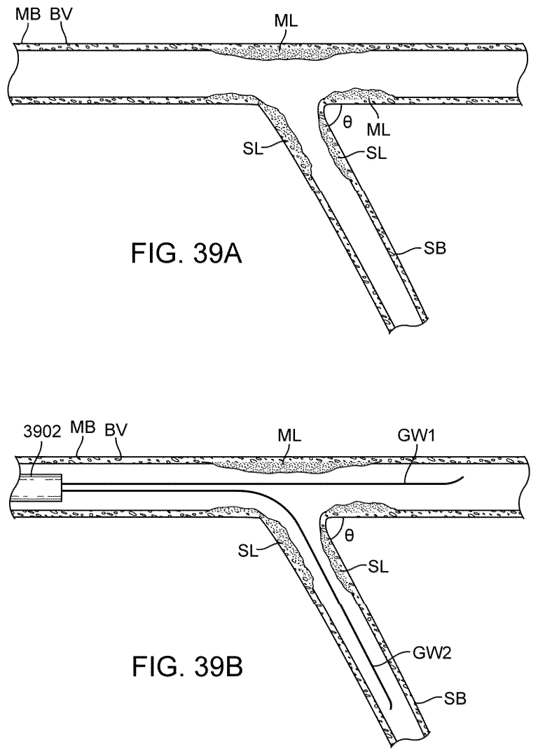

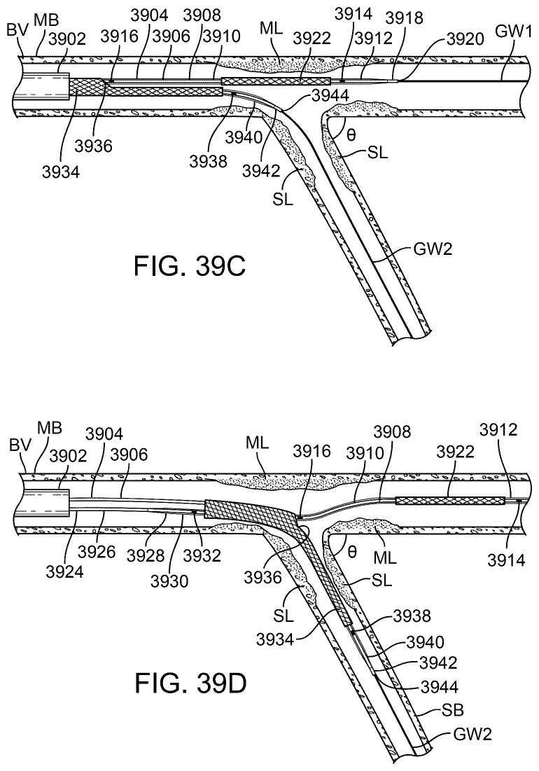

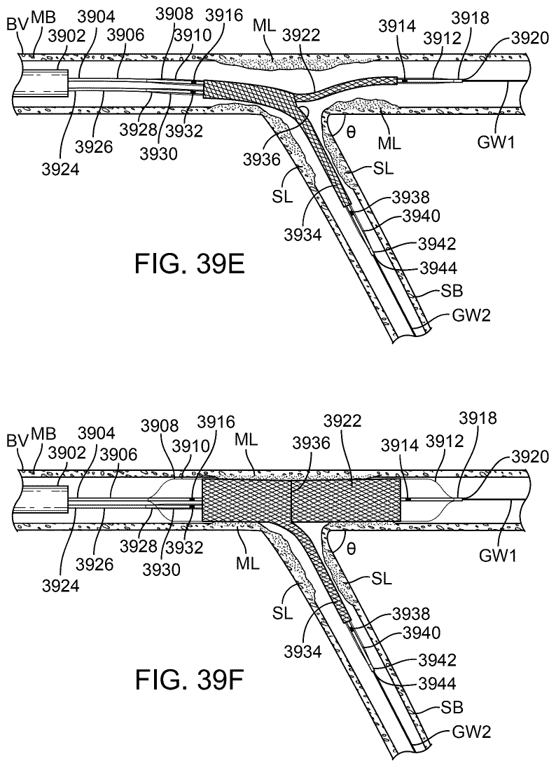

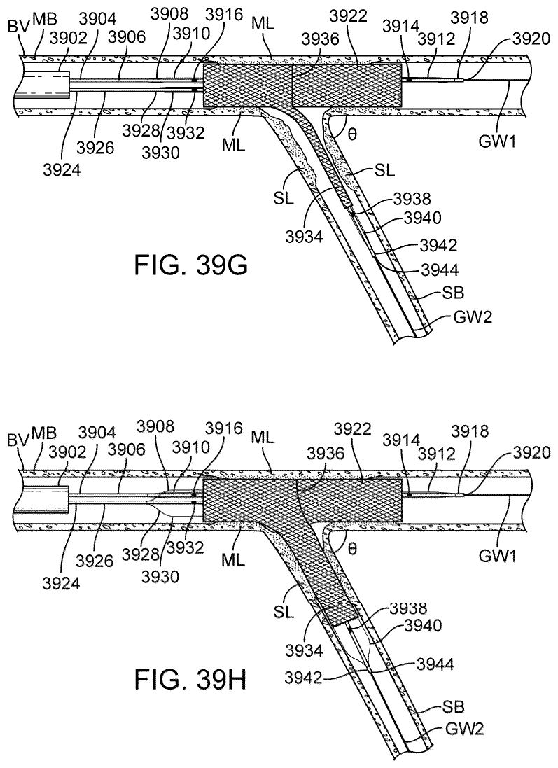

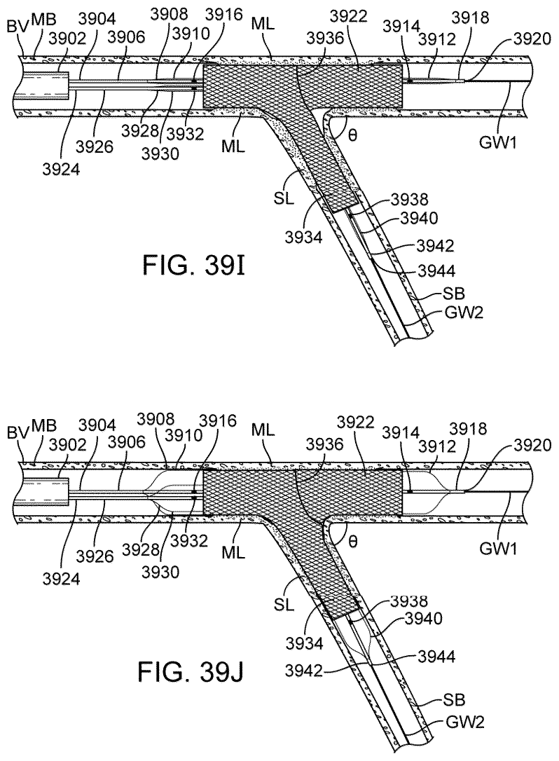

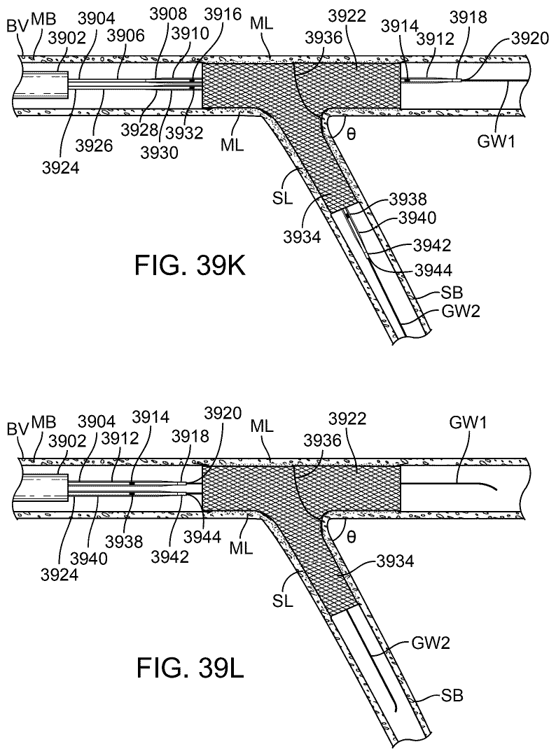

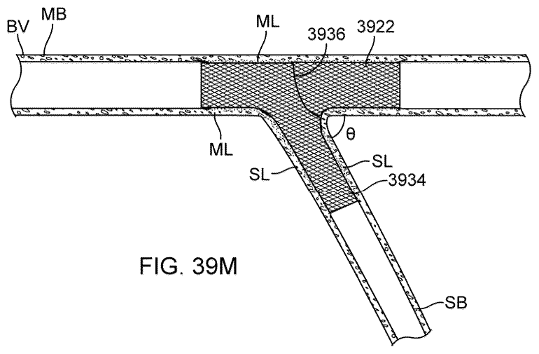

FIGS. 39A-39M more clearly illustrate another example of a method for treating a bifurcated vessel. This method is similar to that previously disclosed, with the major difference being that the distal-most catheter is used to treat the main branch vessel, and the proximal-most catheter is used to treat the side branch vessel. In the previous example, the distal-most catheter is used to treat the side branch vessel and the proximal-most catheter is used to treat the main branch.

In FIG. 39A, the bifurcated vessel BV includes a side branch vessel SB and a main branch vessel MB. The main branch has a main branch lesion ML, and the side branch has a side branch lesion SL. The angle between the side branch and the main branch is referred to as the bifurcation angle, and is indicated by 0. When the bifurcation angle θ is less than about 60 to 70 degrees, the distal-most stent of the system can be effectively positioned in the side branch. However, when the bifurcation angle is greater than or equal to about 60 to 70 degrees, it becomes more challenging to position the distal-most stent in the side branch. Moreover, when the distal stent is retracted proximally toward the stent having the side hole (discussed below), the catheter shaft may bind against the side hole resulting in damage to the catheter shaft and/or stent. Therefore, in some examples, when the bifurcation angle is less than about 60 to 70 degrees, the distal-most stent may be positioned in the side branch and the proximal-most stent is advanced into the main branch. When the bifurcation angle is greater than or equal to about 60 to 70 degrees, the distal-most stent is positioned in the main branch and the other stent is positioned partially in the main branch and partially in the side branch. This is not intended to limit the use of the catheter system, and either stent may be placed in either side branch or main branch depending on operator preference. In FIG. 39B, a guide catheter 3902 is advanced distally into the vessel until it is adjacent the bifurcation and the lesions ML, SL. A first guidewire GW1 is advanced distally in the main branch MB until it is distal of the main branch lesion ML. A second guidewire GW2 is also advanced distally until it enters the side branch SB and it is distal of the side branch lesion SL.

In FIG. 39C, a treatment system having a first catheter 3904 and a second catheter 3924 (best seen in FIG. 39D) are advanced distally through the guide catheter 3902 toward the bifurcation. The two catheters 3904, 3924 may be advanced independently of one another, or the two catheters 3904, 3924 may be advanced simultaneously. The first catheter 3904 includes an elongate shaft 3906 with a radially expandable balloon 3908 on a distal portion of the elongate shaft 3906. A stent 3922 is disposed over the balloon 3908. The length of the stent 3922 may substantially match the working length of the balloon 3908, or the length of the stent 3922 may be less than the working length of the balloon 3908 such that a proximal portion 3910 and a distal portion 3912 of the balloon 3908 remains unconstrained by the stent 3922. A proximal radiopaque marker 3916 and a distal radiopaque marker 3914 may be used to help determine the proximal and distal ends of the balloon 3908 as well as the proximal and distal ends of the stent 3922. A soft durometer polymer tip may be used on the distal portion of the catheter shaft 3906 so as to prevent trauma to the vessel during delivery, and the catheter shaft 3906 has a distal guidewire port 3920 to allow a guidewire GW1 to enter or exit a guidewire lumen (not shown) in the catheter shaft 3906. The first catheter 3904 may be a rapid-exchange catheter or it may be an over-the-wire catheter. The second catheter 3924 (best seen in FIG. 39D) includes an elongate shaft 3926 having a radially expandable balloon 3928 on a distal portion thereof. A second stent 3934 is disposed over the second balloon 3928. The stent length may substantially match the working length of the balloon 3928, or it may be less. In this example, the length of stent 3934 is less than the working length of balloon 3928; thus a proximal portion 3930 and a distal portion 3940 of the balloon 3938 remain unconstrained by the stent 3934. A portion of the first elongate shaft 3906 is disposed under a proximal portion of the second stent 3934, and the stent 3934 also has a side hole 3936 so that the first elongate shaft 3906 may exit therefrom. The first elongate shaft 3906 may slide under the stent 3934 relative to the second elongate shaft 3926; thus a proximal portion 3910 of balloon 3908 is also disposed under stent 3934. When balloon 3908 is expanded, a proximal portion of stent 3934 will also be expanded. The second elongate shaft 3926 also includes a proximal radiopaque marker 3932 and a distal radiopaque marker 3938 that help identify the proximal and distal ends of the balloon 3928 and the proximal and distal ends of the stent 3934. The second catheter 3924 also has a soft durometer polymer tip 3942 that helps minimize trauma to the vessel during delivery, and a distal guidewire port 3944 allows a guidewire to be inserted or to exit from a guidewire lumen (not shown) in the elongate shaft 3926. The second catheter 3924 may be an over-the-wire catheter or it may be rapid exchange. The first stent 3922 and balloon 3908 are distal to the second stent 3939 and second balloon 3928.

In FIG. 39D, the bifurcation angle θ is greater than about 60 to 70 degrees. Both catheters 3904, 3924 are further advanced distally toward the bifurcation until the first stent 3922 is distal to the main branch lesion ML, and the second stent 3934 is partially disposed in the side branch SB adjacent the side branch lesion SL, and the stent 3934 is also disposed in the main branch MB adjacent the main branch lesion ML. The side hole 3936 also faces generally in the direction of the main branch vessel MB. Advancement of both catheters 3904, 3924 may be performed simultaneously, although they could also be advanced independently of one another. The operator will feel resistance against further advancement of the catheters 3904, 3924 because as the catheters are advanced further distally, the two catheter shafts 3906, 3926 will spread apart relative to one another as they are forced against the carina of the bifurcation. However, a portion of the first elongate shaft 3906 is disposed under a portion of the second stent 3934, therefore the two shafts 3906, 3926 can only spread apart so far. Thus, when an operator feels resistance against further advancement of the catheter shafts, the operator knows that both catheters 3904, 3924 and their associated stents and balloons are properly positioned relative to the bifurcation.

In FIG. 39E the first catheter 3904 is retracted proximally relative to the second catheter 3924 so a proximal portion 3910 of balloon 3908 is disposed under stent 3934. Stent 3934 has a distal portion crimped to balloon 3928 so that it will not be ejected during delivery, and a proximal portion is partially crimped or uncrimped over balloon 3928 to allow shaft 3906 to slidably pass thereunder. Stent crimping is described in greater detail in U.S. patent applications previously incorporated by reference above. Because a portion of the first catheter shaft 3906 is disposed under a portion of the second stent 3934, the first shaft 3906 is slidably retracted into side hole 3936 and the first shaft 3906 is also slidably retracted under a portion of second stent 3934. The first shaft 3906 is proximally retracted until proximal radiopaque marker 3916 lines up with proximal radiopaque marker 3932 so that a proximal end of the first stent 3922 will be aligned with the side hole 3936 in the second stent 3934. An operator may feel resistance during retraction of the first elongate shaft 3906 relative to the second elongate shaft 3926 when the ends of the stents 3922, 3934 engage one another. The ends of the stents 3922, 3934 may butt up against one another, overlap with one another, interleave with one another, or combinations thereof. Additional details related to the engagement of the stents are disclosed in U.S. patent applications previously incorporated by reference above. Both stents 3922, 3934 are disposed adjacent their respective lesions SL, ML, and the side hole 3936 is in rough alignment with the main branch vessel MB.

In FIG. 39F, the balloon 3908 is radially expanded, often with contrast medium, saline, or a combination thereof thereby radially expanding the first stent 3922 into engagement with the main branch lesion ML and the walls of the main branch. A proximal portion of the second stent 3934 is also expanded into engagement with the main branch lesion ML and the walls of the main branch, while a distal portion of the second stent 3934 remains unexpanded in the side branch SB. The first stent 3922 and the proximal portion of the second stent 3934 are radially expanded simultaneously. The inner surfaces of both stents form a smooth lumen for blood flow through the main branch. Since a portion of balloon 3908 also passes through side hole 3936, expansion of balloon 3908 also partially expands the side hole 3936 and also aligns the side hole 3936 with the main branch lumen.

In FIG. 39G the balloon 3908 is contracted, and then in FIG. 39H the other balloon 3928 is radially expanded, with contrast medium, saline, or a combination thereof, thereby further radially expanding the second stent 3934. Expansion of balloon 3928 expands a distal portion of stent 3934 into engagement with the side branch vessel wall and side branch lesion SL. The proximal portion of stent 3934 and side hole 3936 may also be further expanded and aligned with the first stent 3922. The side hole 3936 is also further aligned with the lumen of the main branch.

Referring now to FIG. 39I, balloon 3928 is contracted and then both balloons are simultaneously inflated in a “kissing balloon” technique as seen in FIG. 39J. Both balloons 3908, 3928 are inflated with contrast medium, saline, or combinations thereof until they engage one another and are fully expanded in the main branch MB and side branch SB. The kissing balloon technique ensures that both stents 3922, 3934 are fully expanded and in full apposition with their respective vessel wall and lesion. Additionally, the kissing balloon technique lines up the proximal end of the first stent 3922 with the side hole 3936 in the second stent 3934, thereby ensuring that continuous and smooth scaffolding from the main branch MB into the side branch SB. Alignment of the two stents is disclosed in greater detail in U.S. patent applications previously incorporated by reference above. Also, the kissing balloons technique ensures that the side hole does not block the main branch or disrupting blood flow across the bifurcation.

In FIG. 39K, both balloons 3908, 3928 are contracted, and in FIG. 39L both catheters 3904, 3924 are retracted proximally. The catheters may be retracted simultaneously or independently of one another. The first catheter 3904 is retracted through both stents 3922, 3934 and also passes through the side hole 3936. The second catheter 3924 is retracted through the second stent 3934. In FIG. 39M, both catheters 3904, 3924 have been removed, as well as the guide catheter 3802 and both guidewires GW1, GW2. Stents 3922, 3934 remain implanted at the bifurcation. Optionally, the stents or balloons may contain therapeutic agents such as those previously discussed, and these may elute out into the lesion at a controlled rate in order to help prevent restenosis.

Any of the methods described above may use any of the stents disclosed herein in any of the system configurations described. Additionally, any of the features previously described above may also be used. Therefore, one of skill in the art will appreciate that any number of combinations may be made. For example, catheter systems may have any combination of rapid exchange or over-the-wire configurations, with any of the stents disclosed herein, with or without a therapeutic agent on a stent or a balloon, and with or without any of the hollow exchange port, capture tube, removable capture tube, or snap fittings described above.

### Stents:

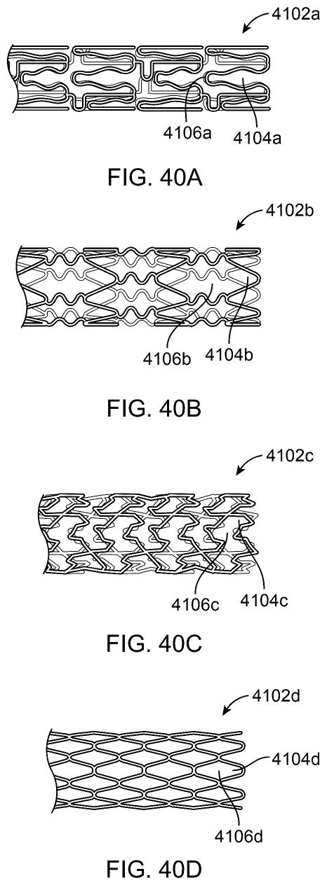

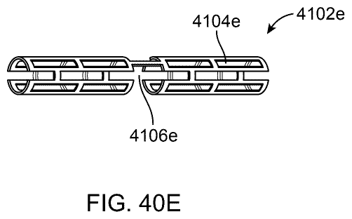

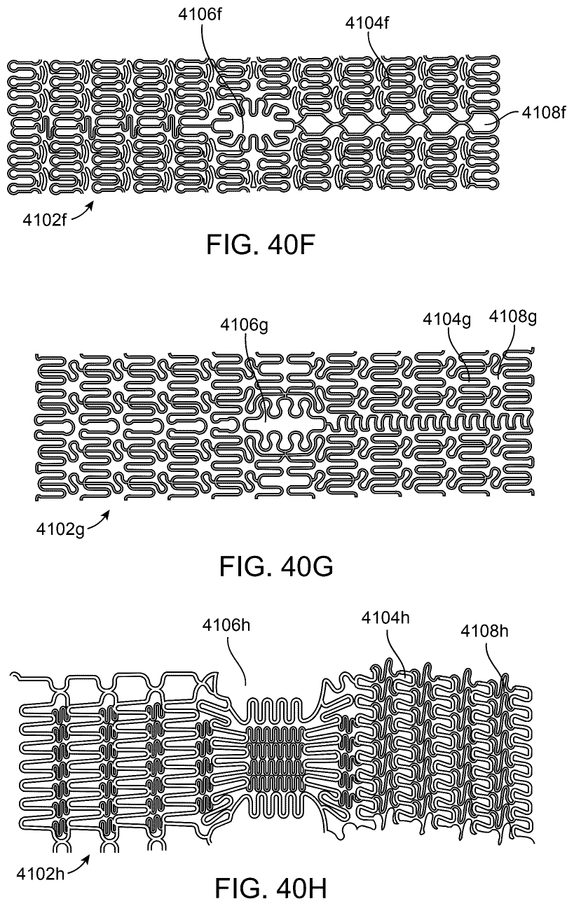

The catheter systems and methods described above may use a commercially available stent for either the proximal or distal stent in the system. When a commercially available stent is used for the distal stent, it need only be crimped to the distal balloon catheter. When the commercially available stent is used for the proximal stent it may be partially crimped to the proximal balloon such that a portion of a second catheter shaft is slidably disposed under the stent and a portion of the second catheter shaft slidably passes through a side hole in the stent. The stent is crimped to the proximal balloon so that it is not displaced from the balloon during delivery, and also so the second catheter shaft can slide thereunder. FIGS. 40A-40E illustrate several examples of commercially available stents that may be used in catheter system configurations and methods described above, either as is, or with slight modification. For example, FIG. 40A illustrates the Abbott Vascular Xience® drug eluting stent 4102a. A portion of a catheter shaft may be disposed under the stent 4102a through its central channel and the catheter may exit a side hole in the stent 4102a. A side hole may be the gap 4104a created between adjacent struts in a cell, or the gap 4106a between axially adjacent cells. FIG. 40B illustrates the Cordis Cypher® stent 4102b. Again a portion of a catheter shaft may be disposed under the stent 4102b through its central channel and the catheter may exit a side hole in the stent 4102b. A side hole may be the gap 4104b created between adjacent struts in a cell, or the gap 4106b between axially adjacent cells. FIG. 40C illustrates the Boston Scientific Taxus® Liberte® stent 4102c. A portion of a catheter shaft may be disposed under the stent 4102c through its central channel and the catheter may exit a side hole in the stent 4102c. A side hole may be the gap 4104c created between adjacent struts in a cell, or the gap 4106c between axially adjacent cells. FIG. 40D illustrates the Medtronic Endeavor® stent 4102d. A portion of a catheter shaft may be disposed under the stent 4102d through its central channel and the catheter may exit a side hole in the stent 4102d. A side hole may be the gap 4104d created between adjacent struts in a cell, or the gap 4106d between axially adjacent cells. FIG. 40E illustrates a Palmaz-Schatz® stent 4102e. A portion of a catheter shaft may be disposed under the stent 4102e through its central channel and the catheter may exit a side hole in the stent 4102e. A side hole may be the gap 4104e created between adjacent struts in a cell, or the gap 4106e between axially adjacent segments.

Other stents have been designed with side holes that are specifically intended to treat bifurcations. These stents may also be used with the systems and method disclosed herein. For example, FIGS. 40E-40H illustrate several examples of stents from Boston Scientific and are disclosed in detail in U.S. Pat. No. 7,678,142. FIG. 40F shows a stent 4102f after it has been unrolled and flattened having a side hole 4106f. FIG. 40F illustrates a stent geometry (unrolled, plan view) where the struts create a side hole 4106f that allows access to a side branch, and that can accommodate a catheter shaft as described herein. The side hole 4106f may be formed by the spaces 4104f, 4108f between struts. FIG. 40G illustrates another stent geometry (unrolled, plan view) of stent 4102g having a side hole 4106g. Alternatively, the side hole 4106g may be formed by the spaces 4104g, 4108g between struts or axial connectors. FIG. 40H illustrates still another stent geometry (unrolled, plan view) of stent 4102h having a side hole 4106h. The side hole 4106h may also be formed by the space between struts 4104h or axial connectors 4108h. In any of these examples, a catheter shaft may be slidably disposed under a portion of the stent, and the catheter shaft may exit the side hole. Additionally, any of the stents or balloons disclosed herein may carry a therapeutic agent such as those described above for local drug delivery. Also, while the stents disclosed herein are often described as balloon expandable, one of skill in the art will appreciate that self-expanding and hybrid balloon expandable/self-expanding stents may also be used.

### Stent Alignment:

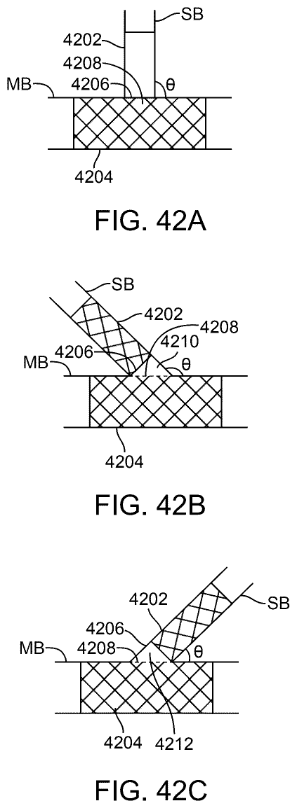

FIGS. 42A-42C illustrate various ways a side branch stent can line up with a main branch stent. In FIG. 42A, the side branch SB is substantially perpendicular to the main branch MB; therefore the bifurcation angle θ is about 90 degrees. In this situation, the proximal end 4206 of the side branch stent 4202 will be substantially flush with the side hole 4208 in the main branch stent 4204 (assuming proper deployment of both stents). This is desirable since there are no gaps and hence no unscaffolded regions between the two stents 4202, 4204. However, when the bifurcation angle θ increases (FIG. 42B) or decreases (FIG. 42C), a portion of the side branch will remain unstented. For example, in FIG. 42B the bifurcation angle increases, and because of the right cylindrical shape of the stent, in which the end is perpendicular to the sidewalls of the stent, a gap 4210 exits between the proximal end 4206 of the side branch stent 4202 and the side hole 4208 of the main branch stent 4204. Similarly, in FIG. 42C, when the bifurcation angle decreases, there is also a gap 4212 between the proximal end 4206 of stent 4202 and the side hole 4208 of stent 4204. FIG. 42C is typical of human anatomy, therefore the gap 4212 often is upstream of the bifurcation. Gaps are undesirable since they are unscaffolded and recoil, and restenosis may occur in this region. Additionally, in the case where a stent is used for drug elution, the gap region may not receive any of the drug.

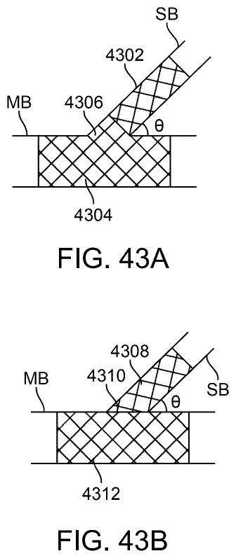

One possible solution for ensuring that the gap between a side branch stent and a main branch stent is eliminated or reduced is shown in FIG. 43A. The side branch stent 4302 is a right cylindrical stent. The main branch stent 4304 has a side hole 4306 with struts that expand outwardly into the gap region, thereby ensuring continuous scaffolding. An alternative solution in FIG. 43B is to fabricate the proximal end 4310 of the side branch stent 4308 with its proximal end non-perpendicular to the central axis of the stent so that the proximal end of the side branch stent lines up with the side hole in the main branch stent 4312. Even using the geometries illustrated in FIG. 43A-43B still requires careful alignment of the side branch stent with the main branch side hole. Therefore, it would be desirable to provide a stent geometry that facilitates alignment.

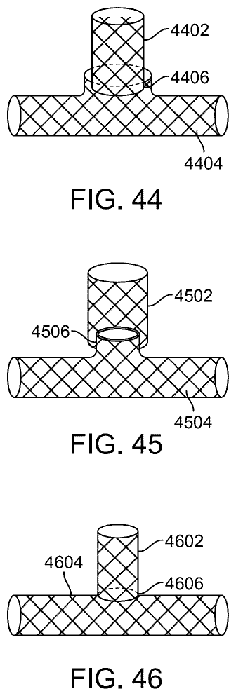

The ends of the side branch stent and the main branch stent may intersect in several different ways thereby providing continuous and uniform coverage of the bifurcation. For example, in FIG. 44, a portion 4406 of side branch stent 4402 may be disposed inside main branch stent 4404. FIG. 45 shows a portion 4506 of the main branch stent 4504 disposed inside the side branch stent 4502. Neither situation in FIG. 44 or 45 is ideal as overlapping of stents may result in metal rubbing on metal as well as possibly disrupting blood flow or causing stagnation points. A more desirable interface between stents is shown in FIG. 46 where the end of the side branch stent 4602 butts up against the side hole in main branch stent 4604. The interface region 4606 is desirable since it provides continuous scaffolding of the vessel without gaps between ends of the stents. However, depending on the stent geometry, gaps may still exist between stents. Therefore, in some examples, the ends of the stents will interleave or interdigitate with one another.

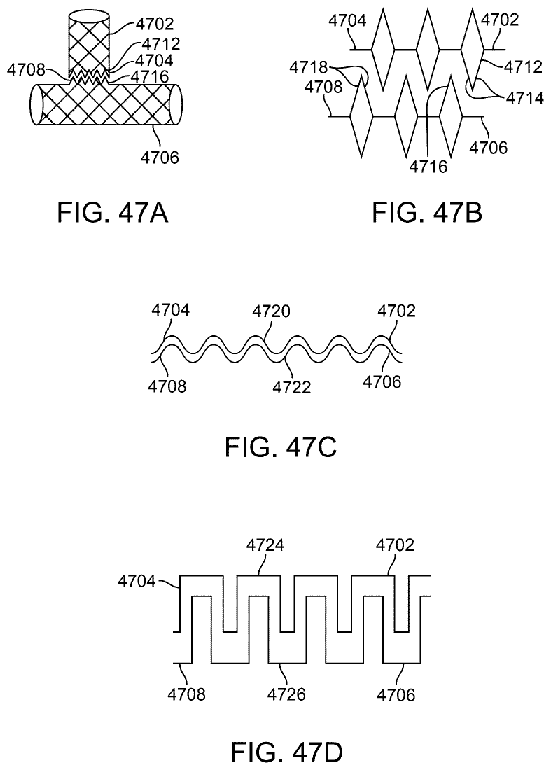

FIGS. 47A-47D illustrate several examples where the ends of the side branch stent and the side hole of the main branch stent interleave with one another or interdigitate. For example, in FIG. 47A, a proximal end 4704 of side branch stent 4702 has a series of axially extending elements or fingers 4712 which interdigitate or interleave with the laterally extending elements or fingers 4716 that extend laterally from the side hole 4708 of main branch stent 4706. FIG. 47B illustrates an example of interdigitating axial and lateral elements. A proximal end 4704 of side branch stent 4702 has a plurality of axially extending elements 4712. The axially extending elements 4712 are formed from a plurality of interconnected stent struts 4714, in this case forming a triangular shape. Similarly, the side hole 4708 of the main branch stent 4706 has a plurality of laterally extending elements 4716 that are formed from a plurality of interconnected stent struts 4718. In this case the laterally extending elements 4716 are formed into a triangular shape. Thus the apex of one triangular shaped element fits in between adjacent elements on the adjacent stent. Or alternatively, the peaks fit in the valleys, and the valleys receive the peaks.

FIG. 47C illustrates still another example of interleaving or interdigitating elements. The proximal end 4704 of the side branch stent 4702 includes a strut 4720 formed into a series of peaks and valleys. Similarly, the side hole 4708 of the main branch stent 4706 will also have a strut 4722 that has been formed into a series of peaks and valleys. Therefore, the peaks of the side branch stent 4702 will fit into the valleys of the adjacent main branch stent side hole, and similarly the valleys of the side branch stent 4702 receive the peaks of the side hole. FIG. 47D illustrates yet another example of interleaving or interdigitation of stent ends. The proximal end 4704 of side branch stent 4702 includes a strut 4724 formed into a series of rectangular peaks and valleys. The side hole 4708 of the main branch stent 4706 also has a strut 4726 formed into a series of rectangular peaks and valleys. The peaks and valleys interleave and interdigitate with one another.

### Balloon Configurations:

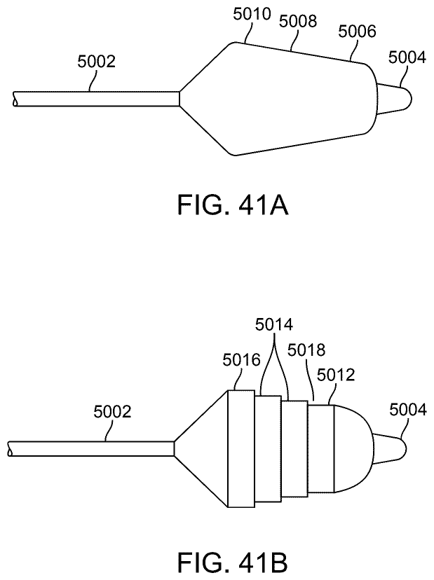

The balloons used to radially expand the stents described herein may be cylindrical balloons having a constant diameter along the working length, or diameter may vary. When stenting a tapered vessel, it may be beneficial to use a balloon which has a variable diameter balloon that more closely matches the vessel anatomy. For example, in FIG. 41A, a tapered balloon 5006 is attached to the distal portion of shaft 5002. A soft durometer tip 5004 prevents vessel trauma during delivery. The balloon 5006 is tapered such that a proximal portion 5010 of the balloon 5006 has a larger diameter than a distal portion. Any taper may be used. FIG. 41B illustrates another example of a balloon 5012 having a plurality of stepped regions 5014. The stepped regions 5014 may be incremented in any amount, and in some examples, a proximal portion 5016 of the balloon 5012 has a larger diameter than a distal portion 5018. Any of these examples, or combinations thereof, may be used in the systems and methods described herein to treat a bifurcation. Use of a tapered or stepped balloon allows a stent to be expanded to more closely match the vessel walls, where a proximal portion of the expanded stent has a larger diameter than a distal portion of the stent.

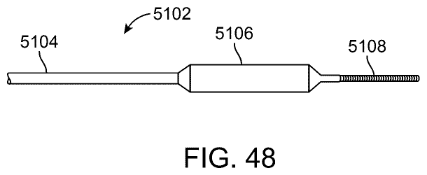

In addition to using catheters having rapid exchange or over-the-wire guidewire lumens, and tapered or stepped balloons, the balloon catheters may not always employ a guidewire lumen. Instead, a fixed wire may be attached to a distal end of the catheter. For example, FIG. 48 illustrates an example of a fixed wire catheter 5102 having a balloon 5106 attached to a distal portion of the shaft 5104. A section of guidewire 5108 is fixedly attached to the distal end of the catheter 5102 and this fixed wire helps the catheter 5102 track through the vessels. The fixed wire may have any number of shapes including straight, curved, J-tip, etc. This example may be used with any of the systems and methods disclosed herein, and it may or may not have a stent crimped to the balloon. The fixed wire catheter 5102 may be used in the main branch, or it may be used in the side branch.

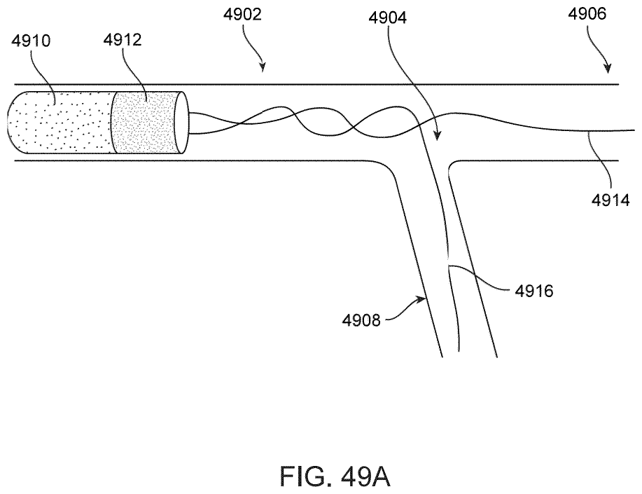

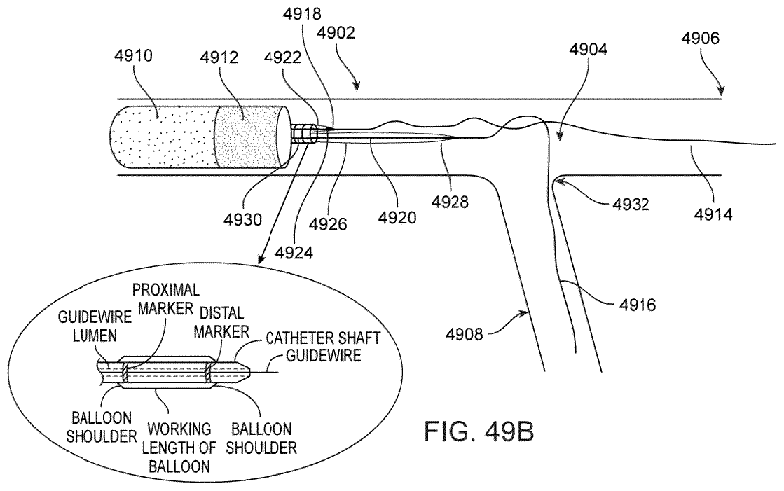

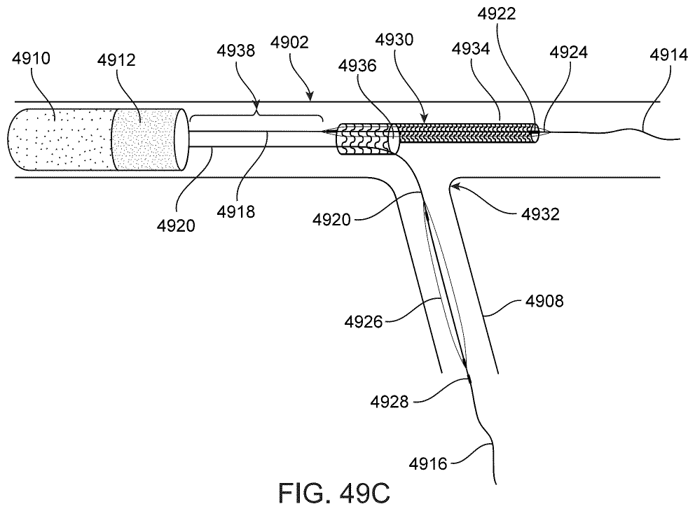

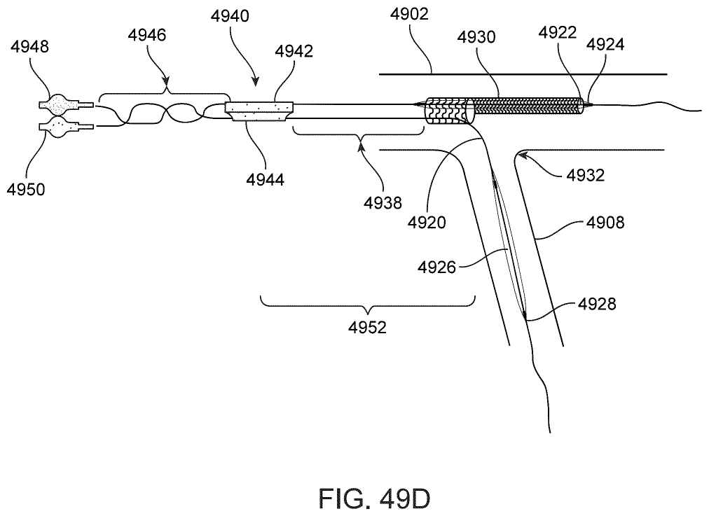

FIGS. 49A-49D include schematic views illustrating aspects of twist resolution techniques of dual catheter systems described herein. In some examples, twisting is also referred to in the art as “wire crossing”. In FIG. 49A, a blood vessel 4902 splits (or bifurcates) into a main branch 4906 and a side branch 4908 at a bifurcation 4904. In some examples, main branch is also referred to as “mother vessel” and the side branch is referred to as “daughter vessel.” The tip 4912 of a guide catheter 4910 is visible in the views. The guide catheter 4910 guides two guide wires, a main branch guide wire 4914 and a side branch guide wire 4916, through the blood vessel 4902. Beyond (distally) the bifurcation 4904, the guide wires 4914 and 4916 pass into the main branch 4906 and the side branch 4908, respectively. However, when the guide wires 4914 and 4916 are advanced through the blood vessel 4902, the guide wires 4914, 4916 can (and often do) twist around one another and can become entangled, as shown in FIG. 49A. Guide wire twisting and entanglement can present a significant problem and misdirect and prevent full deployment of stents and other devices, for example.

In FIG. 49B, a main branch catheter 4918 is deployed over the main branch guide wire 4914. The main branch catheter 4918 may include a main branch balloon 4922 and a main branch radiopaque marker 4924. A side branch catheter 4920 is deployed over the side branch guide wire 4916. The side branch catheter 4920 may include a side branch balloon 4926 and a side branch radiopaque marker 4928. One or both of the main and side branch catheters 4918 and 4920 may carry a stent 4930 for deployment at the bifurcation 4904 in accordance with any one or more of the stents, catheters, stent deployment and/or bifurcation treatment procedures described herein. Various arrangements of the components illustrated in FIGS. 49A-49B are thus possible.

For example, the arrangements and positions of the radiopaque markers in FIGS. 49A-49D are shown merely by way of example in schematic outline only. Other arrangements and positions of the radiopaque markers are possible. An example arrangement is shown in the inset view of FIG. 49B. In some examples, a radiopaque marker is positioned under a balloon, aligned with an edge of the working length of the balloon and the transition to the shoulder of the balloon. The edges of the stent are also aligned with the same position on the balloon and position of the marker.

As described above with the guide wires, when the shafts of the catheters 4918 and 4920 are advanced through the blood vessel 4902, the catheter shafts often twist around one another and can become entangled. Catheter shaft twisting and entanglement can also present a significant problem and misdirect and prevent full deployment of stents and other devices, for example. Also, in some examples, because the catheters are delivered over the guidewires, the catheters follow the twisted guidewires and so the catheters end up being twisted, in addition to their own twisting/entanglement independent of the guidewires.

However, in some examples of the dual catheter systems described herein, when the twisted regions of the guide wires 4914 and 4916 and/or the shafts of the catheters 4918 and 4920 are pushed against the carina 4932 of the bifurcation 4904, the twisting of the guide wires and/or catheters is pushed back proximally (i.e., towards the guide catheter 4910) with the result that the guide wires and catheter shafts distal of the carina 4932 become untwisted and extend straight in the main branch 4906 and the side branch 4908, as shown. In some examples, advancement of the wires or catheters against the carina at a bifurcation causes or generates a force, or reactive force, to separate the wires and/or catheters away from one another resulting in the untwisting.

In some examples, when the two catheters 4918 and 4920 are advanced over installed guidewires 4914 and 4916 distally against the carina, the twists in the catheters 4918 and 4920 get pushed back proximally. In some examples, once the guidewires 4914 and 4918 are delivered, they are not moved forward or backward. The action of the catheters 4918 and 4920 moving against the carina cause the untwisting and pushing back of the twists. This catheter twist resolution can mitigate some of the entanglement issues discussed above.

In some examples, reference is made to guide wires or catheters being advanced “against” a carina at a bifurcation to push twists in the guide wires or catheters back proximally. Unless the context is clear or implies otherwise, the use of the term “against” the carina is intended to include situations in which an untwisting guide wire and/or catheter (as the case may be) bears “directly” against a carina (i.e., while bare or uncovered for example), and also situations in which the untwisting guide wire or catheter bears “indirectly” against a carina, for example while covered by another element such as another catheter, a medical instrument, or is passing through a device, such as a stent located at the carina for example.

In FIG. 49C, the stent 4930 is advanced to arrive at the site of the bifurcation 4904. The stent 4930 can be deployed (for example, expanded) using any one of the stent deployment and/or balloon inflation techniques described further above. In some examples, the stent 4930 has a first or main branch portion 4934 and a side hole 4936. The main branch portion 4934 is carried on the main branch catheter 4918 and can be expanded by the main branch balloon 4922 (as described further above for example) to deploy, crimp, and lock the stent 4930 in place at the site of the bifurcation 4904.

In some examples, the side branch catheter 4920 passes through the side hole 4936 of the stent 4930, as shown. In the illustrated view, the side branch catheter 4920 (carrying the side branch balloon 4926 and the side branch radiopaque marker 4928) has been advanced distally past the carina 4932 of the bifurcation 4904 to enter untwisted (or at least unentangled) and in a straight manner into the side branch 4908. The side branch balloon 4926 may be inflated to expand and deploy a side branch stent (not shown in the interest of clarity) using one or more of the side branch stent deployment techniques described further above, for example.

Similarly, the main branch catheter 4918 (carrying the main branch balloon 4922 and the main branch radiopaque marker 4924) has been advanced distally past the carina 4932 of the bifurcation 4904 to enter untwisted (or at least unentangled) and in straight manner into the main branch 4906. The presence of the deployed (expanded) stent 4930 may serve to lock the stent 4930 rotationally relative to the carina 4932 and serve as a guide for the catheters 4918 and 4920. The rotationally locked stent 4930 may serve to reinforce the carina 4932 and/or assist in untwisting the catheters 4918 and 4920 and pushing twists in the catheters 4918 and 4920 back proximally. An untwisted zone may be created, as shown in the untwisted region 4938 in FIG. 49C for example. In some examples, the action of the catheters 4918 and 4920 moving against the carina initially cause creation of a twisted zone adjacent the carina. Further action of the catheters 4918 and 4920 moving against the carina cause the twisted zone to clear and push back the twists.

As the catheters advance, twists and entanglements of the catheters are pushed back proximally (safely towards the catheter operator, and away from the treatment site), and into or past the guide catheter 4910 in some examples. The pushing back of twists and entanglements is accomplished or at least assisted in some examples by the resistance offered by the carina at a treated bifurcation to part twisted guide wires and/or catheters. The pushing back of twists and entanglements is accomplished or at least assisted in some examples by a stent deployed at a treated bifurcation acting as a “locked” guide serving to untwist entangled guide wires and/or advancing catheters. The pushing back of twists and entanglements in guide wires and/or advancing catheters is accomplished or at least assisted in some examples by the carina and stent acting in concert together as when used for example in the dual mode catheter systems and methods described herein. As described above with reference to FIG. 38D, an operator may feel resistance against further advancement of the catheters because as the catheters are advanced further distally, the two catheter shafts will spread apart (and untwist) relative to one another as they are forced against the carina of the bifurcation.

With reference to FIG. 49D, the proximal pushing or passing back of twists and entanglements can be assisted by the employment of a slider or “pocket” 4940. In some examples, the pocket 4940 includes or is constituted by a hollow exchange port tube, or a capture tube, or a removable capture tube, or a zipper or snap fitting as described further above. An example pocket 4940 illustrated in FIG. 49D has two channels, for example tubular or hollow channels, to accommodate and guide in sliding fashion the shafts of the main branch catheter 4918 and the side branch catheter 4920, respectively. Other arrangements and configurations of the pocket 4940 are possible. In the illustrated view, the pocket 4940 includes a tubular main branch catheter channel 4942 and a side branch catheter channel 4944. The pocket 4940 serves to pass twists and entanglements proximally and safely into a twist collection region 4946 nearer an operator handling catheter connectors 4948 and 4950, for example. In some examples, when the twists unwrap at the distal end of the dual-catheter system, there is little to no “whiplash” experienced at the proximal end, for example nearer an operator or away from the treatment site. In other words, as the distal ends of the wires and/or catheters untwist to release twists and wire crosses, the proximal end of the wires and/or catheters rotate smoothly and consistently without a jerky or start/stop rotation.

In some examples, the pocket 4940 is interposed between the bifurcation and the guide catheter tip, in use. In some examples, the pocket 4940 is positioned in use on the catheter shafts at a pocket-to-bifurcation distance 4952 in the range 30-100 millimeters (mm). In some examples, the pocket 4940 is positioned in use on the catheter shafts at a pocket-to-bifurcation distance 4952 in the range 30-50 mm.

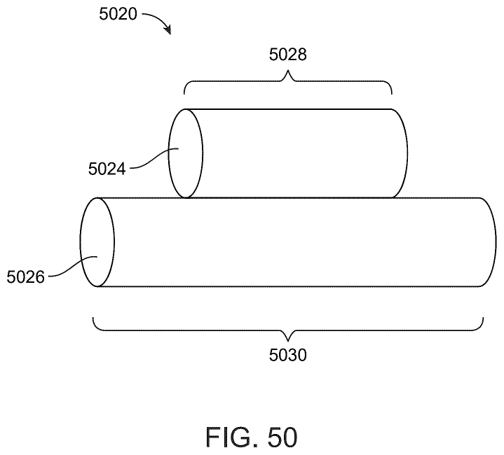

An enlarged pictorial view of an example pocket 5020 appears in FIG. 50. The pocket 5020 includes a first catheter shaft channel 5024 and a second catheter shaft channel 5026. A length 5028 of the first catheter shaft channel 5024 may be the same as, shorter, or longer than a length 5030 of the second catheter shaft channel 5026. A diameter or cross sectional area of the first catheter shaft channel 5024 may be the same as, smaller, or greater, than a diameter or cross sectional area of the second catheter shaft channel 5026.

The length 5028 of the first catheter shaft channel 5024 may be in a range 5-60 mm, or in a range 10-40 mm, or in a range 20-40 mm, or be 36 mm. The length 5030 of the second catheter shaft channel 5026 may be in a range 5-60 mm, or in a range 10-40 mm, or in a range 20-40 mm, or be 36 mm. The first catheter shaft channel 5024 may have an outer diameter in a range 0.040-0.050 mm, and an internal diameter in the range 0.035-0.045 mm. The second catheter shaft channel 5026 may have an outer diameter in a range 0.030-0.040 mm, and an internal diameter in the range 0.025-0.035 mm.

The pocket 5020 (or at least one of the catheter shaft channels 5024 and 5026) may be coextruded with a shaft of the main branch catheter or the side branch catheter, or it may be bonded or otherwise attached thereto using techniques known to those skilled in the art. The catheter shaft channels are sized to slidably receive a portion of a catheter shaft passing therethrough. In some examples, an elongate slot or slots (not shown) may extend along the entire length of the pocket 5020 and be sized such that the pocket 5020 may be snapped onto one or both catheter shafts ready for use.

Some examples of dual catheter systems include asymmetric profiles to assist with twist resolution. For example, a catheter shaft may include a non-circular portion that helps with twist resolution, and/or in proximally pushing or passing back twist and entanglements as discussed above. To this end, a catheter shaft (such as the main branch catheter and/or the side branch catheter) may have an oval cross section, at least an oval cross section in a length of the shaft close to or traversing a treated bifurcation, or a pocket, for example. Other cross-sectional shapes are possible and may be beneficial in that they reduce the overall profile of the dual catheter system. For example, reference in this regard is made to FIG. 35 hereof which shows a pear-shaped overall profile that may be adopted for dual catheter profiles to assist in twist resolution techniques. Other overall profiles for dual catheters, or a pocket, or a pocket channel are possible, for example a figure of eight configuration. A cross section of a catheter shaft, pocket, or pocket channel having a cross-sectional transverse or lateral dimension being greater in one direction than another transverse or lateral dimension in another direction (for example, orthogonal thereto) may serve to impart anti-twist resistance because of the non-circular configuration presented by such a profile. An asymmetric cross-sectional profile of a catheter shaft may present a larger bearing surface area against a carina, for example, to impart a larger “anti-twist” torque or push-back force than a catheter shaft of circular cross section, for example. In some examples, a pocket 4940 and/or shaft 5002 may include a cross-sectional profile similar to that of the snap fitting 1324 disclosed in FIG. 13C, for example. In other words, one pocket channel may be larger in diameter or sectional area than another.

Some examples also relate more generally to untwisting wires or cables at the site of a bifurcation in broader applications, such as at a bifurcation in a tunnel in underground mining or boring, or at a bifurcation in downhole logging applications, for example. Disclosed twist resolution techniques may also be used at aboveground construction sites, for example, where twisting of wires and cables at bifurcations in guides or scaffolding structures may occur. Other twist resolution applications are possible in domestic or commercial IT cabling, such as fiber optic cable installation, for example. Other twist resolution applications are possible.

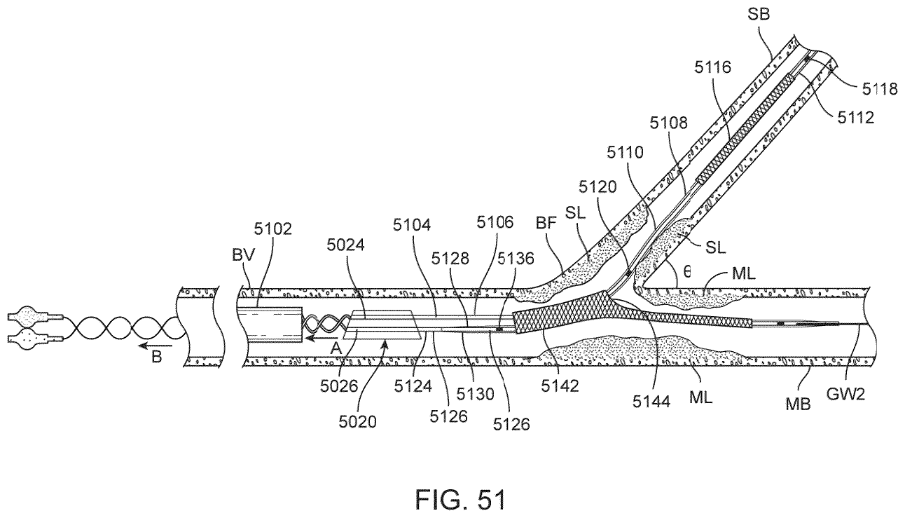

In medical device applications, FIG. 51 illustrates an example stent installed using example twist resolution techniques of a dual catheter system. In FIG. 51, both catheters 5104, 5124 are advanced distally toward a bifurcation BF until a first stent 5116 is distal to a side branch lesion SL and a second stent 5142 traverses a main branch lesion ML and a side hole 5144 is adjacent the ostium of the side branch SB. Advancement of both catheters 5104, 5124 is again performed simultaneously, although they could also be advanced independently of one another. The operator will feel resistance against further advancement of the catheters 5104, 5124 because as the catheters are advanced further distally, the two catheter shafts 5106, 5126 will spread apart relative to one another as they are forced against the carina of the bifurcation. However, a portion of the first elongate shaft 5106 is disposed under a portion of the second stent 5142; therefore the two shafts 5106, 5126 can only spread apart so far. Thus, when an operator feels resistance against further advancement of the catheter shafts 5106, 5126, the operator knows that both catheters 5104, 5124 and their associated stents and balloons are properly positioned relative to the bifurcation. Further, while the catheter shafts 5106, 5126 are being advanced and pushed against the carina of the bifurcation, twists in the catheters are pushed back proximally in the direction of arrow A (i.e., towards the fixed wire catheter 5102) with the result that the guide wires and catheter shafts distal of the carina become untwisted and extend straight into the main branch MB and side branch SB, as shown. With the help of the pocket 5150 positioned on the catheter shafts, the pushing or passing back of the twists can extend into the region of arrow B and towards the catheter operator, as shown.

In subsequent operations, the first catheter 5104 is retracted proximally relative to the second catheter 5124. Because a portion of the first catheter shaft 5106 is disposed under a portion of the second stent 5142, the first shaft 5106 is slidably retracted into side hole 5144 and the first shaft 5106 and proximal portion 5110 of balloon 5108 are slidably retracted under a portion of second stent 5142. The first catheter shaft 5106 is proximally retracted until proximal radiopaque marker 5120 lines up with proximal radiopaque marker 5136 so that a proximal end of the first stent 5116 will be aligned with the side hole 5144 in the second stent 5142. An operator may feel resistance during retraction of the first elongate shaft 5106 relative to the second elongate shaft 5126 when the ends of the stents 5116, 5142 engage one another. Stent 5142 has a distal portion crimped to balloon 5128 to prevent ejection during delivery, and a proximal portion is partially crimped thereto or uncrimped to allow catheter 5104 to slide thereunder. Ultimately, both stents 5116, 5142 are disposed adjacent their respective lesions SL, ML, and the side hole 5144 is in rough alignment with the ostium to the side branch SB and the side branch stent 5116.

The balloon 5108 is radially expanded, often with contrast medium, saline, or a combination thereof thereby radially expanding the first stent 5116 into engagement with the side branch lesion SL and the walls of the side branch. A proximal portion 5110 and a distal portion 5112 of the balloon 5108 will also expand, thus a proximal portion of the second stent 5142 will also be radially expanded. Expansion of the stents occurs simultaneously. Since a portion of balloon 5108 also passes through side hole 5144, expansion of balloon 5108 also partially expands the side hole 5144 and also aligns the side hole 5144 with the ostium of the side branch.

In a subsequent operation, the balloon 5108 is contracted, and the other balloon 5128 is radially expanded, with contrast medium, saline, or a combination thereof, thereby further radially expanding the second stent 5142. Expansion of balloon 5128 expands the proximal portion of the stent 5142 into engagement with the main branch vessel wall and main branch lesion ML, and the distal portion of the stent 5142 is also radially expanded into the main branch vessel wall as well as the main branch lesion ML. The side hole 5144 is also further aligned with the ostium of the side branch SB. Kissing balloon techniques as described further above can be used to further align and engage the stents, followed by deflation of the balloons and retraction of the catheters, as described for example further above with reference to FIGS. 38A-38M.

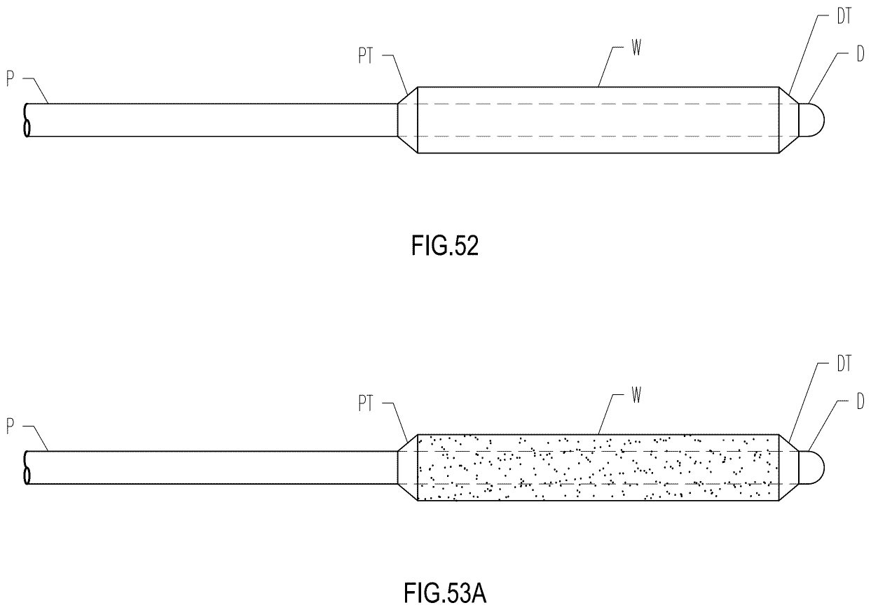

FIG. 52 shows an example of an expandable member, here a balloon coupled to an elongate shaft having proximal end P and a distal end D. The balloon has an elongate cylindrical working length W when expanded and proximal taper PT, and distal taper DT. The proximal and distal tapers provide a transition from the expanded diameter of the working length to the outer surface of the elongate shaft where the balloon is coupled to the elongate shaft. This balloon may be used on any of the delivery catheters disclosed herein, such as a mother catheter or a daughter catheter, or both mother and daughter catheter.

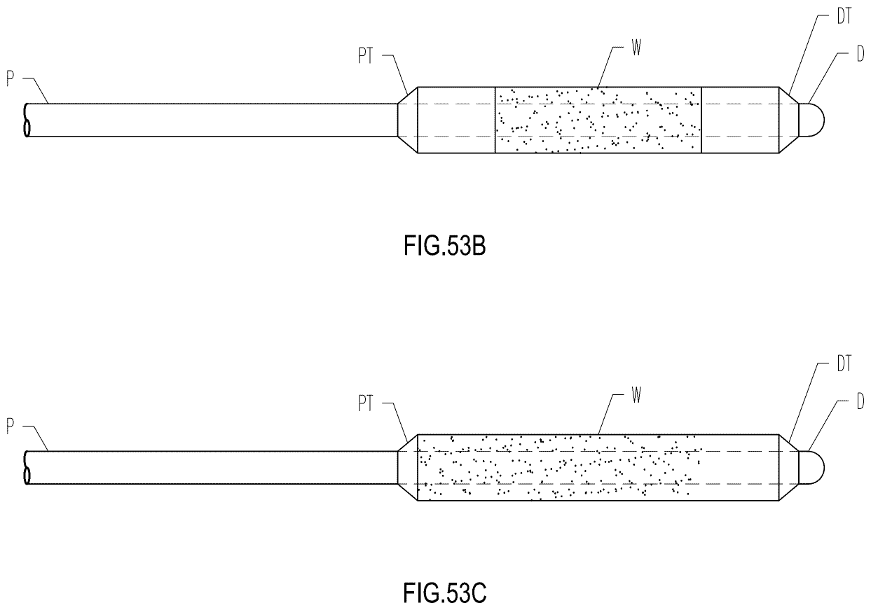

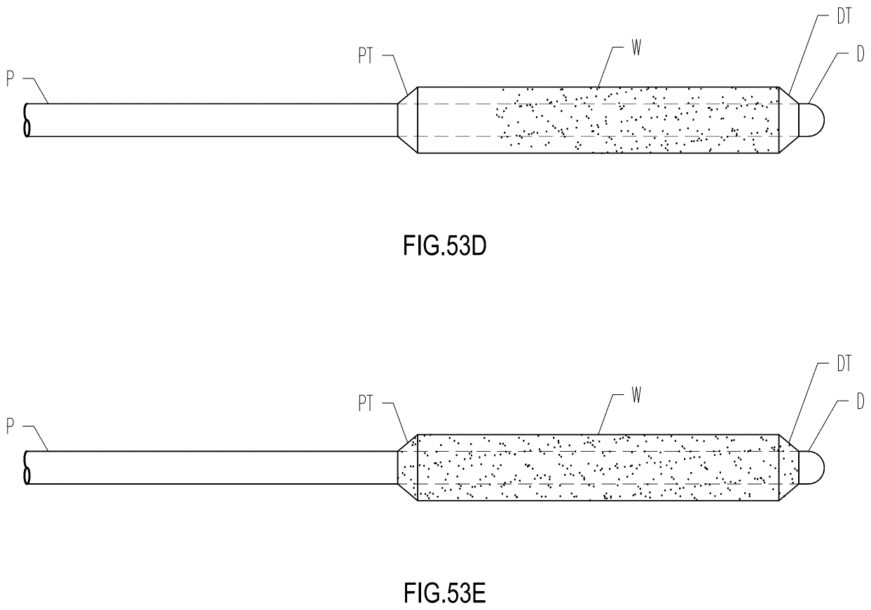

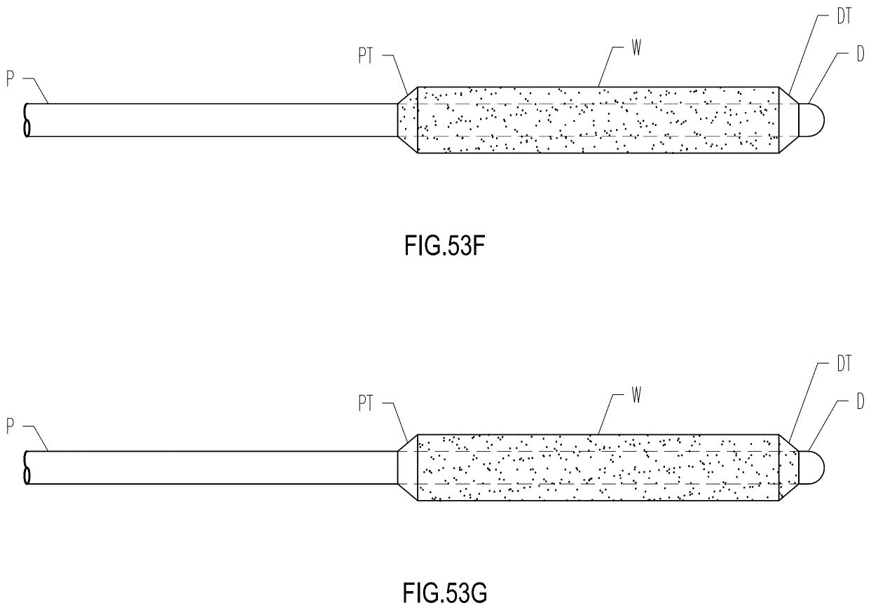

FIGS. 53A-53G illustrate various examples of how the therapeutic agent can be carried by the expandable member. These examples are not intended to be limiting, and one of skill in the art will appreciate that any coating or pattern may be used.

FIG. 53A shows the therapeutic agent or combination of therapeutic agents coated or otherwise carried on the entire working length W of the balloon while the proximal taper PT and distal taper DT remain free of any therapeutic agent.

FIG. 53B shows a similar example to FIG. 53A, except in this example the therapeutic agent or combination of therapeutic agents is disposed on the working length, but does not cover the entire working length. Here, a proximal portion of the working length and a distal portion of the working length remain free of the therapeutic agent. Other aspects of FIG. 53B are generally the same as in FIG. 53A such as the proximal taper and distal taper remaining free of any therapeutic agent.

FIG. 53C shows a similar example as FIG. 53B, except here, the working length of the balloon carries a therapeutic agent or combination of therapeutic agents from a central portion proximally all the way proximally until the proximal taper. The proximal taper remains free of a therapeutic agent. A distal portion of the balloon remains free of the therapeutic agent along with the distal taper.

FIG. 53D is similar to FIG. 53C except the coating is reversed relative to FIG. 53C. Here, the therapeutic agent or combination of therapeutic agents is disposed on a central portion of the balloon and distal portion of the balloon all the way to the distal taper, while the distal taper is free of a therapeutic agent. A proximal portion of the working length of the balloon remains free of the therapeutic agent along with the proximal taper.

FIG. 53E illustrates an example where the entire expandable member carries a therapeutic agent or combination of therapeutic agents. Here, the therapeutic agent or combination of therapeutic agents is disposed along the entire working length of the balloon as well as on both the proximal taper and distal taper.

FIG. 53F shows a variation of FIG. 53E. Here, the therapeutic agent or combination of therapeutic agents coats the entire proximal taper and working length of the balloon and only the distal taper remains free of the therapeutic agent.

FIG. 53G is similar to FIG. 53F, except the coating is reversed relative to FIG. 53F. Here, the therapeutic agent or combination of therapeutic agents covers the entire working length of the balloon and the distal taper, while the proximal taper remains free of the therapeutic agent.

One of skill in the art will appreciate that any permutation or combination of the therapeutic agent patterns described above may be used in any of the delivery systems disclosed herein. Additionally, when referring to a therapeutic agent or combination of therapeutic agents that is only partially disposed on a section of the balloon, one of skill in the art will appreciate that the proportion of the balloon region that is covered or uncovered may be varied to any degree desired. Therefore, for example when referring to a working length of the balloon being only partially covered with a therapeutic agent, the working length may be covered with any percentage of surface area less than 100%. Similarly, when referring to a portion of the balloon that remains free of the therapeutic agent, all, or any percentage less than 100% of the surface area in that region may remain free of the therapeutic agent. In some examples, the balloon surface area may be covered 25% to 75% with a therapeutic agent.

In any of the examples of balloons that carry a therapeutic agent or combination of therapeutic agents, the therapeutic agent may be coated or otherwise coupled to the balloon. Other techniques known in the art may be used to couple the therapeutic agent to the balloon, e.g. spraying, dip coating, electrostatic coating, embedding the therapeutic agent into the balloon material, etc.

Additionally, radiopaque markers may be disposed on the elongate shaft carrying the balloon in order to indicate the proximal and distal edges of the therapeutic agent. For example, when the drug is disposed along the entire working length of the balloon, a proximal marker may be disposed at the proximal-most end of the working length before the proximal taper, and a distal marker may be disposed at the distal-most end of the working length just before the distal taper. In the example where the drug is only along a portion of the working length of the balloon, the marker may be disposed at the proximal edge of the therapeutic coating and a distal marker may be disposed at the distal edge of the therapeutic coating. This helps the operator visualize where the drug coating is so that the drug coating region may be aligned with the treatment area in the vessel to ensure that the therapeutic agent is effectively eluted to the desired target treatment tissue.

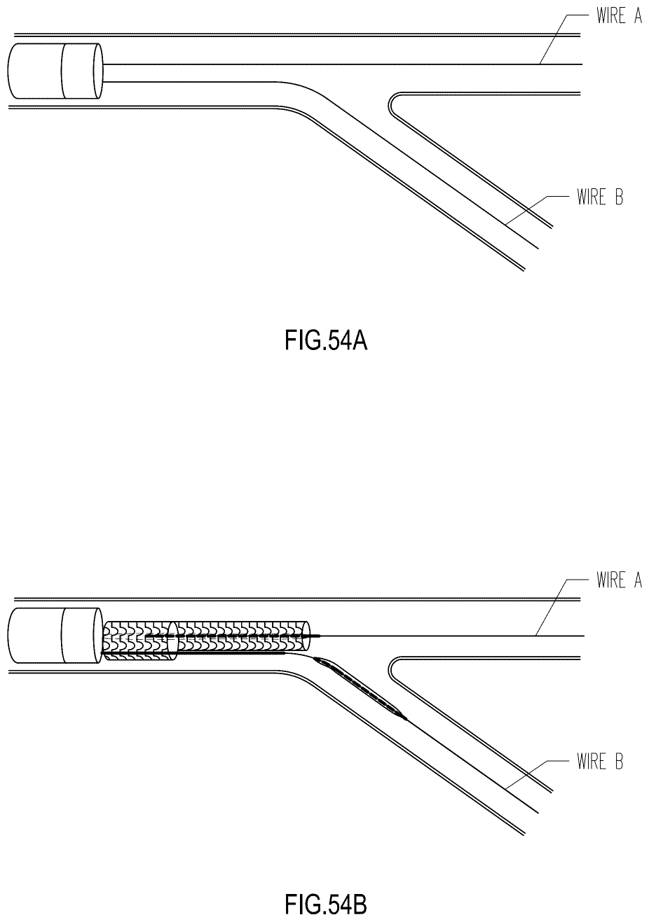

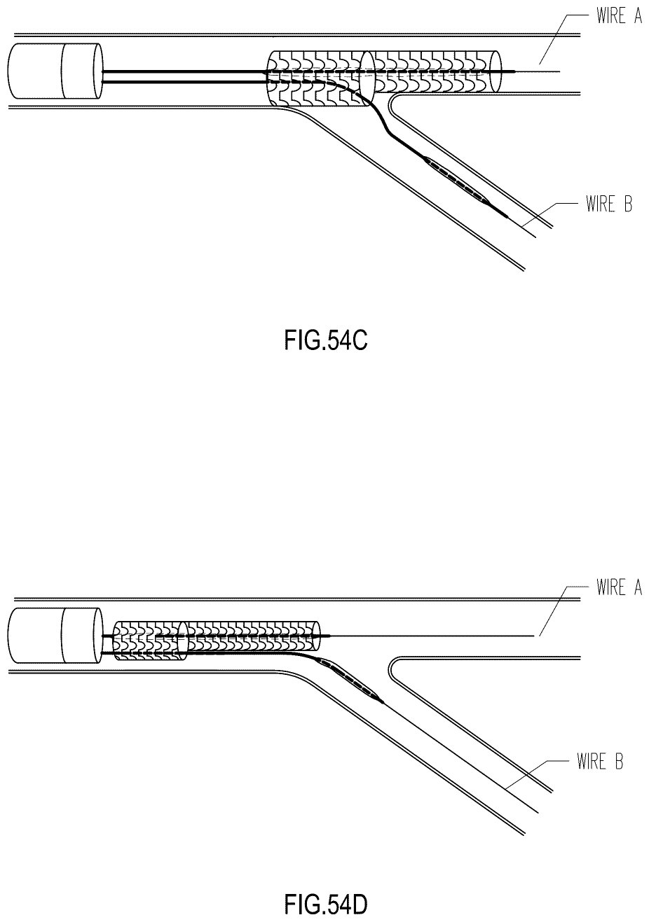

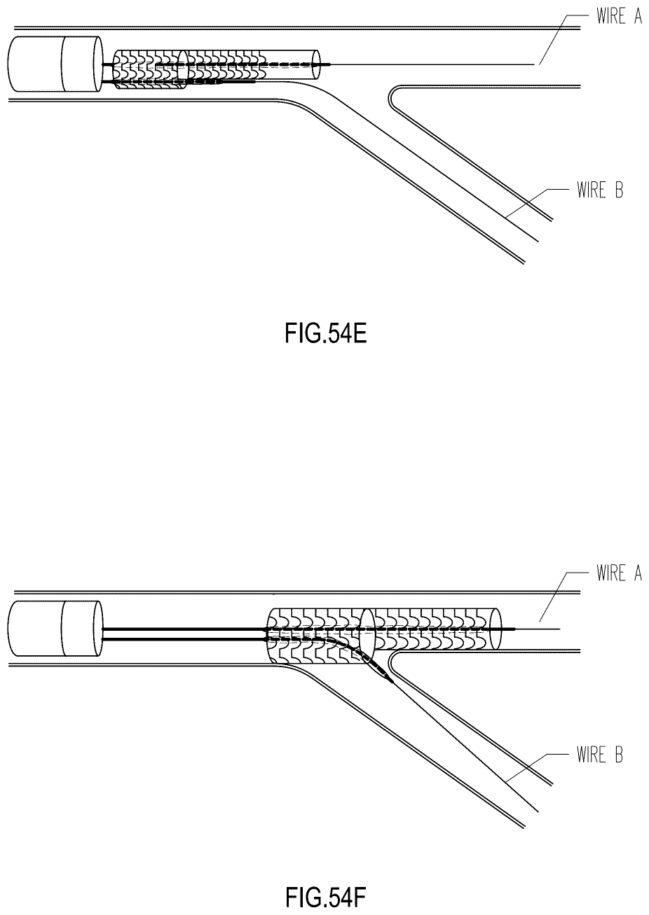

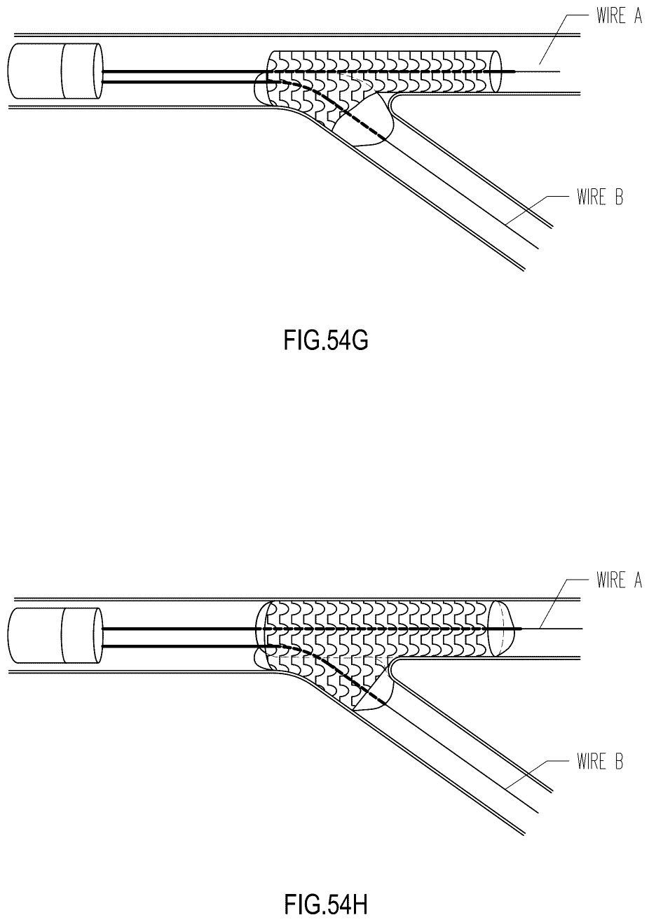

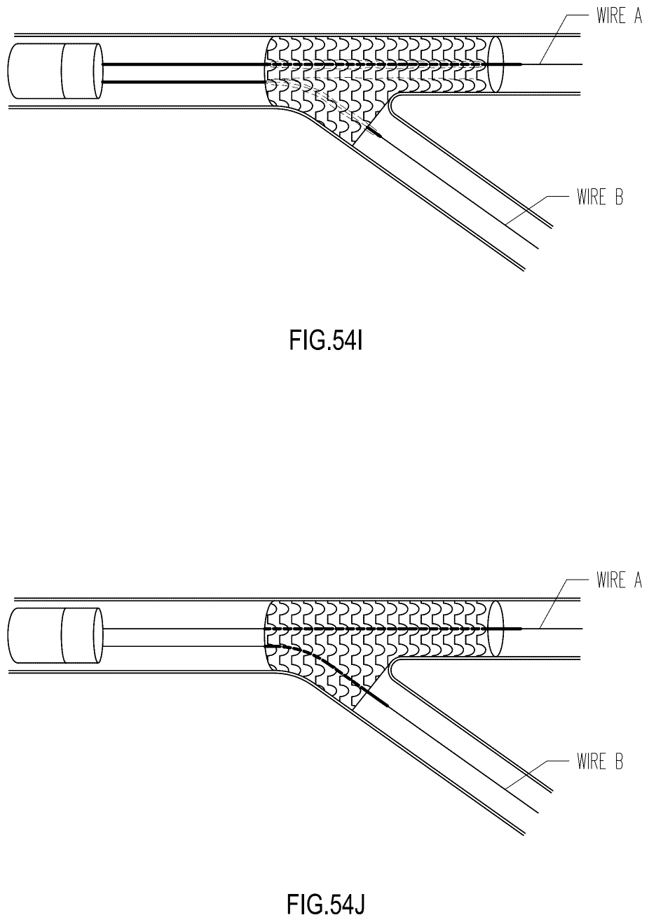

FIGS. 54A-54K illustrate an example of treating a bifurcated vessel with an expandable member carrying a therapeutic agent or combination of therapeutic agents such as any of those disclosed herein. Any of the delivery systems carrying a therapeutic agent according to any of the examples disclosed herein may be used in this example method. Additionally, any of the features described in this specification from any of the devices, systems and methods may be used in combination with or substituted for any of the features disclosed in this example of a treatment method.

FIG. 54A shows a bifurcated vessel having a mother vessel (the vessel extending horizontally past the bifurcation) and a daughter vessel (the vessel angulated after the bifurcation). The mother vessel may be referred to as the main branch and the daughter vessel may be referred to as the side branch. In the situation where the side branch has a stenotic lesion, it may be advantageous to treat the side branch with a drug coated balloon, where the drug (also referred to herein as a therapeutic agent) pharmacologically reduces or eliminates the stenotic lesion as opposed to the simple mechanical reduction in stenosis provided by POBA (plain old balloon angioplasty), and the subsequent mechanical support provided by a stent. Guidewires are introduced using techniques known in the art (e.g. surgical cut down or percutaneous introduction such as with the Seldinger technique) so that one guidewire is inserted through a guide catheter into the main branch and extending distally past the bifurcation while a second guidewire is also inserted through the guide catheter distally past the bifurcation into the side branch. Examples of guidewires may be 0.014″ guidewires, but this is not intended to be limiting.

FIG. 54B shows a stent delivery system such as any of those disclosed herein being advanced over both the main branch guidewire and the side branch guidewire. The mother catheter in this example includes a stent disposed over the mother balloon, and the daughter catheter only has a drug coated balloon without a stent. The daughter balloon is axially offset from the mother balloon such that the daughter balloon leads the mother balloon (e.g. the daughter balloon is distal of the mother balloon) so that the profile of the delivery device is smaller than if the mother balloon and daughter balloon where stacked on top of one another. The delivery catheter is advanced over both guidewires and through the guide catheter toward the bifurcation. The side branch balloon is advanced into the side branch. In this example, the daughter catheter does not have a stent. In some examples, a stent may be disposed over the daughter catheter while in some examples the daughter catheter never includes a stent.

FIG. 54C shows that the delivery system is advanced distally toward the bifurcation until the stent and balloons are adjacent the carina of the bifurcation. In some examples, there is no contact between the delivery system and the carina, while in other examples there may be contact. The side hole in the mother stent is also oriented so that it is adjacent the ostium to the side branch, and the distal end of the mother stent may be distal of the bifurcation depending on the length of the mother stent. The proximal end of the mother stent may be proximal of the ostium to the side branch. In some examples, the daughter balloon is expanded directly into the side branch tissue so that the drug elutes directly into the tissue without the stent obstructing contact.

FIG. 54D illustrates that the operator may proximally retract the entire delivery system (e.g. both the mother catheter and the daughter catheter) toward the guide catheter. In some examples, the delivery system may be retracted at least 1 cm so that the distal tip of the mother catheter is just proximal to the side branch vessel. The daughter balloon may be partially disposed in the side branch and partially disposed in the main vessel. While FIG. 54D shows the daughter balloon disposed in the side branch and the mother stent in the main branch, the system may be delivered such that the daughter balloon is disposed in the main branch and the mother stent is disposed partially in the side branch and partially in the main branch, as will be illustrated later. Thus, treatment of the side branch with the drug coating on the daughter balloon is not limited. In some cases, the daughter balloon may be advanced into the side branch and the mother stent delivered to the main branch when the bifurcation angle is less than 60-70 degrees as previously discussed. In other examples, when the bifurcation angle is greater than 60-70 degrees, the mother stent may be advanced into the side branch and the daughter catheter delivered into the mother vessel.

FIG. 54E illustrates proximal retraction of the daughter catheter into the side hole of the mother stent. Here, the proximal radiopaque marker on the daughter catheter may be axially aligned with the proximal radiopaque marker of the mother catheter. A proximal portion of the daughter balloon may be disposed under a proximal portion of the mother stent, and a distal portion of the daughter balloon may be disposed alongside an outer surface of a distal portion of the mother stent. This portion of the daughter balloon may include the drug coating since in some cases it may be beneficial not to retract the drug coated portion of the daughter balloon through the side hole of the mother stent and into the mother stent since the mother stent can abrade the drug coating and damage the coating or remove it from the daughter balloon. Once the radiopaque markers are aligned, the axial position of both the mother and daughter catheters may be maintained relative to one another.

FIG. 54F shows advancement of both the mother catheter and the daughter catheter distally toward the bifurcation. Both catheters may be advanced simultaneously and their axial positions relative to one another may be maintained during the distal advancement. In other examples, the mother catheter and daughter catheter may be advanced independently of one another. Distal advancement is continued until a tension is felt by the operator when the two catheters reach the carina. In some examples, no contact between the carina and the mother or daughter catheters is made, while in some examples there could be contact between the carina and either one or both of the mother and daughter catheters. Here, the daughter catheter is advanced into the side branch such that the drug coated portion of the daughter balloon is at least partially disposed in the side branch, while the side hole of the mother stent is aligned with the ostium of the side branch, and the distal portion of the mother stent that is distal of the side hole is disposed in a distal portion of the main branch distal of the bifurcation. The proximal portion of the mother stent proximal of the side hole is disposed in the main branch of the vessel proximal of the ostium to the side branch.

FIG. 54G shows inflation of the drug coated balloon in the side branch while the mother catheter balloon remains unexpanded. The expanded daughter balloon abuts the walls of the side branch from the ostium distally into the side branch. The proximal portion of the daughter balloon also expands under the mother stent, expanding the proximal portion of the mother stent that is proximal of the side hole. The side hole is also expanded so that the side hole aligns and conforms with the ostium to the side branch vessel. Inflation times and pressure may be varied according to operator preference, for example inflation time may be for 15 to 30 seconds. In another example, inflation pressure may not exceed 8 atmospheres of pressure. Thus, the mechanical pressure applied to the stenotic side branch lesion will help reduce the stenosis by compressing the lesion into the vessel walls and the therapeutic agent will elute from the balloon into the adjacent tissue and also help prevent restenosis.

FIG. 54H illustrates kissing balloons where the mother balloon is also inflated while the daughter balloon remains inflated. This ensures even expansion of the entire mother stent so that it conforms to the mother vessel and the ostium of the side branch and also ensures even compression of the lesion to avoid plaque shifting. Again, inflation times and pressures for the mother balloon may be varied according to operator preference. In one example, inflation time is 15 to 30 seconds, and inflation pressure does not exceed 18 atmospheres of pressure. In another example, the daughter balloon may be inflated to higher than the initial 8 atm. of pressure. Other inflation times and pressures may be used.

FIG. 54I shows deflation of both the mother balloon and the daughter balloon after adequate time for the drug to elute from the daughter balloon into the side branch vessel. The drug helps to prevent restenosis after the procedure. Deflation of the balloons may be performed simultaneously or one after the other.

FIG. 54J shows proximal retraction of the delivery system through the guide catheter and out the patient. Proximal retraction may be performed simultaneously or one catheter after the other. In some examples, the daughter catheter may be proximally retracted into the guide catheter followed by proximally retracting the mother catheter into the guide catheter. This helps ensure that both catheters can be easily removed from the patient, and then both the guide catheter and both mother and daughter catheters can be removed from the patient simultaneously as one unit. Of course, this is not intended to be limiting and the operator may retract and remove the balloon catheters and guide catheters in any order or any desired manner.

FIG. 54K shows the final step where both guidewires are retracted proximally and removed from the patient either through the guide catheter if it is still in the patient, or by proximally retracting both guide wires through the vasculature if the guide catheter has already been removed. This leaves only the stent in the main branch and ostium of the side branch with the drug eluted into the ostium of the side branch and the walls of the side branch.

FIGS. 54A-54K illustrate the use of the mother-daughter catheter system disclosed herein to deliver and elute a therapeutic agent to the side branch vessel and ostium of the side branch while a mother stent is delivered to the main branch. This is not intended to be limiting. One of skill in the art will appreciate that the mother-daughter system can be rotated so that the mother catheter is delivered to the side branch, and the daughter catheter is delivered to the main branch. This may be desirable when the bifurcation angle is greater than 60-70 degrees. Thus, the stent will be delivered to the side branch with a portion also disposed in the main branch and the side hole facing the distal portion of the main branch vessel, and the drug will be delivered to the main branch vessel and the ostium to the side branch. The method in this situation is similar to the method described above in FIGS. 39A-39M except that there is no second stent on the daughter balloon, and instead a therapeutic agent is disposed on the daughter balloon.

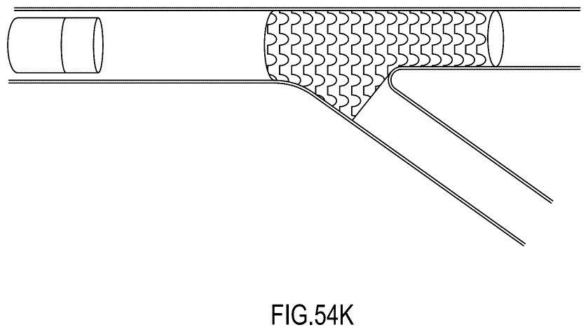

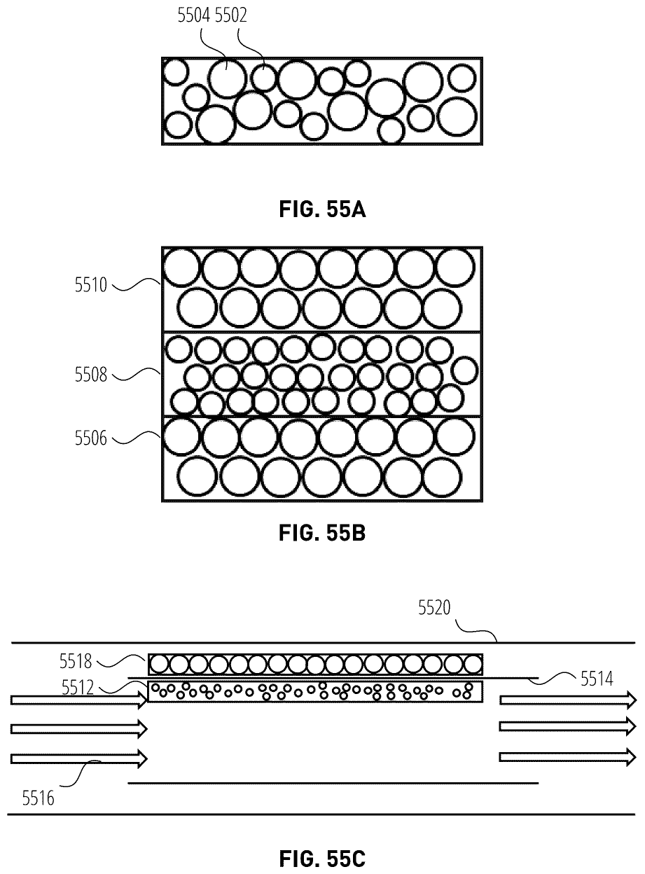

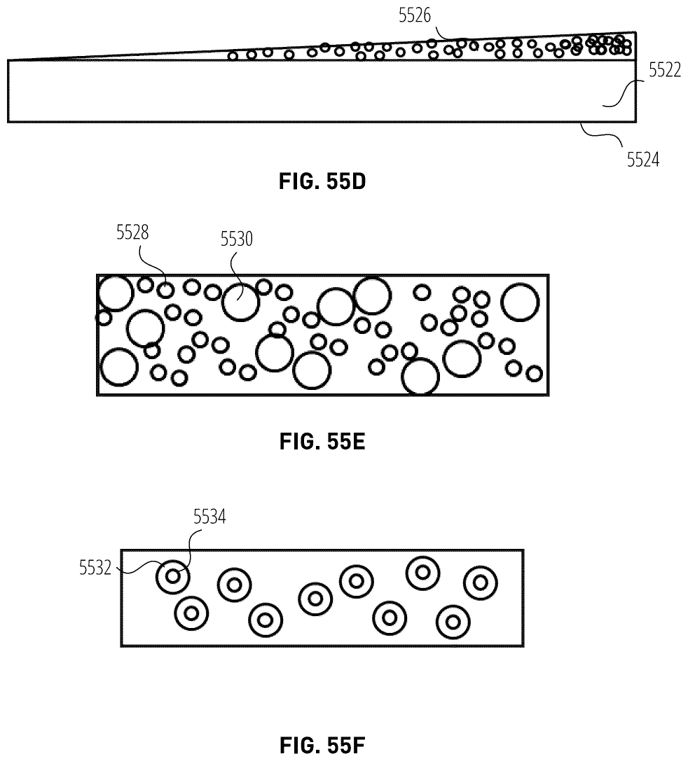

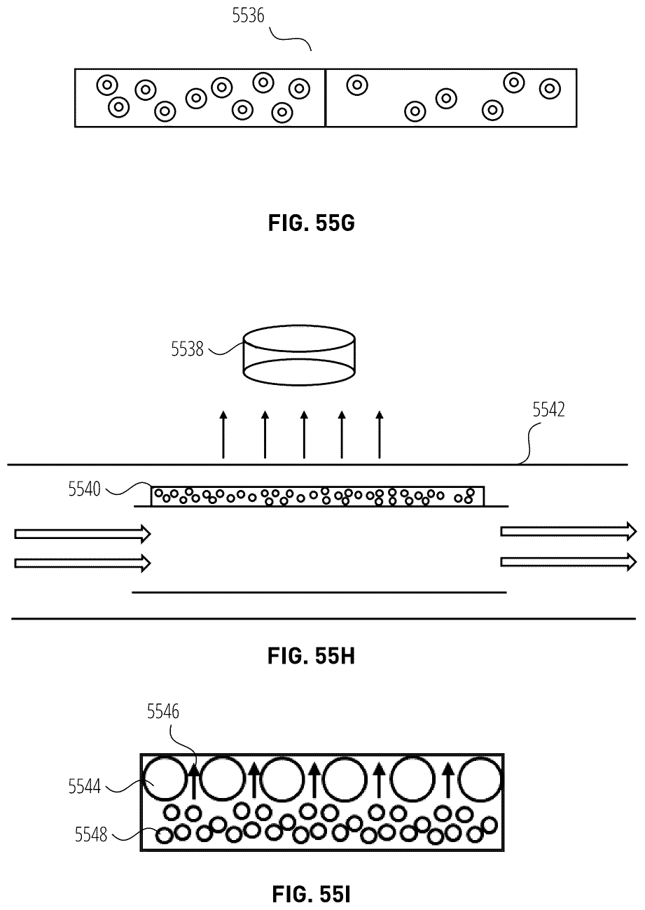

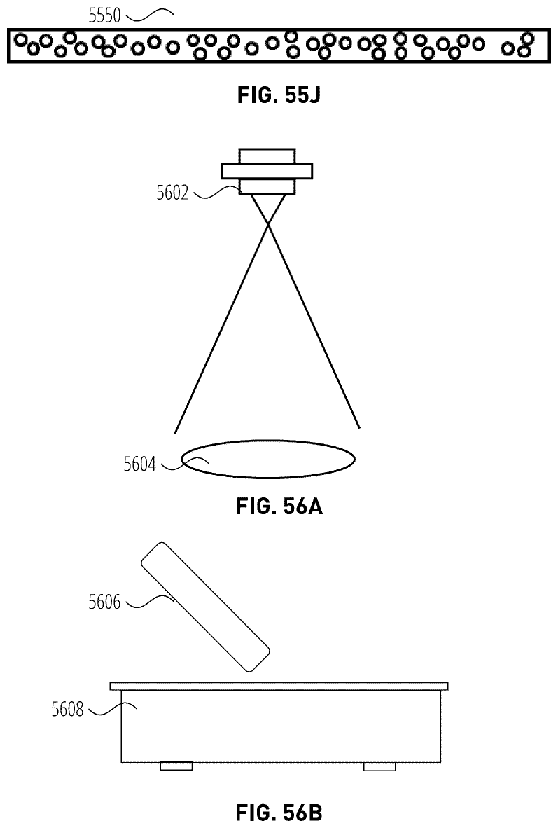

Referring now to FIG. 55A, in some examples, the therapeutic agent may be incorporated into or applied onto the stent, the balloon, or both, in a variety of configurations specifically designed to optimize drug delivery characteristics. For example, the coating comprises a single homogeneous layer in which the therapeutic agent (5502) is intimately mixed within a polymer carrier (5504) to form a uniform matrix. In some examples, the coating may include multiple distinct layers arranged in a stratified manner. As shown in FIG. 55B, a base polymer layer (5506) can be first applied directly onto the surface of the balloon or stent to enhance adhesion, followed by an intermediate layer (5508) containing the therapeutic agent dispersed within a polymer binder, and optionally capped by a topcoat polymer (5510) layer that serves to regulate the release rate or protect the active agent during catheter advancement and deployment. In some examples, as shown in FIG. 55C, the coating may comprise a bi-facial configuration where different therapeutic agents or concentrations are applied to different areas or surfaces of the same device, such that the luminal surface (5512) of a stent (5514) may carry a first therapeutic agent optimized for contact with blood flow (5516), while the abluminal surface (5518) carries a second therapeutic agent designed for penetration of vessel (5520). In some examples, a gradient coating, in which the concentration of the drug varies continuously or stepwise along the axial length or circumference of the device, may be used. As shown in FIG. 55D, by way of example, such a gradient may be configured such that the drug concentration (5526) is higher toward the proximal or distal regions (5522) of the balloon or stent (5524), such as adjacent to an ostium or bifurcation region, to achieve a localized therapeutic effect. In some examples, the coating includes thermally-responsive elements that alter drug release characteristics based on body temperature fluctuations, incorporating phase-change materials or temperature-sensitive polymers that modify permeability or dissolution rates at physiological temperatures.

In some examples, the coating includes microparticles or nanoparticles of the therapeutic agent dispersed within a polymeric matrix. As shown in FIG. 55E, the microparticles may have a defined size distribution selected to modulate the release kinetics, for instance by including a mixture of rapidly dissolving particles (5528) and slower-dissolving particles (5530) to achieve both an initial burst release and a sustained release over a predetermined period. As shown in FIG. 55F, in some examples, discrete microspheres comprising biodegradable polymers such as polylactic-co-glycolic acid may be incorporated within or adhered onto the device surface, each microsphere (5532) encapsulating the therapeutic agent (5534) in a controlled-release reservoir. As shown in FIG. 55G, in some examples, the coating may be configured as a multi-reservoir system comprising discrete compartments (5536), each containing different therapeutic agents with independent release profiles, where these compartments may be formed through micro-molding, photolithography, or selective polymer deposition techniques. As shown in FIG. 55H, in some examples, magnetically-responsive elements such as magnetic nanoparticles (5540) may be incorporated that can be activated by external magnetic fields (5538) to trigger drug release, enhance tissue (5542) penetration, or provide targeted heating for thermally-triggered drug delivery.

In some examples, a hydrogel-based coating, which expands upon hydration when the device contacts body fluids, may be used, thereby facilitating diffusion of the therapeutic agent into the adjacent vessel wall. As shown in FIG. 55I, in some examples, the coating (5544) may be porous, comprising a network of interconnected voids (5546) or channels that enable sustained diffusion of the therapeutic agent (5548) over time. In some examples, the coating may exhibit hierarchical structuring at multiple length scales, combining macro-scale patterns (such as helical or circumferential bands) with micro-scale features (such as surface roughness or porosity) and/or nano-scale elements (such as nanoparticle inclusions or molecular organization). In some examples, stimuli-responsive coatings may be employed that alter their properties in response to specific biological conditions such as pH changes, enzyme activity, mechanical stress, or the presence of specific biomarkers associated with disease progression or healing.

As shown in FIG. 55J, in some examples, the coating (5550) is substantially free of polymer and comprises a crystalline or amorphous deposit of the therapeutic agent alone, optionally configured to dissolve rapidly upon exposure to blood or tissue fluids to deliver a bolus dose. In some examples, molecular self-assembly processes may be employed, where therapeutic agents are incorporated into self-organizing structures such as micelles, vesicles, or liquid crystalline phases that spontaneously form ordered coatings upon contact with the device surface.

In some examples, the total drug load on the coated surface may be adjusted across a broad range, for example, from about 0.1 micrograms per square millimeter to about 10 micrograms per square millimeter, depending upon the desired pharmacologic profile. In some examples, the ratio of therapeutic agent to polymer may range from approximately 0.1:1 to 10:1 by weight, and may be further modulated by including excipients such as plasticizers, surfactants, stabilizers, antioxidants, solubilizers, or pore-forming agents to fine-tune the mechanical properties, adhesion, and elution characteristics. In some examples, the coating process may include in-line monitoring using spectroscopic techniques such as Raman spectroscopy, infrared spectroscopy, or fluorescence detection to ensure consistent coating thickness, drug loading, and spatial distribution during the manufacturing process.

In some examples, excipients may be used to carry the therapeutic agent and enhance the delivery, stability, bioavailability, and release characteristics of the therapeutic formulation. Excipients serve multiple functions including acting as carriers, stabilizers, solubilizers, penetration enhancers, release modifiers, and processing aids that optimize the therapeutic coating performance. Some examples of excipients may include carriers, antioxidants, nanoparticles, bio-adhesives, surfactants, plasticizers, pore-forming agents, stabilizers, and other functional additives that enhance the therapeutic delivery system. In some examples, the excipients may be selected to provide specific release kinetics, improve drug solubility, enhance tissue penetration, prevent drug degradation, or modify the mechanical properties of the coating. In some examples, multiple excipients may be combined to achieve synergistic effects and optimize the overall therapeutic delivery performance.

In some examples, carriers may be used to facilitate drug loading, provide controlled release characteristics, and enhance the mechanical properties of the therapeutic coating. Examples of carriers include Shellac, Urea, Iopromide, Butyryl Trihexyl Citrate, Acetyl Tributyl Citrate, Dextran, Dimethyl Sulfoxide, Polysorbate, and Sorbitol. In some examples, additional carriers may include polyvinylpyrrolidone (PVP), hydroxypropyl methylcellulose (HPMC), hydroxypropyl cellulose (HPC), methyl cellulose, ethyl cellulose, carboxymethyl cellulose, hydroxyethyl cellulose, chitosan, alginate, pectin, gellan gum, xanthan gum, guar gum, and other cellulosic and non-cellulosic polymers. In some examples, lipid-based carriers such as phosphatidylcholine, phosphatidylserine, sphingomyelin, cholesterol, cholesteryl esters, triglycerides, diglycerides, monoglycerides, fatty acids, fatty acid esters, and waxes may be used. In some examples, protein-based carriers such as albumin, gelatin, collagen, fibrin, casein, and other biocompatible proteins may be incorporated. In some examples, cyclodextrins including alpha-cyclodextrin, beta-cyclodextrin, gamma-cyclodextrin, and their hydroxypropyl, methyl, and sulfobutyl derivatives may be used to enhance drug solubility and stability. In some examples, polyethylene glycol (PEG) of various molecular weights, polypropylene glycol, polyethylene oxide, poloxamers, and other hydrophilic polymers may serve as carriers.

In some examples, antioxidants may be incorporated to prevent oxidative degradation of the therapeutic agent during storage and after deployment, ensuring maintained potency and therapeutic efficacy. Examples of antioxidants include Butylated Hydroxyl Toluene (BHT), butylated hydroxyanisole (BHA), alpha-tocopherol (vitamin E), tocopherol acetate, ascorbic acid (vitamin C), ascorbyl palmitate, sodium ascorbate, citric acid, tartaric acid, malic acid, and other organic acids. In some examples, additional antioxidants may include propyl gallate, octyl gallate, dodecyl gallate, nordihydroguaiaretic acid (NDGA), 2,6-ditertiary-butyl-4-methylphenol, tertiary butylhydroquinone (TBHQ), and other phenolic antioxidants. In some examples, chelating agents such as ethylenediaminetetraacetic acid (EDTA), ethylene glycol tetraacetic acid (EGTA), diethylenetriaminepentaacetic acid (DTPA), and deferasirox may be used to prevent metal-catalyzed oxidation. In some examples, reducing agents such as sodium metabisulfite, sodium bisulfite, sodium sulfite, cysteine, methionine, and glutathione may be included. In some examples, radical scavengers such as mannitol, sorbitol, histidine, tryptophan, and other amino acids with antioxidant properties may be incorporated.

In some examples, nanoparticles may be used to encapsulate therapeutic agents, provide controlled release, enhance tissue penetration, and improve bioavailability through size-dependent cellular uptake mechanisms. Examples of nanoparticle materials include Polyester, Poly Lactic-co-glycolic acid (PLGA), polylactic acid (PLA), polyglycolic acid (PGA), polycaprolactone (PCL), and other biodegradable polymers. In some examples, additional nanoparticle materials may include poly(lactic-co-glycolic-co-caprolactone) copolymers, polyhydroxybutyrate, polyhydroxyvalerate, polydioxanone, polytrimethylene carbonate, and other aliphatic polyesters. In some examples, lipid-based nanoparticles such as liposomes, solid lipid nanoparticles (SLNs), nanostructured lipid carriers (NLCs), lipid nanocapsules, and microemulsions may be used. In some examples, inorganic nanoparticles such as silica nanoparticles, calcium phosphate nanoparticles, hydroxyapatite nanoparticles, iron oxide nanoparticles, gold nanoparticles, silver nanoparticles, and titanium dioxide nanoparticles may be incorporated. In some examples, polymeric micelles formed from block copolymers such as PEG-PLA, PEG-PLGA, PEG-PCL, and other amphiphilic block copolymers may be used. In some examples, dendrimers including PAMAM (polyamidoamine), PPI (polypropylenimine), and other hyperbranched polymers may serve as nanocarriers. In some examples, carbon-based nanomaterials such as carbon nanotubes, graphene oxide, and fullerenes may be used as drug carriers.

In some examples, bio-adhesives may be used to enhance the adhesion of the therapeutic coating to biological tissues, improve drug retention at the target site, and provide controlled release characteristics through bioadhesive interactions. Examples of bio-adhesives include Poly-L-lactic acid (PLLA), Poly-d,l-lactic Acid (PDLLA), Polyester Amide (PEA), Polyethylene Glycol, Poly Glycolic Acid, Polyethyleneimine (PEI), and Amino Acids such as lysine, arginine, and histidine. In some examples, additional bio-adhesive materials may include mucoadhesive polymers such as carbopol, polycarbophil, sodium carboxymethylcellulose, hyaluronic acid, chitosan, alginate, pectin, and their derivatives. In some examples, thiolated polymers (thiomers) such as thiolated chitosan, thiolated poly(acrylic acid), and thiolated cellulose derivatives may be used to form disulfide bonds with cysteine-rich domains in biological tissues. In some examples, lectin-based adhesives, fibrin-based adhesives, and other protein-based bioadhesive systems may be incorporated. In some examples, synthetic bio-adhesives such as poly(acrylic acid), poly(methacrylic acid), polyvinyl alcohol, polyvinylpyrrolidone, and their copolymers may be used. In some examples, naturally-derived bio-adhesives such as gellan gum, agar, carrageenan, fucoidan, and other marine-derived polysaccharides may be incorporated. In some examples, cell-penetrating peptides, tissue-specific targeting peptides, and other bioactive peptides may be used to enhance cellular adhesion and uptake.

In some examples, surfactants may be incorporated to improve drug solubility, enhance wetting characteristics, facilitate coating uniformity, and modify surface properties of the therapeutic formulation. Examples of surfactants include polysorbates (Tween 20, 40, 60, 80), sorbitan esters (Span 20, 40, 60, 80), poloxamers (Pluronic F68, F108, F127), sodium lauryl sulfate, sodium dodecyl sulfate, and cetyltrimethylammonium bromide. In some examples, additional surfactants may include polyoxyethylene stearates, polyoxyethylene castor oil derivatives (Cremophor EL, RH40), vitamin E polyethylene glycol succinate (TPGS), poloxamine, polysorbate derivatives, and other non-ionic surfactants. In some examples, ionic surfactants such as benzalkonium chloride, cetylpyridinium chloride, sodium stearate, and potassium oleate may be used. In some examples, amphoteric surfactants such as lecithin, phosphatidylcholine, and cocamidopropyl betaine may be incorporated. In some examples, biosurfactants such as rhamnolipids, surfactin, and other naturally-derived surface-active agents may be used.

In some examples, plasticizers may be added to modify the mechanical properties of the coating, enhance flexibility, improve processability, and control drug release rates through alterations in polymer chain mobility. Examples of plasticizers include dibutyl phthalate, diethyl phthalate, dimethyl phthalate, triethyl citrate, tributyl citrate, acetyl triethyl citrate, glycerol, propylene glycol, polyethylene glycol 200-600, and diethylene glycol monoethyl ether. In some examples, additional plasticizers may include castor oil, mineral oil, oleic acid, stearic acid, palmitic acid, isopropyl myristate, dibutyl sebacate, dioctyl adipate, and other fatty acid esters. In some examples, sugar-based plasticizers such as sorbitol, mannitol, xylitol, maltitol, and other polyols may be used. In some examples, polymeric plasticizers such as low molecular weight PEG, polypropylene glycol, and polyethylene glycol dimethyl ether may be incorporated.

In some examples, pore-forming agents may be included to create controlled porosity networks within the coating matrix, facilitating drug diffusion and enabling tunable release kinetics. Examples of pore-forming agents include sodium chloride, potassium chloride, sucrose, lactose, mannitol, sorbitol, polyethylene glycol, polyvinyl alcohol, and hydroxypropyl methylcellulose. In some examples, additional pore-forming agents may include ammonium carbonate, ammonium bicarbonate, sodium bicarbonate, calcium carbonate, magnesium carbonate, and other salts that can be leached out or dissolved to create porosity. In some examples, volatile pore-forming agents such as camphor, menthol, thymol, and other sublimable compounds may be used. In some examples, polymeric pore-formers such as polyvinylpyrrolidone, polyethylene oxide, and water-soluble cellulose derivatives may be incorporated and subsequently extracted.

In some examples, penetration enhancers may be used to facilitate drug transport across biological barriers, improve tissue penetration, and enhance therapeutic agent uptake at the target site. Examples of penetration enhancers include dimethyl sulfoxide (DMSO), propylene glycol, isopropyl alcohol, oleic acid, linoleic acid, azone (1-dodecylazacycloheptan-2-one), transcutol, and various fatty acids and their esters. In some examples, additional penetration enhancers may include terpenes such as menthol, limonene, eucalyptol, and carvone. In some examples, surfactant-based enhancers such as sodium lauryl sulfate, polysorbate 80, and Brij series may be used. In some examples, enzyme inhibitors such as aprotinin, leupeptin, and other protease inhibitors may be included to prevent drug degradation during penetration.

In some examples, stabilizers may be incorporated to maintain drug stability, prevent degradation, and preserve therapeutic efficacy during storage and deployment. Examples of stabilizers include human serum albumin, gelatin, sucrose, trehalose, lactose, dextran, mannitol, glycine, histidine, and arginine. In some examples, additional stabilizers may include cyclodextrins, polyvinylpyrrolidone, hydroxypropyl methylcellulose, methylcellulose, and other protective excipients. In some examples, pH buffering agents such as phosphate buffers, citrate buffers, acetate buffers, and histidine buffers may be used to maintain optimal pH conditions. In some examples, ionic strength modifiers such as sodium chloride, potassium chloride, and other salts may be included to maintain protein stability and prevent aggregation.

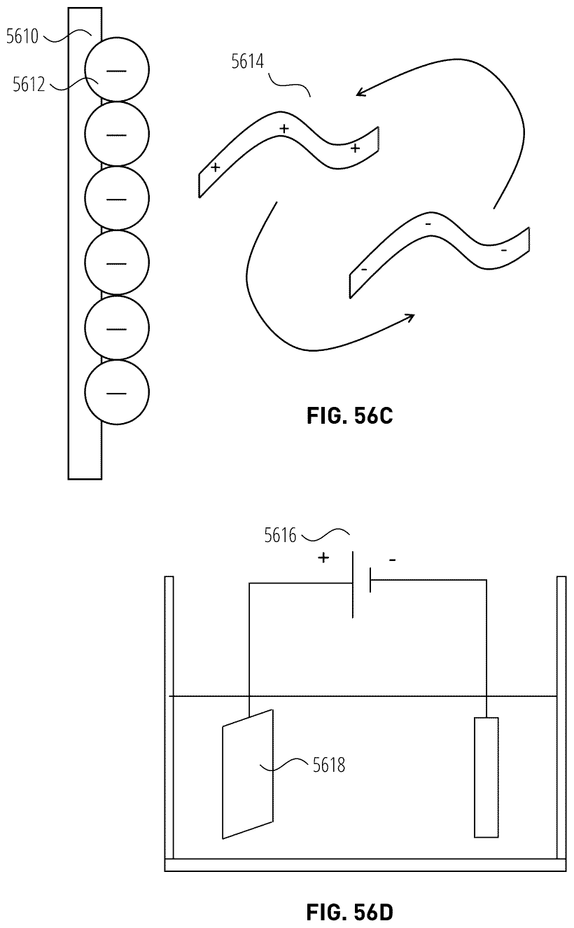

The therapeutic coating may be deposited or formed on the surface of the balloon, stent, or both, by any suitable application technique. In certain examples, spray coating methods are employed (as shown in FIG. 56A), whereby a solution or suspension of the drug and polymer is atomized and directed onto the surface (5604) of the device by a nozzle (5602). The spray process may utilize electrostatic charging of the droplets to promote uniform distribution and to enhance adhesion. In some examples, atmospheric plasma treatment may be employed to modify the surface energy and topography of the device prior to coating application, thereby enhancing adhesion and enabling controlled wetting characteristics, where the plasma treatment may utilize various gas compositions including argon, oxygen, nitrogen, or mixtures thereof.

In some examples, the device (5606) may be dipped into a coating bath (5608) containing the drug-polymer mixture, followed by evaporation of the solvent to form a continuous film (as shown in FIG. 56B). In some examples, inkjet printing or microdispensing techniques can be utilized to deposit discrete micro-droplets of coating composition in pre-defined patterns, such as stripes, chevrons, or radial bands. In some examples, layer-by-layer (LbL) assembly techniques (as shown in FIG. 56C) may be utilized, whereby alternating layers (5612) of oppositely charged polyelectrolytes (5614) are deposited on a base (5610) to create precisely controlled multilayer films, where each layer may incorporate different therapeutic agents, allowing for sequential or simultaneous release of multiple drugs.

In some examples, electrostatic or electrophoretic deposition (as shown in FIG. 56D) may be used, e.g., charged particles of drug or polymer are driven onto the substrate surface (5618) by an applied electrical field (5616). In certain examples, supercritical carbon dioxide may be used as a solvent or plasticizing medium to impregnate the drug into a porous polymer layer or to precipitate the drug into micro- or nano-structured morphologies. In some examples, focused ion beam (FIB) milling, or laser ablation techniques may be used to create micro-channels, wells, or surface texturing that serves as reservoirs for therapeutic agent loading, providing precise spatial control over drug placement and release kinetics. In some examples, cryogenic processing techniques may be utilized during coating application or post-treatment, where controlled cooling enhances drug stability, modifies polymer crystallinity, or creates unique morphological features that influence release characteristics.

In some examples, microcontact printing, in which a patterned stamp transfers the coating onto the surface, or plasma-enhanced chemical vapor deposition, may be used, by which a polymer or drug layer is deposited through ionized gas phase precursors. In some examples, the drug may be incorporated directly into the polymer matrix during extrusion or co-extrusion of the balloon or stent material, so that the therapeutic agent is dispersed throughout the device wall itself. In some examples, combination coating methods may be employed sequentially or simultaneously, such as combining spray coating with subsequent vapor deposition, or applying electrostatic deposition followed by solvent annealing to optimize coating uniformity and adhesion. In some examples, surface activation techniques such as corona discharge, flame treatment, or chemical etching may be applied to enhance coating adhesion, with the activation parameters optimized for specific device materials and coating compositions. In some examples, post-coating treatment steps may be employed, such as controlled annealing, UV curing, electron beam treatment, or chemical cross-linking, to optimize coating properties including adhesion strength, drug release kinetics, and mechanical durability.

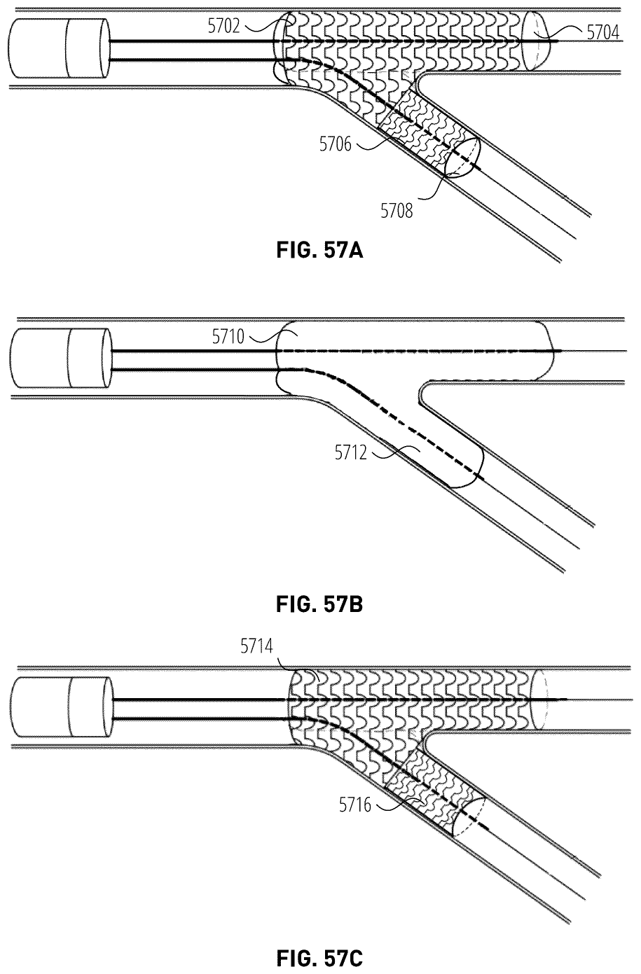

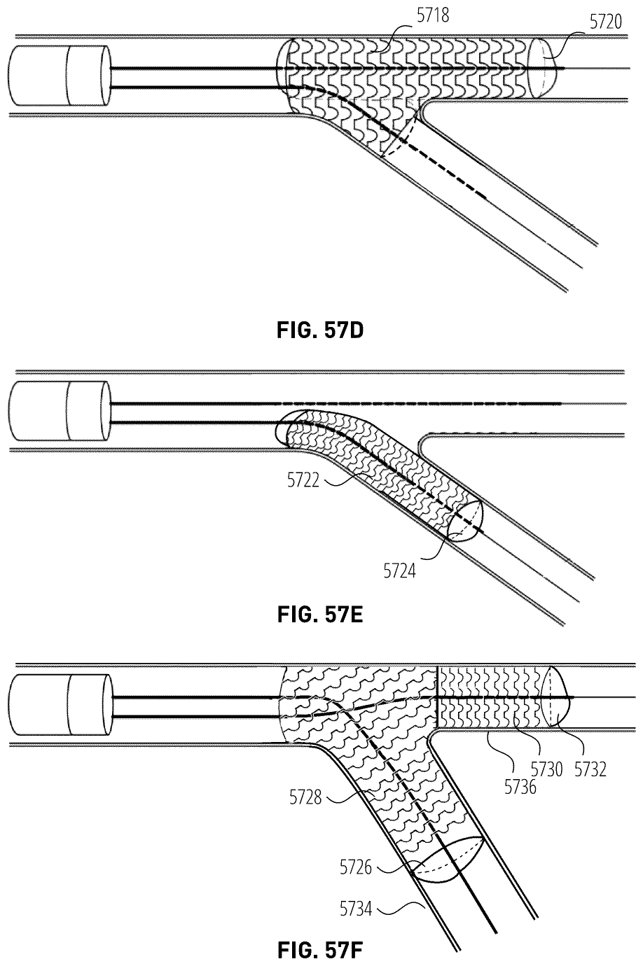

As shown in FIG. 57A, in some examples, the therapeutic coating may be applied to the entire surface of the balloon (e.g., mother balloon 5704, daughter balloon 5708) or stent (e.g., mother stent 5702, daughter ballon 5706), or may be selectively disposed only on predetermined regions specifically engineered to optimize therapeutic efficacy and minimize systemic exposure. For example, the coating fully covers the working length of the balloon and both the proximal and distal taper regions, thereby ensuring drug delivery along the entire dilated vessel segment and providing comprehensive therapeutic coverage during balloon expansion. In some examples, the coating is limited only to the central working length, leaving the tapers uncoated, which may be advantageous for avoiding drug contact with adjacent non-diseased tissue and reducing the potential for off-target effects. In some examples, the coating may be applied in a gradient pattern where the drug concentration varies along the length of the device, such that higher concentrations are positioned at regions of expected higher therapeutic need, while lower concentrations or drug-free zones are positioned at regions where therapeutic intervention is less critical. In some examples, the coating distribution may be customized based on anatomical landmarks, vessel geometry, or disease severity, allowing for personalized therapeutic delivery profiles that account for patient-specific factors and lesion characteristics.

As shown in FIG. 57B, mother balloon (5710) and/or daughter balloon (5712) may directly contact the walls of vessels and deliver the therapeutic agents without having the stents. As shown in FIG. 57C, mother stent (5714) and/or daughter stent (5716) may directly contact the walls of vessels and deliver the therapeutic agents without having the balloons. As shown in FIG. 57D, mother stent (5718) and/or mother balloon (5720) may contact the walls of mother vessel and deliver the therapeutic agents. As shown in FIG. 57E, daughter stent (5722) and/or daughter balloon (5724) may contact the walls of daughter vessel and deliver the therapeutic agents. As shown in FIG. 57F, in some examples when the bifurcation angle is greater than 60-70 degrees, the mother balloon (5726) and/or the mother stent (5728) may contact the daughter vessel (5734) and the daughter balloon (5732) and/or the daughter stent (5730) may contact the mother vessel (5736).

In some examples, the coating may be applied exclusively to the proximal taper, the distal taper, or discrete circumferential bands arranged along the balloon in patterns specifically designed to target particular anatomical features or therapeutic requirements. For instance, the balloon may include one or more radial or spiral segments enriched with higher drug content, configured to deliver enhanced therapeutic effect at specific anatomical landmarks such as a bifurcation or ostium, where restenosis rates are typically higher and more aggressive therapeutic intervention may be warranted. In some examples, the coating may be applied in alternating segments along the balloon length, creating drug-eluting and drug-free zones that allow for controlled release kinetics and reduced systemic exposure. In some examples, the coating may be configured in helical patterns that ensure uniform drug distribution during balloon rotation or advancement, or in longitudinal stripes that align with specific vessel wall regions based on pre-procedural imaging. In some examples, the coating pattern may include micro-deposits or nano-scale reservoirs arranged in geometric patterns such as hexagonal arrays, concentric circles, or fractal designs that optimize surface area contact and drug elution characteristics while maintaining balloon flexibility and deliverability.

Similarly, a stent may be fully coated over all struts and connector elements to provide comprehensive therapeutic coverage, or selectively coated only on portions of the struts while leaving the inter-strut spaces uncoated to allow for tissue ingrowth and endothelialization. In some examples, the stent coating may be concentrated around fenestrations or side holes to deliver the drug preferentially to a side branch or ostial lesion, where targeted therapeutic intervention is most needed. In some examples, the coating may be applied only to the luminal surface of the stent struts to maximize drug contact with blood flow and circulating cells, while the abluminal surface remains uncoated to facilitate tissue integration. In some examples, the coating distribution may vary circumferentially around the stent, with higher drug concentrations applied to struts positioned at predicted high-stress regions or areas of expected neointimal proliferation. In some examples, the stent coating may be applied in a temporal sequence during manufacturing, where different struts receive different drug formulations or concentrations to create a multi-drug delivery system that provides sequential or synergistic therapeutic effects over time.

In some examples, the phenomenon of “pillowing,” whereby the balloon protrudes beyond or between stent struts during inflation, provides a unique opportunity to deliver drug selectively to the margins of the stent and regions that would otherwise remain unscaffolded. Accordingly, in certain examples, the coating is applied to the anticipated pillow regions so that drug is released to unscaffolded vessel wall segments adjacent to the stent edges, ensuring therapeutic coverage of the entire treated vessel segment. In some examples, the coating in pillow regions may have different drug concentrations or release kinetics compared to other balloon regions, allowing for customized therapeutic delivery based on the expected contact time and mechanical forces in each region. In some examples, the pillow regions may be pre-formed or shaped during balloon manufacturing to optimize drug contact and delivery efficiency, or may include specialized surface textures or micro-structures that enhance drug transfer to the vessel wall. In some examples, the pillow region coating may include multiple therapeutic agents with different molecular weights or release characteristics, allowing for both immediate and sustained therapeutic effects at the stent margins where edge restenosis is a particular concern.

In examples incorporating dual expandable members, such as mother and daughter balloon configurations, the therapeutic coating may be applied in numerous combinations to optimize drug delivery across the bifurcated vessel system. For example, both the mother balloon and daughter balloon may carry identical therapeutic agents at equivalent concentrations, providing uniform drug delivery throughout the entire treated vessel segment. In some examples, the mother balloon may carry a first therapeutic agent while the daughter balloon carries a second, different therapeutic agent, allowing for synergistic therapeutic effects or targeting of different pathological processes within the same bifurcation. In some examples, the therapeutic agent concentration may vary between the mother and daughter balloons, such that the daughter balloon carries a higher concentration for treating more severe side branch stenosis, while the mother balloon carries a lower concentration for maintenance therapy in the main vessel. In some examples, the drug release kinetics may be differentiated between the two balloons, where the daughter balloon provides rapid drug release for immediate therapeutic effect, while the mother balloon provides sustained release for long-term restenosis prevention. In some examples, the coating may be applied to only one balloon while the other remains drug-free, allowing for selective therapeutic intervention in either the main vessel or side branch as clinically indicated.

Similarly, in dual-stent configurations, the therapeutic coating may be strategically distributed between the mother stent and daughter stent to optimize therapeutic coverage and minimize systemic exposure. In some examples, both stents may carry identical therapeutic agents with matching release profiles, ensuring consistent drug delivery across the entire scaffolded region. In some examples, the mother stent may be coated with an anti-proliferative agent such as paclitaxel, while the daughter stent carries an anti-inflammatory agent such as dexamethasone, providing complementary therapeutic mechanisms. In some examples, the drug loading may be concentrated on the stent struts that contact the vessel wall, while connector elements remain uncoated to facilitate endothelialization and reduce thrombogenicity. In some examples, the mother stent may carry a higher drug load in regions adjacent to the side hole, where stent overlap occurs and restenosis risk is elevated, while maintaining standard drug loading in other regions. In some examples, the coating may be applied in a gradient pattern along the stent length, with higher concentrations at the proximal and distal ends where mechanical stress is greatest, and lower concentrations in the central region where drug diffusion from adjacent components may supplement therapeutic levels.

In hybrid configurations combining coated balloons with coated stents, the therapeutic delivery may be orchestrated to provide both immediate and sustained drug release with complementary temporal profiles. In some examples, the mother balloon may carry a rapidly-eluting therapeutic agent for immediate vessel wall treatment, while the mother stent provides sustained drug release over weeks to months for long-term restenosis prevention. In some examples, the daughter balloon may deliver a bolus dose of therapeutic agent during initial expansion, followed by continuous low-level release from the daughter stent throughout the healing process. In some examples, the balloon coating may include penetration enhancers or permeabilization agents that facilitate uptake of therapeutic agents subsequently released from the stent coating. In some examples, the stent coating may be designed to release different therapeutic agents at different time points, coordinated with the balloon's immediate drug delivery to provide sequential therapeutic intervention. In some examples, the balloon and stent may carry complementary drug combinations, such as an anti-proliferative agent on the balloon for immediate smooth muscle cell inhibition, paired with an anti-inflammatory agent on the stent for sustained inflammatory response modulation.

The spatial relationship between coated components may be optimized to ensure complete therapeutic coverage while minimizing drug-drug interactions and systemic exposure. In some examples, the mother balloon coating may be positioned to align with uncoated regions of the daughter stent, ensuring no gap in therapeutic coverage while avoiding excessive drug concentration in overlap zones. In other examples, the daughter balloon coating may be concentrated in the distal region that extends beyond the mother stent, providing dedicated therapeutic treatment to the side branch vessel. In some examples, the coating distribution may be designed to account for balloon “pillowing” effects, where drug-eluting regions of the balloon contact vessel wall areas not covered by stent struts, ensuring comprehensive therapeutic coverage of the entire treated vessel segment. In some examples, the mother and daughter components may be coated with different therapeutic agents that exhibit synergistic effects when delivered to adjacent tissue regions, such as combining anti-proliferative and pro-healing agents to optimize the balance between restenosis prevention and vessel healing.

Advanced coating techniques may be employed to create component-specific release profiles that account for the unique mechanical and hemodynamic environment of each device. In some examples, the mother balloon coating may be formulated to withstand higher inflation pressures and longer deployment times typical of main vessel treatment, while the daughter balloon coating may be optimized for the lower pressures and shorter contact times typical of side branch interventions. In other examples, the coating adhesion and mechanical properties may be tailored to each component's specific expansion characteristics, vessel contact patterns, and withdrawal requirements. In some examples, the therapeutic agents may be incorporated into responsive polymer systems that react to local environmental conditions, such as pH changes, enzyme activity, or mechanical stress, allowing for adaptive drug release based on the healing response of each vessel segment. In some examples, the coating may include targeting moieties or adhesion promoters that enhance drug retention and uptake specific to the cellular environment of either main vessel or side branch anatomy.

Quality control and manufacturing considerations for dual-component systems may include specialized coating processes that ensure consistent drug loading, uniform distribution, and appropriate release characteristics across all therapeutic surfaces. In some examples, the coating process may involve sequential application steps, where each component is coated separately to optimize drug loading and coating quality for its specific geometry and functional requirements. In some examples, simultaneous coating processes may be employed using multi-nozzle spray systems or selective masking techniques to achieve precise drug distribution patterns across multiple components. In some examples, the coating validation may include component-specific testing protocols that verify drug content, release kinetics, and coating integrity for each balloon and stent independently, as well as system-level testing to confirm appropriate therapeutic delivery when components are used in combination. In some examples, the manufacturing process may incorporate real-time monitoring of coating parameters for each component, allowing for dynamic adjustment of coating conditions to maintain consistent quality across all therapeutic surfaces in the dual-component system.

The balloon component may be configured in numerous ways to optimize mechanical deployment and drug delivery, with designs ranging from simple single-lumen constructions to complex multi-chamber systems that provide precise control over expansion characteristics. In some examples, the balloon comprises a single-lumen design that inflates uniformly across its length, providing consistent drug delivery pressure and contact time throughout the treated vessel segment. In some examples, the balloon includes multiple lumens or chambers that can be inflated independently, enabling differential expansion along the length or circumference of the balloon to accommodate vessel tapering, side branch anatomy, or varying lesion severity. For example, a dual-lumen balloon may have a central working segment that inflates to a larger diameter for main vessel treatment, with proximal and distal tapers remaining at a smaller diameter or inflating in sequence to provide controlled drug delivery timing and minimize vessel trauma. In some examples, the balloon may include pressure-sensing capabilities that allow for real-time monitoring of inflation pressure and automatic adjustment of drug delivery parameters based on vessel compliance and resistance. In some examples, the balloon may be designed with flow-through capabilities that allow for simultaneous drug infusion and balloon inflation, providing enhanced drug delivery efficiency and reduced procedure time.

In some examples, the balloon is constructed of materials exhibiting different compliance characteristics along its length, such that certain regions preferentially expand under pressure to accommodate varying vessel diameters and optimize drug delivery contact. These materials may include combinations of high-compliance polymers in regions requiring significant expansion and low-compliance materials in regions where precise size control is critical. Other configurations may include segmented balloons divided into discrete inflatable compartments, each capable of independent pressure control and drug delivery. In some examples, the balloon material may incorporate shape-memory alloys or polymers that provide predictable expansion characteristics at body temperature, ensuring consistent performance across different patient anatomies and environmental conditions. In some examples, the balloon may be constructed with reinforcing elements such as embedded fibers or mesh structures that provide directional expansion control and prevent over-inflation while maintaining flexibility for navigation through tortuous anatomy. In some examples, the balloon material may include biodegradable components that dissolve over time, allowing for temporary mechanical support during acute treatment followed by complete absorption to minimize long-term foreign body presence.

Certain examples incorporate porous or microporous balloon walls, enabling liquid formulations of the therapeutic agent to infuse directly into the vessel wall during inflation, providing enhanced drug penetration and tissue distribution compared to surface-applied coatings. The pore size and distribution may be precisely controlled during manufacturing to optimize drug diffusion rates and prevent particle migration while maintaining balloon integrity under inflation pressures. In some configurations, the balloon surface may be textured, for example with micro-grooves, ridges, or nodules to enhance friction, improve contact area, or augment coating adhesion and drug transfer efficiency. In some examples, the surface texturing may be created through laser etching, chemical etching, or molding processes that create microscale features ranging from 0.1 to 100 micrometers in depth and spacing. In some examples, the textured surface may include hierarchical structures with both microscale and nanoscale features that provide multiple mechanisms for drug retention and release. In some examples, the balloon surface may be coated with biocompatible adhesives or coupling agents that enhance drug binding and control release kinetics, or may include surface-grafted polymer chains that provide sustained drug elution over extended periods.

In some examples, micro-needle arrays can be integrated into the balloon surface, configured to penetrate the intima and deliver drug intramurally upon balloon inflation, providing direct tissue injection and enhanced bioavailability compared to luminal drug delivery. The micro-needles may be fabricated from biodegradable materials such as polylactic acid or polycaprolactone, allowing for temporary tissue penetration followed by complete absorption. In some examples, the micro-needle arrays may be loaded with different therapeutic agents or concentrations, enabling multi-drug delivery or dose-dependent therapeutic effects based on penetration depth and tissue type. In some examples, the micro-needles may be designed with hollow cores or internal channels that allow for pressurized drug injection during balloon inflation, providing active drug delivery rather than passive diffusion. In some examples, the micro-needle arrays may be arranged in specific patterns or densities that correspond to vessel anatomy, lesion characteristics, or therapeutic requirements, allowing for customized treatment approaches. In some examples, the micro-needles may include dissolution-controlling coatings or drug-release mechanisms that provide sustained therapeutic delivery over days or weeks following implantation.

The stent may be selected or adapted from numerous designs and materials, each offering specific advantages for different clinical applications and patient anatomies. For example, in some examples, the stent comprises an open-cell architecture, facilitating flexibility and conformability to tortuous vessels while allowing for side branch access and optimal drug delivery to complex lesion geometries. In some examples, the stent includes a closed-cell design to provide higher radial strength and scaffolding support for calcified or fibrotic lesions that require more aggressive mechanical intervention. In some examples, the stent may incorporate hybrid designs that combine open-cell regions for flexibility with closed-cell regions for strength, optimizing performance for specific anatomical requirements. In some examples, the stent material may include nitinol, stainless steel, cobalt-chromium alloys, or platinum-iridium composites, each selected for specific mechanical properties, biocompatibility, and radiopacity requirements. In some examples, the stent may be surface-treated or coated with bioactive agents such as anti-thrombotic compounds, endothelialization-promoting factors, or anti-inflammatory agents that complement the primary therapeutic coating and enhance overall clinical outcomes.

In some examples, stents may incorporate fenestrations or side windows, specifically configured to align with a side branch ostium, enabling direct delivery of drug or passage of secondary devices while maintaining optimal therapeutic coverage of the main vessel. These fenestrations may be pre-formed during manufacturing or created in situ during deployment using specialized tools or techniques. Other examples may employ stents having variable strut thickness or radial strength along their length, such that different portions expand to different diameters to match vessel tapering or accommodate varying lesion severity. In some examples, the stent may include articulating segments or hinged connections that allow for conformability to vessel curvature while maintaining structural integrity and drug delivery capabilities. In some examples, the stent design may incorporate flow-directing elements or baffles that optimize drug distribution and minimize systemic exposure by directing therapeutic agents toward the vessel wall rather than into the bloodstream. In some examples, the stent may include integrated sensors or monitoring devices that provide real-time feedback on drug delivery, tissue response, or healing progress, allowing for personalized treatment adjustments and improved clinical outcomes.

In some examples, the stent comprises bioresorbable materials such as polylactic acid, polyglycolic acid, or magnesium alloys, designed to gradually degrade after fulfilling their mechanical and therapeutic function, eliminating the need for permanent implants and reducing long-term complications. The degradation rate may be controlled through material composition, processing conditions, or surface treatments to match the expected healing timeline and provide optimal therapeutic coverage throughout the critical healing period. Radiopaque markers may be included on the stent ends or adjacent to side holes to facilitate precise visualization and alignment during deployment, ensuring accurate positioning and optimal therapeutic delivery. In some examples, the bioresorbable stent may be designed with variable degradation rates along its length, allowing for prolonged support in high-stress regions while enabling faster absorption in areas where early tissue remodeling is desired. In some examples, the stent may include protective coatings or barrier layers that control the degradation process and prevent premature drug release or structural failure. In some examples, the radiopaque markers may be integrated into the stent structure rather than attached as separate components, providing permanent visualization capabilities even after stent degradation. In some examples, the stent may be designed with retrievable elements or features that allow for partial or complete removal if adverse events occur or if alternative treatments become necessary.

In any embodiment, the device may carry a single therapeutic agent or a combination of two or more agents configured for complementary pharmacologic effects. For example, the coating may include an antiproliferative agent such as sirolimus (also known as Rapamycin), everolimus, zotarolimus, temsirolimus, ridaforolimus, pimecrolimus, tacrolimus, biolimus, deforolimus, paclitaxel, docetaxel, cabazitaxel, nab-paclitaxel, abraxane, myolimus, novolimus, umirolimus, biolimus A9, amphimus, and other analogs and derivatives of rapamycin and taxanes. In some examples, the coating may include anti-inflammatory agents such as dexamethasone, methylprednisolone, prednisolone, betamethasone, triamcinolone, hydrocortisone, budesonide, fluticasone, prednisone, cortisone, deflazacort, and other glucocorticoids and corticosteroids. In some examples, the coating may include antithrombotic agents such as heparin, low molecular weight heparin, enoxaparin, fondaparinux, rivaroxaban, apixaban, dabigatran, warfarin, urokinase, tissue plasminogen activator (tPA), streptokinase, and other anticoagulants, thrombolytics, and fibrinolytics. In some examples, the coating may include antiplatelet agents such as aspirin, clopidogrel, ticagrelor, prasugrel, abciximab, eptifibatide, tirofiban, dipyridamole, and other platelet aggregation inhibitors. In some examples, the coating may include antihyperlipidemic agents such as atorvastatin, simvastatin, rosuvastatin, pravastatin, lovastatin, fluvastatin, pitavastatin, probucol, ezetimibe, and other statins and cholesterol-lowering agents. In some examples, the coating may include cytotoxic antibiotics such as actinomycin-D, mitomycin C, doxorubicin, daunorubicin, bleomycin, and other antineoplastic antibiotics. In some examples, the coating may include antimetabolites such as methotrexate, 5-fluorouracil, gemcitabine, cytarabine, and other DNA synthesis inhibitors. In some examples, the coating may include antimicrobial agents such as gentamicin, vancomycin, ciprofloxacin, minocycline, rifampin, silver sulfadiazine, chlorhexidine, and other antibiotics, antifungals, and antiseptics. In some examples, the coating may include antioxidants such as vitamin E, ascorbic acid, probucol, butylated hydroxytoluene (BHT), and other free radical scavengers. In some examples, the coating may include vasodilators such as nitric oxide donors, nitroprusside, nitroglycerin, isosorbide dinitrate, papaverine, and other smooth muscle relaxants. In some examples, the coating may include hormones such as estrogen, estradiol, progesterone, testosterone, and other steroid hormones. In some examples, the coating may include antiarrhythmic agents such as amiodarone, lidocaine, procainamide, quinidine, and other cardiac rhythm modulators. In some examples, the coating may include calcium channel blockers such as nifedipine, verapamil, diltiazem, amlodipine, and other calcium antagonists. In some examples, the coating may include ACE inhibitors such as captopril, enalapril, lisinopril, ramipril, and other angiotensin-converting enzyme inhibitors. In some examples, the coating may include angiotensin receptor blockers such as losartan, valsartan, irbesartan, candesartan, and other ARBs. In some examples, the coating may include beta-blockers such as metoprolol, propranolol, atenolol, carvedilol, and other beta-adrenergic antagonists. In some examples, the coating may include anti-allergic agents such as cromolyn sodium, nedocromil, and other mast cell stabilizers. In some examples, the coating may include local anesthetics such as lidocaine, bupivacaine, procaine, and other sodium channel blockers. In some examples, the coating may include anti-adhesion agents such as hyaluronic acid, phosphorylcholine, and other biocompatible polymers that prevent protein and cellular adhesion.

The multiple agents may be co-dissolved in a common matrix, arranged in separate discrete layers, or spatially separated along the length or circumference of the device to provide customized therapeutic delivery profiles tailored to specific anatomical requirements and disease states. For instance, one drug may be localized to the proximal taper while another is concentrated at the distal end, allowing for targeted therapeutic intervention at different regions of the treated vessel segment. In some examples, the multiple agents may be arranged in alternating bands or stripes along the device length, creating zones of different therapeutic activity that correspond to specific vessel anatomy or lesion characteristics. In some examples, the agents may be distributed in a gradient pattern where the concentration of each agent varies continuously along the device, such that one agent predominates in the proximal region while another predominates distally, with a transitional zone where both agents are present in varying ratios. In some examples, the multiple agents may be encapsulated in separate microparticles or nanoparticles that are then distributed within the coating matrix, allowing for independent release kinetics and therapeutic effects. In some examples, the agents may be incorporated into different polymer layers of a multilayer coating system, where each layer provides distinct release characteristics and therapeutic timing. In some examples, the spatial separation may be achieved through selective masking during the coating process, allowing for precise control over drug placement and concentration gradients. In some examples, the multiple agents may include synergistic combinations such as anti-proliferative and anti-inflammatory compounds, or complementary agents such as vasodilators paired with anti-thrombotic compounds to address multiple aspects of vascular healing and restenosis prevention.

The drug release profile can be tailored to achieve a rapid burst release, sustained release over a prolonged period, or a biphasic pattern combining both, depending on the specific clinical indication and desired therapeutic outcome. For example, in one embodiment, at least 70% of the drug may be eluted within the first hour after deployment to provide immediate therapeutic intervention, while in another embodiment, the coating is configured for gradual elution over 7 to 90 days to maintain therapeutic levels throughout the critical healing period. In some examples, the release profile may be triphasic, with an initial rapid release phase for immediate therapeutic effect, followed by a sustained release phase for maintenance therapy, and concluding with a final extended release phase for long-term restenosis prevention. In some examples, the burst release may be controlled to occur within specific time windows, such as within the first 15 minutes, 30 minutes, or 60 minutes after deployment, allowing for precise timing of therapeutic intervention. In some examples, the sustained release portion may be modulated to provide different release rates at different time points, such as higher release rates during the first week followed by lower maintenance rates for subsequent weeks. In some examples, the biphasic pattern may be achieved through the use of different polymer coatings or drug formulations that respond to different environmental triggers or degradation mechanisms. In some examples, the release profile may be designed to match the natural healing timeline of the vessel, with higher drug concentrations released during periods of active inflammatory response and lower concentrations during tissue remodeling phases. In some examples, the coating may be configured to provide pulsatile release, where drug is released in controlled bursts at predetermined intervals to maintain therapeutic efficacy while minimizing systemic exposure.

The elution characteristics may be further controlled by selecting the coating porosity, thickness, polymer crystallinity, hydrophilicity or hydrophobicity, and the presence of pore-formers, plasticizers, or crosslinking agents to fine-tune the mechanical properties, drug loading capacity, and release kinetics. In some examples, the release profile may be triggered or modulated by environmental conditions such as pH, enzymatic activity, or temperature, allowing for adaptive therapeutic delivery that responds to local tissue conditions. In some examples, the coating porosity may be graded along the device length, with higher porosity in regions requiring rapid drug release and lower porosity in regions requiring sustained release. In some examples, the polymer crystallinity may be controlled through thermal treatment or solvent selection during coating application, allowing for precise control over drug diffusion pathways and release rates. In some examples, the hydrophilicity or hydrophobicity of the coating may be adjusted through the incorporation of specific functional groups or surface treatments that alter the interaction between the coating and biological fluids. In some examples, pore-formers such as polyvinyl alcohol, polyethylene glycol, or sodium chloride may be incorporated into the coating and subsequently removed through dissolution or leaching, creating controlled porosity networks that facilitate drug release. In some examples, crosslinking agents such as glutaraldehyde, formaldehyde, or photo-crosslinkable compounds may be used to create covalent bonds within the polymer network, providing enhanced coating stability and controlled drug release. In some examples, the environmental triggers may include specific pH ranges associated with inflammatory conditions, enzymatic activity from activated macrophages or smooth muscle cells, or temperature variations associated with increased blood flow or metabolic activity.

As mentioned above, when the balloon is inflated within a stent, portions of the balloon may protrude between or beyond the stent struts to form so-called pillow regions that provide unique opportunities for targeted drug delivery to areas that would otherwise remain unscaffolded. These pillow regions may be selectively coated with a higher concentration of the therapeutic agent to deliver drug directly to vessel wall areas that are not mechanically supported by the stent, ensuring comprehensive therapeutic coverage of the entire treated vessel segment. This localized delivery may be particularly beneficial to reduce restenosis or hyperplasia at the stent margins or adjacent to ostial side branches, where mechanical support is limited and the risk of tissue proliferation is elevated. In some examples, the drug concentration in pillow regions may be 2-10 times higher than on other balloon surfaces to compensate for the shorter contact time and ensure adequate drug transfer to the vessel wall. In some examples, the pillow regions may be coated with specialized adhesion promoters or penetration enhancers that improve drug retention and uptake in these critical areas. In some examples, the coating in pillow regions may be designed to dissolve more rapidly than coatings on other balloon surfaces, ensuring immediate drug release upon contact with the vessel wall. In some examples, the pillow regions may be strategically positioned to align with specific anatomical features such as bifurcation points, vessel curves, or areas of known high mechanical stress where enhanced therapeutic intervention is most needed.

Any of the features described herein, including but not limited to coating structures, drug compositions, spatial arrangements, balloon configurations, stent designs, and release profiles, may be combined in any permutation or combination to create customized therapeutic delivery systems that address the specific requirements of individual patients and clinical scenarios. For example, a dual-lumen balloon having micro-needles and a hydrogel-based gradient coating may be used in conjunction with a bioresorbable stent partially coated on selected struts to provide both immediate mechanical support and sustained therapeutic delivery. In some examples, a porous balloon with a microparticle matrix configured for burst release may be used without any stent at all, relying solely on pharmacological intervention to achieve therapeutic outcomes. In some examples, a balloon coated only on the distal taper and anticipated pillow regions may be paired with a stent coated solely on the central segment adjacent to a bifurcation, creating a complementary therapeutic delivery system that addresses both main vessel and side branch requirements. In some examples, the combination may include multiple balloons with different coating patterns, such as a mother balloon with sustained-release coating paired with a daughter balloon carrying burst-release formulation. In some examples, the system may incorporate both drug-eluting and drug-free components, where therapeutic agents are selectively placed only in regions of highest clinical need while maintaining optimal mechanical performance in other areas. In some examples, the combinations may include different stent materials paired with different balloon coatings, such as metallic stents with anti-proliferative balloon coatings or bioresorbable stents with anti-inflammatory balloon coatings. In some examples, the permutations may include temporal variations, where different components are activated or deployed at different times during the procedure to provide sequential therapeutic intervention. These examples are illustrative only and are not limiting, as the modular nature of the system allows for virtually unlimited combinations tailored to specific clinical requirements and patient anatomies.

### NOTES AND EXAMPLES

The following, non-limiting examples, detail certain aspects of the present subject matter to solve the challenges and provide the benefits discussed herein, among others.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

Example 1 is a method for treating a vessel with a bifurcation, the vessel having a mother vessel and a daughter vessel, the method comprising: providing a delivery system having a mother catheter with a mother expandable member coupled to a distal portion of a mother elongate shaft, and a daughter catheter with a daughter expandable member coupled to a distal portion of a daughter elongate shaft; advancing the delivery system toward the bifurcation so that the mother expandable member is at least partially disposed in the mother vessel, and the daughter expandable member is at least partially disposed in the daughter vessel; radially expanding the daughter expandable member into engagement with a wall of the daughter vessel and eluting a therapeutic agent from the daughter expandable member into the wall, wherein a portion of the daughter expandable member carries the therapeutic agent and a portion of the daughter expandable member remains free of the therapeutic agent.

Example 2 is the method of Example 1, wherein the daughter expandable member remains free of a stent.

Example 3 is the method of any of Examples 1-2, wherein the therapeutic agent is one or more of an anti-platelet agent, anti-inflammatory agent, anti-hyperlipidemic agent, anti-proliferative agent, an antibiotic, or anti-thrombogenic agent.

Example 4 is the method of any of Examples 1-3, wherein the therapeutic agent comprises paclitaxel.

Example 5 is the method of any of Examples 1-4, wherein the therapeutic agent is coupled with an excipient.

Example 6 is the method of any of Examples 1-5, wherein the therapeutic agent is only carried on a distal portion of the daughter expandable member, and wherein a proximal portion of the daughter expandable member remains free of the therapeutic agent.

Example 7 is the method of any of Examples 1-6, wherein a tapered region of the daughter expandable member carries the therapeutic agent.

Example 8 is the method of any of Examples 1-7, wherein a tapered region of the daughter expandable member remains free of the therapeutic agent.

Example 9 is the method of any of Examples 1-8, wherein the daughter expandable member is a balloon having a distal tapered region, a proximal tapered region, and a working length disposed therebetween, and wherein the therapeutic agent is disposed only on a distal portion of the working length, and the proximal tapered region and the distal tapered region remain free of the therapeutic agent.

Example 10 is the method of any of Examples 1-10, wherein the mother expandable member or the daughter expandable member is a balloon.

Example 11 is the method of any of Examples 1-10, wherein the daughter catheter further comprises a proximal marker, a distal marker, and an intermediate marker, each disposed on the daughter elongate shaft, the proximal, distal, and intermediate markers configured to be observed under an imaging modality, wherein the proximal marker is axially aligned with a proximal portion of a working length of the daughter expandable member, wherein the distal marker is axially aligned with a distal portion of a working length of the daughter expandable member, the distal portion of the working length carrying the therapeutic agent, and wherein the intermediate marker is disposed between the proximal marker and the distal marker, and the intermediate marker is axially aligned with a proximal edge of the distal portion of the working length where the daughter expandable member stops carrying the therapeutic agent.

Example 12 is a system for treating a vessel with a bifurcation, the vessel having a mother vessel and a daughter vessel, the system comprising: a mother catheter comprising a mother expandable member and a mother elongate shaft, the mother expandable member coupled to a distal portion of the mother elongate shaft; a daughter catheter comprising a daughter expandable member and a daughter elongate shaft, the daughter expandable member coupled to a distal portion of the daughter elongate shaft; and a therapeutic agent carried on the daughter expandable member, wherein radial expansion of the daughter expandable member is configured to engage the daughter expandable member with a wall of the daughter vessel and the therapeutic agent elutes from the daughter expandable member into the wall.

Example 13 is the system of Example 12, wherein the therapeutic agent is one or more of an anti-platelet agent, anti-inflammatory agent, anti-hyperlipidemic agent, anti-proliferative agent, an antibiotic, or anti-thrombogenic agent.

Example 14 is the system of any of Examples 12-13, wherein the therapeutic agent comprises paclitaxel.

Example 15 is the system of any of Examples 12-14, wherein the therapeutic agent is coupled with an excipient.

Example 16 is the system of any of Examples 12-15, wherein the therapeutic agent is only carried on a distal portion of the daughter expandable member, and wherein a proximal portion of the daughter expandable member remains free of the therapeutic agent.

Example 17 is the system of any of Examples 12-16, wherein a tapered region of the daughter expandable member carries the therapeutic agent.

Example 18 is the system of any of Examples 12-17, wherein a tapered region of the daughter expandable member remains free of the therapeutic agent.

Example 19 is the system of any of Examples 12-18, wherein the daughter expandable member is a balloon having a distal tapered region, a proximal tapered region, and a working length disposed therebetween, and wherein the therapeutic agent is disposed only on a distal portion of the working length, and the proximal tapered region and the distal tapered region remain free of the therapeutic agent.

Example 20 is the system of any of Examples 12-19, wherein the mother expandable member or the daughter expandable member is a balloon.

Example 21 is the system of any of Examples 12-20, wherein the daughter catheter further comprises a proximal marker, a distal marker, and an intermediate marker, each disposed on the daughter elongate shaft, wherein the proximal marker is axially aligned with a proximal portion of a working length of the daughter expandable member, wherein the distal marker is axially aligned with a distal portion of a working length of the daughter expandable member, the distal portion of the working length carrying the therapeutic agent, and wherein the intermediate marker is disposed between the proximal marker and the distal marker, and the intermediate marker is axially aligned with a proximal edge of the distal portion of the working length where the daughter expandable member stops carrying the therapeutic agent.

Example 22 is a method for treating a vessel with a bifurcation, the vessel having a main vessel and a branch vessel, the method comprising: providing a delivery system having a first catheter with a first expandable member coupled to a distal portion of a first elongate shaft, and a second catheter with a second expandable member coupled to a distal portion of a second elongate shaft; advancing the delivery system toward the bifurcation so that the first expandable member is at least partially disposed in the main vessel, and the second expandable member is at least partially disposed in the branch vessel; radially expanding both the first expandable member and the second expandable member into engagement with walls of the main vessel and branch vessel; eluting one or more therapeutic agents from at least one of the first expandable member, or the second expandable member.

Example 23 is the method of Example 22, wherein the one or more therapeutic agents are eluted from both the first expandable member and the second expandable member.

Example 24 is the method of Example 22, wherein the one or more therapeutic agents is eluted only from the first expandable member, and the second expandable member remains free of therapeutic agents.

Example 25 is the method of Example 22, wherein the one or more therapeutic agent is eluted only from the second expandable member, and the first expandable member remains free of therapeutic agents.

Example 26 is the method of Example 23, wherein the one or more therapeutic agents eluted from the first expandable member and the second expandable member are identical.

Example 27 is the method of Example 23, wherein the one or more therapeutic agents eluted from the first expandable member and the second expandable member are different.

Example 28 is the method of Example 27, wherein the first expandable member elutes an anti-proliferative agent, and the second expandable member elutes an anti-inflammatory agent.

Example 29 is the method of Example 27, wherein the first expandable member elutes a first therapeutic agent configured for sustained release and the second expandable member elutes a second therapeutic agent configured for burst release.

Example 30 is the method of any of Examples 23 and 26-29, wherein a concentration of the therapeutic agent on the first expandable member is different from a concentration of the therapeutic agent on the second expandable member.

Example 31 is the method of any of Examples 22-30, wherein the one or more therapeutic agents comprise a combination of two or more agents selected from anti-platelet agents, anti-inflammatory agents, anti-hyperlipidemic agents, anti-proliferative agents, antibiotics, and anti-thrombogenic agents.

Example 32 is a system for treating a vessel with a bifurcation, the vessel having a main vessel and a branch vessel, the system comprising: a first catheter comprising a first expandable member and a first elongate shaft, the first expandable member coupled to a distal portion of the first elongate shaft; a second catheter comprising a second expandable member and a second elongate shaft, the second expandable member coupled to a distal portion of the second elongate shaft; and one or more therapeutic agents carried on at least one of the second expandable member or the first expandable member, wherein radial expansion of the second expandable member or the first expandable member is configured to engage the second expandable member or the first expandable member with a wall of the branch vessel or a wall of the main vessel and the one or more therapeutic agents elutes from the second expandable member or the first expandable member into the corresponding wall of the branch vessel or the main vessel.

Example 33 is the system of Example 32, wherein the one or more therapeutic agents are eluted from both the first expandable member and the second expandable member.

Example 34 is the system of Example 32, wherein the one or more therapeutic agents is eluted only from the first expandable member, and the second expandable member remains free of therapeutic agents.

Example 35 is the system of Example 32, wherein the one or more therapeutic agent is eluted only from the second expandable member, and the first expandable member remains free of therapeutic agents.

Example 36 is the system of Example 33, wherein the one or more therapeutic agents eluted from the first expandable member and the second expandable member are identical.

Example 37 is the system of Example 33, wherein the one or more therapeutic agents eluted from the first expandable member and the second expandable member are different.

Example 38 is the system of Example 37, wherein the first expandable member elutes an anti-proliferative agent, and the second expandable member elutes an anti-inflammatory agent.

Example 39 is the system of Example 37, wherein the first expandable member elutes a first therapeutic agent configured for sustained release and the second expandable member elutes a second therapeutic agent configured for burst release.

Example 40 is the system of any of Examples 33 and 36-39, wherein a concentration of the therapeutic agent on the first expandable member is different from a concentration of the therapeutic agent on the second expandable member.

Example 41 is the system of any of Example 32-40, wherein the one or more therapeutic agents comprise a combination of two or more agents selected from anti-platelet agents, anti-inflammatory agents, anti-hyperlipidemic agents, anti-proliferative agents, antibiotics, and anti-thrombogenic agents.

Example 42 is a method for treating a vessel with a bifurcation, the vessel having a main vessel and a branch vessel, the method comprising: providing a delivery system having a first catheter with a first stent disposed over a first expandable member, and a second catheter with a second stent disposed over a second expandable member; advancing the delivery system toward the bifurcation so that the first stent is positioned in the main vessel and the second stent is positioned in the branch vessel; radially expanding both the first stent and the second stent into engagement with walls of the main vessel and branch vessel; and eluting one or more therapeutic agents from at least one of the first stent, the first expandable member, the second stent, or the second expandable member.

Example 43 is the method of claim 42, wherein the one or more therapeutic agents are eluted from the first stent and the second stent.

Example 44 is the method of claim 42, wherein the one or more therapeutic agents are eluted from the first stent and the first expandable member.

Example 45 is the method of claim 42, wherein the one or more therapeutic agents are eluted from the first expandable member and the second expandable member.

Example 46 is the method of claim 42, wherein the one or more therapeutic agents are eluted from the second stent and the second expandable member.

Example 47 is a system for treating a vessel with a bifurcation, the vessel having a main vessel and a branch vessel, the system comprising: a first catheter comprising a first expandable member, a first stent, and a first elongate shaft, the first stent disposed over a first expandable member; a second catheter comprising a second expandable member, a second stent, and a second elongate shaft, the second stent disposed over a second expandable member; and one or more therapeutic agents carried on at least one of the second expandable member, the second stent, the first stent, or the first expandable member, wherein radial expansion of the second expandable member or the first expandable member is configured to engage the second stent or the first stent with a wall of the branch vessel or a wall of the main vessel and the one or more therapeutic agents elutes from the second expandable member or the first expandable member into the corresponding wall of the branch vessel or the main vessel.

Example 48 is the system of claim 47, wherein the one or more therapeutic agents are eluted from the first stent and the second stent.

Example 49 is the system of claim 47, wherein the one or more therapeutic agents are eluted from the first stent and the first expandable member.

Example 50 is the system of claim 47, wherein the one or more therapeutic agents are eluted from the first expandable member and the second expandable member.

Example 51 is the system of claim 47, wherein the one or more therapeutic agents are eluted from the second stent and the second expandable member.

In Example 52, the systems, apparatuses, or methods of any one or any combination of Examples 1-51 can optionally be configured such that all elements or options recited are available to use or select from.

1. A method for treating a vessel with a bifurcation, the vessel having a mother vessel and a daughter vessel, the method comprising:
providing a delivery system having a mother catheter with a mother expandable member coupled to a distal portion of a mother elongate shaft, and a daughter catheter with a daughter expandable member coupled to a distal portion of a daughter elongate shaft;
advancing the delivery system toward the bifurcation so that the mother expandable member is at least partially disposed in the mother vessel, and the daughter expandable member is at least partially disposed in the daughter vessel;
radially expanding the daughter expandable member into engagement with a wall of the daughter vessel and eluting a therapeutic agent from the daughter expandable member into the wall,
wherein a portion of the daughter expandable member carries the therapeutic agent and a portion of the daughter expandable member remains free of the therapeutic agent.

2. The method of claim 1, wherein the daughter expandable member remains free of a stent.

3. The method of claim 1, wherein the therapeutic agent is one or more of an anti-platelet agent, anti-inflammatory agent, anti-hyperlipidemic agent, anti-proliferative agent, an antibiotic, or anti-thrombogenic agent.

4. The method of claim 1, wherein the therapeutic agent comprises paclitaxel.

5. The method of claim 1, wherein the therapeutic agent is coupled with an excipient.

6. The method of claim 1, wherein the therapeutic agent is only carried on a distal portion of the daughter expandable member, and wherein a proximal portion of the daughter expandable member remains free of the therapeutic agent.

7. The method of claim 1, wherein a tapered region of the daughter expandable member carries the therapeutic agent.

8. The method of claim 1, wherein a tapered region of the daughter expandable member remains free of the therapeutic agent.

9. The method of claim 1, wherein the daughter expandable member is a balloon having a distal tapered region, a proximal tapered region, and a working length disposed therebetween, and wherein the therapeutic agent is disposed only on a distal portion of the working length, and the proximal tapered region and the distal tapered region remain free of the therapeutic agent.

10. The method of claim 1, wherein the mother expandable member or the daughter expandable member is a balloon.

11. The method of claim 1, wherein the daughter catheter further comprises a proximal marker, a distal marker, and an intermediate marker, each disposed on the daughter elongate shaft, the proximal, distal, and intermediate markers configured to be observed under an imaging modality,
wherein the proximal marker is axially aligned with a proximal portion of a working length of the daughter expandable member,
wherein the distal marker is axially aligned with a distal portion of a working length of the daughter expandable member, the distal portion of the working length carrying the therapeutic agent, and
wherein the intermediate marker is disposed between the proximal marker and the distal marker, and the intermediate marker is axially aligned with a proximal edge of the distal portion of the working length where the daughter expandable member stops carrying the therapeutic agent.

12. A system for treating a vessel with a bifurcation, the vessel having a mother vessel and a daughter vessel, the system comprising:
a mother catheter comprising a mother expandable member and a mother elongate shaft, the mother expandable member coupled to a distal portion of the mother elongate shaft;
a daughter catheter comprising a daughter expandable member and a daughter elongate shaft, the daughter expandable member coupled to a distal portion of the daughter elongate shaft; and
a therapeutic agent carried on the daughter expandable member,
wherein radial expansion of the daughter expandable member is configured to engage the daughter expandable member with a wall of the daughter vessel and the therapeutic agent elutes from the daughter expandable member into the wall.

13. The system of claim 12, wherein the therapeutic agent is one or more of an anti-platelet agent, anti-inflammatory agent, anti-hyperlipidemic agent, anti-proliferative agent, an antibiotic, or anti-thrombogenic agent.

14. The system of claim 12, wherein the therapeutic agent comprises paclitaxel.

15. The system of claim 12, wherein the therapeutic agent is coupled with an excipient.

16. The system of claim 12, wherein the therapeutic agent is only carried on a distal portion of the daughter expandable member, and wherein a proximal portion of the daughter expandable member remains free of the therapeutic agent.

17. The system of claim 12, wherein a tapered region of the daughter expandable member carries the therapeutic agent.

18. The system of claim 12, wherein a tapered region of the daughter expandable member remains free of the therapeutic agent.

19. The system of claim 12, wherein the daughter expandable member is a balloon having a distal tapered region, a proximal tapered region, and a working length disposed therebetween, and wherein the therapeutic agent is disposed only on a distal portion of the working length, and the proximal tapered region and the distal tapered region remain free of the therapeutic agent.

20. The system of claim 12, wherein the mother expandable member or the daughter expandable member is a balloon.

21. The system of claim 12, wherein the daughter catheter further comprises a proximal marker, a distal marker, and an intermediate marker, each disposed on the daughter elongate shaft,
wherein the proximal marker is axially aligned with a proximal portion of a working length of the daughter expandable member,
wherein the distal marker is axially aligned with a distal portion of a working length of the daughter expandable member, the distal portion of the working length carrying the therapeutic agent, and
wherein the intermediate marker is disposed between the proximal marker and the distal marker, and the intermediate marker is axially aligned with a proximal edge of the distal portion of the working length where the daughter expandable member stops carrying the therapeutic agent.

22. A method for treating a vessel with a bifurcation, the vessel having a main vessel and a branch vessel, the method comprising:
providing a delivery system having a first catheter with a first expandable member coupled to a distal portion of a first elongate shaft, and a second catheter with a second expandable member coupled to a distal portion of a second elongate shaft;
advancing the delivery system toward the bifurcation so that the first expandable member is at least partially disposed in the main vessel, and the second expandable member is at least partially disposed in the branch vessel;
radially expanding both the first expandable member and the second expandable member into engagement with walls of the main vessel and branch vessel;
eluting one or more therapeutic agents from at least one of the first expandable member, or the second expandable member.

23. The method of claim 22, wherein the one or more therapeutic agents are eluted from both the first expandable member and the second expandable member.

24. The method of claim 22, wherein the one or more therapeutic agents is eluted only from the first expandable member, and the second expandable member remains free of therapeutic agents.

25. The method of claim 22, wherein the one or more therapeutic agent is eluted only from the second expandable member, and the first expandable member remains free of therapeutic agents.

26. The method of claim 23, wherein the one or more therapeutic agents eluted from the first expandable member and the second expandable member are identical.

27. The method of claim 23, wherein the one or more therapeutic agents eluted from the first expandable member and the second expandable member are different.

28. The method of claim 27, wherein the first expandable member elutes an anti-proliferative agent, and the second expandable member elutes an anti-inflammatory agent.

29. The method of claim 27, wherein the first expandable member elutes a first therapeutic agent configured for sustained release and the second expandable member elutes a second therapeutic agent configured for burst release.

30. The method of claim 23, wherein a concentration of the therapeutic agent on the first expandable member is different from a concentration of the therapeutic agent on the second expandable member.

31. The method of claim 22, wherein the one or more therapeutic agents comprise a combination of two or more agents selected from anti-platelet agents, anti-inflammatory agents, anti-hyperlipidemic agents, anti-proliferative agents, antibiotics, and anti-thrombogenic agents.

32. A system for treating a vessel with a bifurcation, the vessel having a main vessel and a branch vessel, the system comprising:
a first catheter comprising a first expandable member and a first elongate shaft, the first expandable member coupled to a distal portion of the first elongate shaft;
a second catheter comprising a second expandable member and a second elongate shaft, the second expandable member coupled to a distal portion of the second elongate shaft; and
one or more therapeutic agents carried on at least one of the second expandable member or the first expandable member,
wherein radial expansion of the second expandable member or the first expandable member is configured to engage the second expandable member or the first expandable member with a wall of the branch vessel or a wall of the main vessel and the one or more therapeutic agents elutes from the second expandable member or the first expandable member into the corresponding wall of the branch vessel or the main vessel.

33. The system of claim 32, wherein the one or more therapeutic agents are eluted from both the first expandable member and the second expandable member.

34. The system of claim 32, wherein the one or more therapeutic agents is eluted only from the first expandable member, and the second expandable member remains free of therapeutic agents.

35. The system of claim 32, wherein the one or more therapeutic agent is eluted only from the second expandable member, and the first expandable member remains free of therapeutic agents.

36. The system of claim 33, wherein the one or more therapeutic agents eluted from the first expandable member and the second expandable member are identical.

37. The system of claim 33, wherein the one or more therapeutic agents eluted from the first expandable member and the second expandable member are different.

38. The system of claim 37, wherein the first expandable member elutes an anti-proliferative agent, and the second expandable member elutes an anti-inflammatory agent.

39. The system of claim 37, wherein the first expandable member elutes a first therapeutic agent configured for sustained release and the second expandable member elutes a second therapeutic agent configured for burst release.

40. The system of claim 33, wherein a concentration of the therapeutic agent on the first expandable member is different from a concentration of the therapeutic agent on the second expandable member.

41. The system of claim 32, wherein the one or more therapeutic agents comprise a combination of two or more agents selected from anti-platelet agents, anti-inflammatory agents, anti-hyperlipidemic agents, anti-proliferative agents, antibiotics, and anti-thrombogenic agents.

42. A method for treating a vessel with a bifurcation, the vessel having a main vessel and a branch vessel, the method comprising:
providing a delivery system having a first catheter with a first stent disposed over a first expandable member, and a second catheter with a second stent disposed over a second expandable member;
advancing the delivery system toward the bifurcation so that the first stent is positioned in the main vessel and the second stent is positioned in the branch vessel;
radially expanding both the first stent and the second stent into engagement with walls of the main vessel and branch vessel; and
eluting one or more therapeutic agents from at least one of the first stent, the first expandable member, the second stent, or the second expandable member.

43. The method of claim 42, wherein the one or more therapeutic agents are eluted from the first stent and the second stent.

44. The method of claim 42, wherein the one or more therapeutic agents are eluted from the first stent and the first expandable member.

45. The method of claim 42, wherein the one or more therapeutic agents are eluted from the first expandable member and the second expandable member.

46. The method of claim 42, wherein the one or more therapeutic agents are eluted from the second stent and the second expandable member.

47. A system for treating a vessel with a bifurcation, the vessel having a main vessel and a branch vessel, the system comprising:
a first catheter comprising a first expandable member, a first stent, and a first elongate shaft, the first stent disposed over a first expandable member;
a second catheter comprising a second expandable member, a second stent, and a second elongate shaft, the second stent disposed over a second expandable member; and
one or more therapeutic agents carried on at least one of the second expandable member, the second stent, the first stent, or the first expandable member,
wherein radial expansion of the second expandable member or the first expandable member is configured to engage the second stent or the first stent with a wall of the branch vessel or a wall of the main vessel and the one or more therapeutic agents elutes from the second expandable member, the second stent, the first stent, or the first expandable member into the corresponding wall of the branch vessel or the main vessel.

48. The system of claim 47, wherein the one or more therapeutic agents are eluted from the first stent and the second stent.

49. The system of claim 47, wherein the one or more therapeutic agents are eluted from the first stent and the first expandable member.

50. The system of claim 47, wherein the one or more therapeutic agents are eluted from the first expandable member and the second expandable member.

51. The system of claim 47, wherein the one or more therapeutic agents are eluted from the second stent and the second expandable member.
